SEQUENCE LISTING (50 sequences)

SEQ ID NO: 1 - RNA, 19 nt, Artificial
  siRNA targeting FOXA1
gagagaaaaaaucaacagc

SEQ ID NO: 2 - RNA, 19 nt, Artificial
  siRNA targeting FOXA1
gcacugcaauacucgccuu

SEQ ID NO: 3 - RNA, 19 nt, Artificial
  non-targeting control siRNA
uagcgacuaaacacaucaa

SEQ ID NO: 4 - DNA, 22 bp, Artificial
  oligonucleotide primer
ccgggaaactgtggcgtgatgg

SEQ ID NO: 5 - DNA, 25 bp, Artificial
  oligonucleotide primer
aggtggaggagtgggtgtcgctgtt

SEQ ID NO: 6 - DNA, 21 bp, Artificial
  oligonucleotide primer
agatggaagggcatgaaacca

SEQ ID NO: 7 - DNA, 20 bp, Artificial
  oligonucleotide primer
atgttgccgctcgtagtcat

SEQ ID NO: 8 - DNA, 20 bp, Artificial
  oligonucleotide primer
gttcatcctgggctccattg

SEQ ID NO: 9 - DNA, 19 bp, Artificial
  oligonucleotide primer
cccctggcatggttctctt

SEQ ID NO: 10 - DNA, 19 bp, Artificial
  oligonucleotide primer
gagaacgggaaggagacgc

SEQ ID NO: 11 - DNA, 19 bp, Artificial
  oligonucleotide primer
catctcagggtgaggggct

SEQ ID NO: 12 - DNA, 21 bp, Artificial
  oligonucleotide primer
gagaggacttgtctgcacatc

SEQ ID NO: 13 - DNA, 25 bp, Artificial
  oligonucleotide primer
gcatccacataaccatagataaagg

SEQ ID NO: 14 - DNA, 21 bp, Artificial
  oligonucleotide primer
agaaggtgggaaaatggtacc

SEQ ID NO: 15 - DNA, 20 bp, Artificial
  oligonucleotide primer
gttggcaaagcttcgaactg

SEQ ID NO: 16 - DNA, 23 bp, Artificial
  oligonucleotide primer
cagcttgactcaaaattcctgga

SEQ ID NO: 17 - DNA, 23 bp, Artificial
  oligonucleotide primer
tgaagattacgcttgcttttcct

SEQ ID NO: 18 - DNA, 20 bp, Artificial
  oligonucleotide primer
cacgcagaaaaccacacttc

SEQ ID NO: 19 - DNA, 21 bp, Artificial
  oligonucleotide primer
tgttcctccttgtgcatcttc

SEQ ID NO: 20 - DNA, 20 bp, Artificial
  oligonucleotide primer
tggtacatgtggctttgctc

SEQ ID NO: 21 - DNA, 21 bp, Artificial
  oligonucleotide primer
ccaccgagatgtcagaaagag

SEQ ID NO: 22 - DNA, 21 bp, Artificial
  oligonucleotide primer
tgcactgaggcagatgctgcg

SEQ ID NO: 23 - DNA, 22 bp, Artificial
  oligonucleotide primer
tcattgcggcacaccagtacag

SEQ ID NO: 24 - DNA, 24 bp, Artificial
  oligonucleotide primer
actgagtacaacaaagccatttga

SEQ ID NO: 25 - DNA, 23 bp, Artificial
  oligonucleotide primer
ttgtgacattgtcctgtccccac

SEQ ID NO: 26 - DNA, 21 bp, Artificial
  oligonucleotide primer
cctacattagtgccttacggg

SEQ ID NO: 27 - DNA, 22 bp, Artificial
  oligonucleotide primer
tccatttcgcagatagtacagc

SEQ ID NO: 28 - DNA, 19 bp, Artificial
  oligonucleotide primer
gagccgtacctgtcaccct

SEQ ID NO: 29 - DNA, 19 bp, Artificial
  oligonucleotide primer
gggccgtataggaacgtgc

SEQ ID NO: 30 - DNA, 20 bp, Artificial
  oligonucleotide primer
gcgcagatcacccagaagat

SEQ ID NO: 31 - DNA, 20 bp, Artificial
  oligonucleotide primer
tcctcaccaggatgctcaga

SEQ ID NO: 32 - DNA, 20 bp, Artificial
  oligonucleotide primer
agacaggaccatcggaatct

SEQ ID NO: 33 - DNA, 22 bp, Artificial
  oligonucleotide primer
gtaacccttcttcaggtggaac

SEQ ID NO: 34 - DNA, 88 bp, Artificial
  nucleic acid encoding a FOXA1 luciferase reporter
tcgatgtttacttacagtatgtttactttatccgtgtttacatagtctatatttacttaccatatgtttgcttagtcatgtttactca

SEQ ID NO: 35 - DNA, 60 bp, Artificial
  forkhead response element
acatattgtatcgattgtccttgacagtaaacaaatctgttgtaagagacattatcttta

SEQ ID NO: 36 - DNA, 32 bp, Artificial
  interferon-stimulated response element probe
ctcccctgagtttcacttcttctcccaacttg

SEQ ID NO: 37 - protein, 102 aa, Artificial
  fragment of a FOXA1 polypeptide
HAKPPYSYISLITMAIQQAPSKMLTLSEIYQWIMDLFPYYRQNQQRWQNSIRHSLSFNDCFVKVARSPDKPGKGSYWTLHPDSGNMFENGCYLRRQKRFKCE

SEQ ID NO: 38 - DNA, 17 bp, Artificial
  antisense oligonucleotide targeting FOXA1
  misc_feature (1)..(3): locked nucleic acids
  misc_feature (1)..(16): phosphorothioate (PS) backbone
  misc_feature (15)..(17): locked nucleic acids
atcagcatggccatcca

SEQ ID NO: 39 - DNA, 19 bp, Artificial
  antisense oligonucleotide targeting FOXA1
  misc_feature (1)..(3): locked nucleic acids
  misc_feature (1)..(18): PS backbone
  misc_feature (17)..(19): locked nucleic acids
accacccgttctccatcaa

SEQ ID NO: 40 - DNA, 20 bp, Artificial
  antisense oligonucleotide targeting FOXA1
  misc_feature (1)..(3): locked nucleic acids
  misc_feature (1)..(19): PS backbone
  misc_feature (18)..(20): locked nucleic acids
actcgccttacggctctacg

SEQ ID NO: 41 - DNA, 23 bp, Artificial
  antisense oligonucleotide targeting FOXA1
  misc_feature (1)..(3): locked nucleic acids
  misc_feature (1)..(22): PS backbone
  misc_feature (21)..(23): locked nucleic acids
ccattttaatcattgccatcgtg

SEQ ID NO: 42 - DNA, 20 bp, Artificial
  antisense oligonucleotide targeting FOXA1
  misc_feature (1)..(3): locked nucleic acids
  misc_feature (1)..(19): PS backbone
  misc_feature (18)..(20): locked nucleic acids
ggtagcgccataaggagagt

SEQ ID NO: 43 - DNA, 16 bp, Artificial
  antisense oligonucleotide targeting FOXA1
  misc_feature (1)..(3): locked nucleic acids
  misc_feature (1)..(15): PS backbone
  misc_feature (14)..(16): locked nucleic acids
tggatggccatcgtga

SEQ ID NO: 44 - DNA, 17 bp, Artificial
  control antisense oligonucleotide
  misc_feature (1)..(3): locked nucleic acids
  misc_feature (1)..(16): PS backbone
  misc_feature (15)..(17): locked nucleic acids
gacgcgcctgagaggtt

SEQ ID NO: 45 - DNA, 17 bp, Artificial
  antisense oligonucleotide targeting FOXA1
atcagcatggccatcca

SEQ ID NO: 46 - DNA, 19 bp, Artificial
  antisense oligonucleotide targeting FOXA1
accacccgttctccatcaa

SEQ ID NO: 47 - DNA, 20 bp, Artificial
  antisense oligonucleotide targeting FOXA1
actcgccttacggctctacg

SEQ ID NO: 48 - DNA, 23 bp, Artificial
  antisense oligonucleotide targeting FOXA1
ccattttaatcattgccatcgtg

SEQ ID NO: 49 - DNA, 20 bp, Artificial
  antisense oligonucleotide targeting FOXA1
ggtagcgccataaggagagt

SEQ ID NO: 50 - DNA, 16 bp, Artificial
  antisense oligonucleotide targeting FOXA1
tggatggccatcgtga

(19) United States
(12) Patent Application Publication
(10) Pub. No.: US 2023/0079757 A1
(43) Pub. Date: Mar. 16, 2023

(54) METHODS AND MATERIALS FOR TREATING CANCER

(71) Applicant: Mayo Foundation for Medical Education and Research, Rochester, MN (US)

(72) Inventors: Haojie Huang, Rochester, MN (US); Yundong He, Rochester, MN (US); Matthew P. Goetz, Rochester, MN (US)

(21) Appl. No.: 17/800,008

(22) PCT Filed: Feb. 18, 2021
(86) PCT No.: PCT/US2021/018484

§ 371 Date: Aug. 16, 2022

Related U.S. Application Data
(60) Provisional application No. 62/978,208, filed on Feb. 18, 2020.

(51) Int. Cl. C12N 15/113; A61K 39/395; A61K 31/4245; A61K 38/14; A61K 31/69; A61K 31/282; A61K 31/7068; A61K 33/243; A61K 31/136; A61K 31/704; A61K 31/513; A61K 31/506; A61K 31/337; A61K 31/454; A61K 31/404; A61K 31/517; A61K 31/519; A61K 31/4709; A61K 31/5377; A61K 31/4745; A61K 31/453; A61K 38/12; A61P 35/00; G01N 33/574

(52) U.S. Cl. CPC C12N 15/113; A61K 39/3955; A61K 31/4245; A61K 38/14; A61K 31/69; A61K 31/282; A61K 31/7068; A61K 33/243; A61K 31/136; A61K 31/704; A61K 31/513; A61K 31/506; A61K 31/337; A61K 31/454; A61K 31/404; A61K 31/517; A61K 31/519; A61K 31/4709; A61K 31/5377; A61K 31/4745; A61K 31/453; A61K 38/12; A61P 35/00; G01N 33/57484; C12N 2310/14; C12N 2310/11; G01N 2333/4706

(57) ABSTRACT

This document relates to methods and materials involved in assessing and/or treating mammals (e g , humans) having cancer. For example, methods and materials that can be used to determine whether or not the cancer is likely to be responsive to a particular cancer treatment (e.g., a cancer immunotherapy or a cancer chemotherapy) are provided. For example, methods and materials that can be used to treat a mammal by administering one or more cancer treatments that is/are selected based, at least in part, on whether or not the mammal is likely to be responsive to a particular cancer treatment also are provided.

### CROSS-REFERENCE To RELATED APPLICATIONS

This application claims the benefit of U.S. Patent Application Ser. No. 62/978,208, filed on Feb. 18, 2020. The disclosure of the prior application is considered part of (and is incorporated by reference in) the disclosure of this application.

### BACKGROUND

### 1. Technical Field

This document relates to methods and materials involved in assessing and/or treating mammals (e.g., humans) having cancer. For example, methods and materials provided herein can be used to determine whether or not a cancer is likely to be responsive to a particular cancer treatment (e.g., a cancer immunotherapy or a cancer chemotherapy). In some cases, the methods and materials provided herein can be used to treat a mammal by administering, to the mammal, one or more cancer treatments that is/are selected based, at least in part, on whether or not the mammal is likely to be responsive to a particular cancer treatment.

### 2. Background Information

Infiltration of lymphocytes in tumors is an essential step in immune attack of cancer cells. Indeed, the abundance of tumor-infiltrating lymphocytes (TILs) is a valuable prognostic factor for both chemotherapy and immune checkpoint inhibitor (ICI) therapy (Adams et al., J Clin Oncol 32:2959-2966 (2014); Loi et al., Ann Oncol 25:1544-1550 (2014); and Denkert et al., J Clin Oncol 28:105-113 (2010)). Cytotoxic lymphocytes (CTL), mainly cytotoxic T (Tc) and natural killer (NK) cells utilize granule exocytosis as a common mechanism to destroy cancer cells by expressing and releasing the pore forming proteins including perforin 1 (PRF1), granule-associated enzymes (granzymes (GZMs)) and natural killer cell granule protein 7 (NKG7) (Martinez-Lostao, Clinical Cancer Research 21:5047-5056 (2015)). Prostate and breast cancer are generally immunologically “cold.”

### SUMMARY

This document provides methods and materials involved in assessing and/or treating mammals (e.g., humans) having cancer. In some cases, this document provides methods and materials for determining whether or not a mammal having cancer is likely to be responsive to a particular cancer treatment (e.g., one or more cancer immunotherapies and/or one or more cancer chemotherapies), and, optionally, administering one or more cancer therapies that is/are selected based, at least in part, on whether or not the mammal is likely to be responsive to a particular cancer treatment to the mammal. For example, a sample (e.g., a sample containing one or more cancer cells) obtained from a mammal (e.g., a human) having cancer can be assessed to determine if the mammal is likely to be responsive to a particular cancer treatment based, at least in part, on the presence, absence, or level of Forkhead box protein A1 (FOXA1) polypeptide expression in the sample.

As demonstrated herein, overexpression of a FOXA1 coding sequence (e.g., resulting in an increased level of FOXA1 polypeptides) can be used to identify cancer patients (e.g., breast cancer patients such as triple negative breast cancer (TNBC) patients, prostate cancer patients, and/or bladder cancer patients) as having immunotherapy resistance and/or chemo-resistance. These results demonstrate that increased expression of a FOXA1 polypeptide can used to determine immunotherapy (e.g., immune checkpoint inhibitor (ICI)-based immunotherapy) responsiveness. These results also demonstrate that a FOXA1 polypeptide (and/or nucleic acid encoding a FOXA1 polypeptide) can be used as a therapeutic target to overcome immunotherapy resistance and/or chemotherapy resistance in a cancer.

Having the ability to determine whether or not a particular patient is likely to respond to a particular cancer treatment (e.g., a cancer immunotherapy or a cancer chemotherapy) allows clinicians to provide an individualized approach in selecting cancer treatments for that patient. Further, having the ability to convert “cold” tumors (e.g., tumors that are not recognized by the immune system) into “hot” tumors (e.g., tumors that can be recognized by the immune system) as described herein (e.g., by administering one or more inhibitors of a FOXA1 polypeptide) can allow clinicians and patients use new and unique ways to treat cancers that are otherwise resistant to immunotherapies and/or chemotherapies.

In general, one aspect of this document features a method for assessing a mammal having cancer. The method comprises, consists essentially of, or consists of (a) detecting a presence or absence of an increased level of Forkhead box protein A1 (FOXA1) polypeptide expression in a sample from the mammal; (b) classifying the mammal as not being likely to respond to an immunotherapy or a chemotherapy if the presence of the increased level is detected, and (c) classifying the mammal as being likely to respond to the immunotherapy or the chemotherapy if the absence of the increased level is detected. The mammal can be a human. The sample can comprise cancer cells of the cancer. The cancer can be selected from the group consisting of a prostate cancer, a breast cancer, a bladder cancer, a lung cancer, a liver cancer, a cervical cancer, a bile duct cancer, a colon cancer, a rectal cancer, a pancreatic cancer, a uterine cancer, a head and neck cancer, a testicular cancer, a ovarian cancer, a thyroid cancer, a bone cancer, a skin cancer, an adrenal gland cancer, a kidney cancer, a lymphoma, a thymus cancer, a brain cancer, a leukemia, and a cancer of the eye. The method can comprise detecting the presence of the increased level. The method can comprise classifying the mammal as not being likely to respond to the immunotherapy or the chemotherapy. The method can comprise detecting the absence of the increased level. The method can comprise classifying the mammal as being likely to respond to the immunotherapy or the chemotherapy. The detecting step can comprise performing a method that detects FOXA1 polypeptides in the sample using an anti-FOXA1 polypeptide antibody. The detecting step can comprise performing a method that detects mRNA encoding an FOXA1 polypeptide.

In another aspect, this document features a method for treating a mammal having cancer. The method comprises, consists essentially of, or consists of (a) detecting an increased level of FOXA1 polypeptide expression in a sample obtained from the mammal; and (b) administering a cancer treatment to the mammal, wherein the cancer treatment is not an immunotherapy or a chemotherapy. The mammal can be a human. The sample can comprise cancer cells of the cancer. The cancer can be selected from the group consisting of a prostate cancer, a breast cancer, a bladder cancer, a lung cancer, a liver cancer, a cervical cancer, a bile duct cancer, a colon cancer, a rectal cancer, a pancreatic cancer, a uterine cancer, a head and neck cancer, a testicular cancer, a ovarian cancer, a thyroid cancer, a bone cancer, a skin cancer, an adrenal gland cancer, a kidney cancer, a lymphoma, a thymus cancer, a brain cancer, a leukemia, and a cancer of the eye. The cancer treatment can comprise surgery. The cancer treatment can comprise radiation treatment.

In another aspect, this document features a method for treating cancer. The method comprises, consists essentially of, or consists of administering a cancer treatment to a mammal identified as having an increased level of FOXA1 polypeptide expression in a sample obtained from the mammal, wherein the cancer treatment is not an immunotherapy or a chemotherapy. The mammal can be a human. The sample can comprise cancer cells of the cancer. The cancer can be selected from the group consisting of a prostate cancer, a breast cancer, a bladder cancer, a lung cancer, a liver cancer, a cervical cancer, a bile duct cancer, a colon cancer, a rectal cancer, a pancreatic cancer, a uterine cancer, a head and neck cancer, a testicular cancer, a ovarian cancer, a thyroid cancer, a bone cancer, a skin cancer, an adrenal gland cancer, a kidney cancer, a lymphoma, a thymus cancer, a brain cancer, a leukemia, and a cancer of the eye. The cancer treatment can comprise surgery. The cancer treatment can comprise radiation treatment.

In another aspect, this document features a method for treating a mammal having cancer. The method comprises, consists essentially of, or consists of (a) detecting an absence of an increased level of FOXA1 polypeptide expression in a sample obtained from the mammal; and (b) administering a cancer treatment to the mammal, wherein the cancer treatment is an immunotherapy or a chemotherapy. The mammal can be a human. The sample can comprise cancer cells of the cancer. The cancer can be selected from the group consisting of a prostate cancer, a breast cancer, a bladder cancer, a lung cancer, a liver cancer, a cervical cancer, a bile duct cancer, a colon cancer, a rectal cancer, a pancreatic cancer, a uterine cancer, a head and neck cancer, a testicular cancer, a ovarian cancer, a thyroid cancer, a bone cancer, a skin cancer, an adrenal gland cancer, a kidney cancer, a lymphoma, a thymus cancer, a brain cancer, a leukemia, and a cancer of the eye. The cancer treatment can comprise an immunotherapy selected from the group consisting of pembrolizumab, nivolumab, cemiplimab, spartalizumab, camrelizumab, sintilimab, tislelizumab, toripalimab, AMP-224, AMP-514, atezolizumab, avelumab, durvalumab, KN035, CK-301, AUNP12, CA-170, and BMS-986189. The cancer treatment can comprise a chemotherapy selected from the group consisting of actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, and vindesine.

In another aspect, this document features a method for treating cancer. The method comprises, consists essentially of, or consists of administering a cancer treatment to a mammal identified as lacking an increased level of FOXA1 polypeptide expression in a sample obtained from the mammal, wherein the cancer treatment is an immunotherapy or a chemotherapy. The mammal can be a human. The sample can comprise cancer cells of the cancer. The cancer can be selected from the group consisting of a prostate cancer, a breast cancer, a bladder cancer, a lung cancer, a liver cancer, a cervical cancer, a bile duct cancer, a colon cancer, a rectal cancer, a pancreatic cancer, a uterine cancer, a head and neck cancer, a testicular cancer, a ovarian cancer, a thyroid cancer, a bone cancer, a skin cancer, an adrenal gland cancer, a kidney cancer, a lymphoma, a thymus cancer, a brain cancer, a leukemia, and a cancer of the eye. The cancer treatment can comprise an immunotherapy selected from the group consisting of pembrolizumab, nivolumab, cemiplimab, spartalizumab, camrelizumab, sintilimab, tislelizumab, toripalimab, AMP-224, AMP-514, atezolizumab, avelumab, durvalumab, KN035, CK-301, AUNP12, CA-170, and BMS-986189. The cancer treatment can comprise a chemotherapy selected from the group consisting of actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, and vindesine.

In another aspect, this document features a method for treating a mammal having cancer. The method comprises, consists essentially of, or consists of (a) detecting an increased level of FOXA1 polypeptide expression in a sample obtained from the mammal; (b) administering an inhibitor of a FOXA1 polypeptide; and (c) administering a cancer treatment to the mammal, wherein the cancer treatment is an immunotherapy or a chemotherapy. The mammal can be a human. The sample can comprise cancer cells of the cancer. The cancer can be selected from the group consisting of a prostate cancer, a breast cancer, a bladder cancer, a lung cancer, a liver cancer, a cervical cancer, a bile duct cancer, a colon cancer, a rectal cancer, a pancreatic cancer, a uterine cancer, a head and neck cancer, a testicular cancer, a ovarian cancer, a thyroid cancer, a bone cancer, a skin cancer, an adrenal gland cancer, a kidney cancer, a lymphoma, a thymus cancer, a brain cancer, a leukemia, and a cancer of the eye. The inhibitor of the FOXA1 polypeptide can be an inhibitor of FOXA1 polypeptide activity. The inhibitor of the FOXA1 polypeptide activity can be SNS-032 (BMS-387032), Ro 31-8220, Aurora A Inhibitor I, WZ8040, Dasatinib, Lapatinib, Saracatinib (AZD0530), JNK-IN-8, BI 2536, Crenolanib (CP-868596), Herceptin, CYT387, BEZ235 (Dactolisib), PHA-793887, NVP-BSK805 2HCl, Cediranib (AZD2171), PF-00562271, Flavopiridol, AT7519, Apicidin, or Volasertib (BI 6727). The inhibitor of the FOXA1 polypeptide can be an inhibitor of FOXA1 polypeptide expression. The inhibitor of the FOXA1 polypeptide expression can be a small interfering RNA (siRNA) molecule or an antisense oligo. The siRNA can comprise or consist of nucleic acid selected from the group consisting of GAGAGAAAAAAUCAACAGC (SEQ ID NO:1) and GCACUGCAAUACUCGCCUU (SEQ ID NO:2). Administering the inhibitor of the FOXA1 polypeptide can comprise administering a viral particle comprising the shRNA to the mammal. The antisense oligo can comprise or consist of nucleic acid selected from the group consisting of SEQ ID NO:38, SEQ ID NO:39, SEQ ID NO:40, SEQ ID NO:41, SEQ ID NO:42, SEQ ID NO:42, SEQ ID NO:43, ATCAGCATGGCCATCCA (SEQ ID NO:45), ACCACCCGTTCTCCATCAA (SEQ ID NO:46), ACTCGCCTTACGGCTCTACG (SEQ ID NO:47), CCATTTTAATCATTGCCATCGTG (SEQ ID NO:48), GGTAGCGCCATAAGGAGAGT (SEQ ID NO:49), and TGGATGGCCATCGTGA (SEQ ID NO:50). The cancer treatment can comprise an immunotherapy selected from the group consisting of pembrolizumab, nivolumab, cemiplimab, spartalizumab, camrelizumab, sintilimab, tislelizumab, toripalimab, AMP-224, AMP-514, atezolizumab, avelumab, durvalumab, KN035, CK-301, AUNP12, CA-170, and BMS-986189. The cancer treatment can comprise a chemotherapy selected from the group consisting of actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, and vindesine.

In another aspect, this document features a method for treating cancer. The method comprises, consists essentially of, or consists of administering an inhibitor of a FOXA1 polypeptide to a mammal identified as having an increased level of FOXA1 polypeptide expression in a sample obtained from the mammal, and administering a cancer treatment to the mammal, wherein the cancer treatment is an immunotherapy or a chemotherapy. The mammal can be a human. The sample can comprise cancer cells of the cancer. The cancer can be selected from the group consisting of a prostate cancer, a breast cancer, a bladder cancer, a lung cancer, a liver cancer, a cervical cancer, a bile duct cancer, a colon cancer, a rectal cancer, a pancreatic cancer, a uterine cancer, a head and neck cancer, a testicular cancer, a ovarian cancer, a thyroid cancer, a bone cancer, a skin cancer, an adrenal gland cancer, a kidney cancer, a lymphoma, a thymus cancer, a brain cancer, a leukemia, and a cancer of the eye. The inhibitor of the FOXA1 polypeptide can be an inhibitor of FOXA1 polypeptide activity. The inhibitor of the FOXA1 polypeptide activity can be SNS-032 (BMS-387032), Ro 31-8220, Aurora A Inhibitor I, WZ8040, Dasatinib, Lapatinib, Saracatinib (AZD0530), JNK-IN-8, BI 2536, Crenolanib (CP-868596), Herceptin, CYT387, BEZ235 (Dactolisib), PHA-793887, NVP-BSK805 2HCl, Cediranib (AZD2171), PF-00562271, Flavopiridol, AT7519, Apicidin, or Volasertib (BI 6727). The inhibitor of the FOXA1 polypeptide can be an inhibitor of FOXA1 polypeptide expression. The inhibitor of the FOXA1 polypeptide expression can be a siRNA molecule or an antisense oligo. The siRNA can comprise or consist of nucleic acid selected from the group consisting of GAGAGAAAAAAUCAACAGC (SEQ ID NO:1) and GCACUGCAAUACUCGCCUU (SEQ ID NO:2). Administering the inhibitor of the FOXA1 polypeptide can comprise administering a viral particle comprising the shRNA to the mammal. The antisense oligo can comprise or consist of nucleic acid selected from the group consisting of SEQ ID NO:38, SEQ ID NO:39, SEQ ID NO:40, SEQ ID NO:41, SEQ ID NO:42, SEQ ID NO:42, SEQ ID NO:43, ATCAGCATGGCCATCCA (SEQ ID NO:45), ACCACCCGTTCTCCATCAA (SEQ ID NO:46), ACTCGCCTTACGGCTCTACG (SEQ ID NO:47), CCATTTTAATCATTGCCATCGTG (SEQ ID NO:48), GGTAGCGCCATAAGGAGAGT (SEQ ID NO:49), and TGGATGGCCATCGTGA (SEQ ID NO:50). The cancer treatment can comprise an immunotherapy selected from the group consisting of pembrolizumab, nivolumab, cemiplimab, spartalizumab, camrelizumab, sintilimab, tislelizumab, toripalimab, AMP-224, AMP-514, atezolizumab, avelumab, durvalumab, KN035, CK-301, AUNP12, CA-170, and BMS-986189. The cancer treatment can comprise a chemotherapy selected from the group consisting of actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, and vindesine.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1C. FOXA1 levels inversely correlate with immune response gene expression in cancer. FIG. 1A) List of top 10 genes whose expression negatively correlated to the level of T cell effector genes PRF 1, GZMA and NKG7 in prostate and breast cancer of TCGA cohorts. FIG. 1B) Genes and pathways negatively (Spearman's rho <−0.4) correlated with FOXA1 expression in prostate and breast cancers of TCGA cohorts revealed by Gene Ontology Biological Processes (GO-BP) analysis. FIG. 1C) Heatmaps show the inverse correlation between FOXA1 expression and the levels of CD8⁺ T effector cell (CD8⁺ T_{eff}) signature genes and antigen processing and presentation machinery (APM) genes in prostate cancer of TCGA, SU2C, and PROMOTE cohorts. Samples are ranked based on FOXA1 transcript levels.

FIGS. 2A-2E. FOXA1 gene expression negatively correlates with the level of immune response genes in prostate and breast cancer patients. FIG. 2A) List of top 10 genes whose expression negatively correlated to the level of T cell effector genes GZMB, GZMH, and GZMM in prostate and breast cancer of TCGA cohorts. FIG. 2B and FIG. 2C) Spearman's rho analysis shows the inverse correlation between FOXA1 level and expression of CD8 effector cell (CD8⁺ T_{eff}) signature genes (FIG. 2B) and antigen presentation machinery (APM) genes (FIG. 2C) in prostate and breast cancer from the TCGA cohorts. FIG. 2D) The correlation revealed by Spearman's rho analysis between FOXA1 level and expression of each of CD8 effector cell (CD8⁺ T_{eff}) signature genes examined in prostate and breast cancer from the TCGA cohorts. FIG. 2E) The correlation revealed by Spearman's rho analysis between FOXA1 level and expression of each of APM genes examined in prostate and breast cancer from the TCGA cohorts.

FIGS. 3A-3G. FOXA1 negatively correlates with immune response genes in prostate and breast cancer patients. FIGS. 3A-3C) The correlation between FOXA1 level and the expression of CD8 effector cell (CD8⁺ T_{eff} score (combined all the CD8⁺ T_{eff} signature genes listed in FIG. 1C) and antigen presentation machinery (APM) score (combined all the APM genes listed in FIG. 1C) in prostate cancer from TCGA database, SU2C database, and PROMOTE database. FIGS. 3D-3G) The correlation between FOXA1 expression and the level of CD8 effector cell (CD8⁺ T_{eff}) signature genes and antigen presentation machinery (APM) genes in breast cancer from TCGA database and METABRIC database.

FIGS. 4A-4D. FOXA1 is overexpressed in prostate and breast cancer in patients. FIG. 4A) Comparison of FOXA1 mRNA level among 31 types of cancer from TCGA cohorts, including PRAD (prostate adenocarcinoma), BRCA (breast invasive carcinoma), BLCA (bladder urothelial carcinoma), LUAD (lung adenocarcinoma), LIHC (liver hepatocellular carcinoma), CESC (cervical squamous cell carcinoma and endocervical adenocarcinoma), CHOL (cholangiocarcinoma), LUSC (lung squamous cell carcinoma), COAD (colon adenocarcinoma), READ (rectum adenocarcinoma), PAAD (pancreatic adenocarcinoma), UCEC (uterine corpus endometrial carcinoma), UCS (uterine carcinosarcoma), HNSC (head and neck squamous cell carcinoma), MESO (mesothelioma), TGCT (testicular germ cell tumors), OV (ovarian serous cystadenocarcinoma), THCA (thyroid carcinoma), SARC (sarcoma), SKCM (skin cutaneous melanoma), ACC (adrenocortical carcinoma), KIRC (kidney renal clear cell carcinoma), PCPG (pheochromocytoma and paraganglioma), KIRP (kidney renal papillary cell carcinoma), DLBC (lymphoid neoplasm diffuse large B-cell lymphoma), THYM (thymoma), LGG (brain lower grade glioma), KICH (kidney chromophobe), GBM (glioblastoma multiforme), LAME (acute myeloid leukemia), UVM (uveal melanoma). FIGS. 4B-4C) Comparison of FOXA1 mRNA level between normal tissues and prostate (FIG. 4B) and breast cancer (FIG. 4C) of the indicated cohorts. FIG. 4D) Comparison of CD274 (PD-L1) mRNA level between normal tissues and prostate cancer and breast cancer of the TCGA cohorts.

FIGS. 5A-5D. FOXA1 negatively regulates interferon signaling pathway. FIG. 5A) FOXA1 inhibits the activity of interferon (type I and III)-stimulated response element luciferase reporter (ISRE-luc) and IFN-y (type 11)-activated sequences luciferase reporter (GAS-luc) in 293T cells. Data shown as means ±s.d. (n=3). FIG. 5B) Western blot analysis of FOXA1 and AR in the indicated cell lines. ERK2 was used as a loading control. FIG. 5C) Heatmaps show the inverse correlation of FOXA1 expression with levels of Type I IFN response signature genes in prostate cancer of TCGA, SU2C, and PROMOTE cohorts. FIG. 5D) RT-qPCR analysis of indicated IFN signaling genes in the indicated cells treated with or without IFNα. Data shown as means ±s.d. (n=3). Statistical significance was determined by unpaired two-tailed Student's t tests.

FIGS. 6A-6E. FOXA1 negatively regulates interferon signaling pathway. FIG. 6A) The correlation between FOXA1 mRNA level and the Type I IFN response activity (combined all the Type I IFN response signature genes listed in FIG. 5C) in prostate cancer of the TCGA, SU2C, and PROMOTE cohorts. FIGS. 6B-6E) The correlation between FOXA1 mRNA level and the expression level of Type I IFN response signature genes in breast cancer from the TCGA and METABRIC cohorts.

FIGS. 7A-7B. FOXA1 negatively correlates with immune response gene expression in bladder cancer patients. FIG. 7A) Spearman's rho test shows the inverse correlation between FOXA1 expression level and CD8 effector cell (CD8⁺ T_{eff}) signature gene expression and antigen presentation machinery (APM) genes expression in bladder cancers of the TCGA cohort. FIG. 7B) Heatmaps shows the correlation between FOXA1 level and the expression level of CD8 effector cell (CD8⁺ T_{eff}) signature genes, antigen presentation machinery (APM) genes and Type I IFN response signature genes in bladder cancers of the TCGA cohort.

FIGS. 8A-8G. FOXA1 impedes IFNa-induced STAT2 binding to its target gene loci. FIG. 8A) Co-IP shows the interaction of endogenous FOXA1 with endogenous STAT 1 and STAT2 in LNCaP cells treated with IFNα or IFNγ. FIG. 8B) Diagram shows expression constructs for FOXA1 truncation and missense mutants within a fragment of a FOXA1 polypeptide (SEQ ID NO:37). NLS, nuclear localization signal.

FIGS. 8C and 8D) GST pulldown assay shows the interaction of STAT2 DNA binding domain (STAT2-DBD) with the indicated FOXA1 mutants. FIG. 8E) Inhibitory effect of the indicated FOXA1 WT or mutants on ISRE-luc reporter gene activity. Data shown as means ±s.d. (n=3). Statistical significance was determined by unpaired two-tailed Student's t tests. FIG. 8F) Heatmaps show STAT2 ChIP-seq signaling in LNCaP cells under different treatment conditions. FIG. 8G) Western blot analysis of indicated proteins in LNCaP cells under different treatment conditions. ERK2 was used as a loading control.

FIGS. 9A-9J. FOXA1 inhibits IFNa-induced DNA binding ability of STAT2. FIGS. 9A and 9B) Co-IP shows the interaction of endogenous FOXA1 with endogenous STAT1 and STAT2 in MCF7 (breast cancer) (FIG. 9A) and RT4 (bladder cancer) cells (FIG. 9B) treated with IFNα or IFNγ. FIG. 9C) Co-IP analysis of ectopically expressed proteins shows the interaction of FOXA1 with STAT1 and STAT2. FIG. 9D) Co-IP analysis shows the effect of FOXA1 on the formation of STAT1-STAT2-IRF9 and STAT1-STAT1 complexes. FIG. 9E) GST pulldown assays show the interaction of the DNA binding domain (DBD) of STAT2 with the Forkhead domain-containing region (FOXA1-FKCR). FIG. 9F) Co-IP analysis of interaction of FOXA1 truncation mutants FOXA1(141-294) and FOXA1(141-247) with STAT1 and STAT2 in 293T cells treated with IFNα. FIG. 9G) Effect of FOXA1 truncation mutants on interferon-stimulated response element luciferase reporter (ISRE-luc) activity in 293T cells treated with IFNα. FIG. 9H) Effect of the indicated FOXA1 mutants on FOXA1 response element luciferase reporter (KLK3 enhancer reporter) activity in 293T cells. FIG. 91) Analysis of binding of FOXA1 WT and indicated mutants to the forkhead response element in the KLK3 enhancer using electrophoretic mobility shift assay (EMSA). FIG. 9J) Western blot analysis of indicated proteins in LNCaP cells transfected with control (siCon) or FOXA1-specific siRNA (siFOXA1) in combination with restored expression of FOXA1 WT or indicated mutants. Independent sets of cells were also used for STAT2 ChIP-seq as shown in FIG. 8F.

FIGS. 10A-10C. FOXA1 impairs IFNa-induced DNA binding ability of STAT2. FIG. 10A) EMSA assessment of the effect of FOXA1 WT and DNA binding-deficient mutant FOXA1ΔαH3 on the formation of DNA (interferon-stimulated response element, ISRE)-protein complexes. FIG. 10B) UCSC tracks profiles of STAT2 ChIP-seq signals (signal per ten million reads, SPTMR) at the indicated gene loci (ISG15, MX1, IRF9, IFI44L and IFITM1) in LNCaP cells transfected/infected with siRNAs or expression vectors as indicated. FIG. 10C) ChIP-seq read intensity heatmaps show genome-wide FOXA1 chromatin binding signals in LNCaP cells treated with or without IFNα.

FIGS. 11A-11F. Effects of prostate cancer-derived FOXA1 mutants on expression of IFN signature genes, APM genes and CD8⁺ T effector genes. FIG. 11A) Western blot analysis the effect of expression of indicated siRNA and expression vectors on IFNα-induced expression of IFN response genes in VCaP cells. ERK2 was used as a loading control. FIGS. 11B and 11C) Western blot analysis of the effect of FOXA1 knockdown on IFNα-induced expression of IFN response genes in MCF7 (breast cancer) (FIG. 11B) and RT4 (bladder cancer) cells (FIG. 11C). ERK2 was used as a loading control. FIG. 11D) Effect of FOXA1-WT and prostate cancer-derived mutant FOXA1-H247Q, FOXA1-R261G and FOXA1-F266L on interferon-stimulated response element luciferase reporter (ISRE-luc) activity (for type I and III IFN response) and IFN-γ-activated sequences luciferase reporter (GAS-luc) activity (for type II IFN response) in 293T cells treated with IFNα or IFNγ. FIG. 11E) Co-IP analysis of interaction of ectopically expressed FOXA1-WT and FOXA1-H247Q, FOXA1-R261G and FOXA1-F266L mutants with STAT2 in 293T cells. FIG. 11F) Comparison of CD8⁺ T effector signature gene expression score (CD8⁺ T_{eff} score), antigen presentation machinery gene expression score (APM score) and Type I IFN response gene expression score/activity in between FOXA1 WT and mutated samples of prostate, breast or bladder cancer in the TCGA cohorts.

FIGS. 12A-12C. Effects of FOXA1 WT, prostate cancer-derived mutant and DNA binding-deficient mutant on expression of IFN signature genes in TRAMP-C2 murine prostate cancer cells in culture and T cell infiltration in TRAMP-C2 tumors in mice. FIG. 12A) Western blot analysis of indicated proteins in TRAMP-C2 cells transfected with indicated expression vectors and treated with or without IFNα. Erk2 was used as a loading control. FIG. 12B) Flow cytometry analysis of expression of APM protein MHC class I (H-2Kd/H-2Dd) on the surface of vehicle or IFNα-treated TRAMP-C2 cells expressing the indicated expression vectors. FIG. 12C) Immunofluorescence chemistry-based examination of expression of the transfected FOXA1ΔαH3 expression in TRAMP-C2-Vector and TRAMP-C2-FOXA1ΔαH3 tumors from mice at 2 day after the last vehicle or Poly(I:C) administration.

FIGS. 13A-13H. FOXA1 overexpression confers cancer immuno- and chemo-therapy resistance in mice and patients. FIG. 13A) Schematic diagram of generation and Poly(I:C) treatment of TRAMP-C2 prostate tumors in syngeneic mice. FIG. 13B) Growth of TRAMP-C2-Vector and TRAMP-C2-FOXA1ΔαH3 tumors treated with or without Poly(I:C). FIG. 13C) Tumor-free survival of syngeneic mice bearing TRAMP-C2-Vector or TRAMP-C2-FOXA1ΔαH3 tumors administrated with or without Poly(I:C). Statistical significance was determined by Log-rank (Mantel-Cox) test. FIGS. 13D and 13E) PhenoGraph-defined cellular distribution and clustering, as defined by tSNE1 and tSNE2, colored by cellular phenotypes in TRAMP-C2-Vector and TRAMP-C2-FOXA1ΔαH3 tumors from mice at 16 days post vehicle or Poly(I:C) treatment (FIG. 13D). Data were derived from all normalized viable single cells, subjected to the PhenoGraph algorithm. Data in the bar graphs (FIG. 13E) are means ±s.d, n=3. FIG. 13F) RNA-seq data (GSE124821) analysis shows the correlation of expression of Foxa1, CD3e, CD8a and Gzmb in a cohort of 204 murine triple-negative breast cancers with the responsiveness to anti-PD1 and anti-CTLA-4 combination. FIG. 13G) RNA-seq data analysis shows the association of expression of FOXA1, CD3E, CD8A and GZMB in a cohort of 126 breast cancers of patients who underwent neoadjuvant chemotherapy (NAC) with pathological complete response (pCR as indicated by residual cancer burden (RCB, grade 0)) versus no pCR (RCB, grade I, II or III). Statistical significance was determined by unpaired two-tailed Student's t tests in FIGS. 13E, 13F and 13G. FIG. 13H) Progression-free survival of patients with FOXA1-expression low or high urothelial carcinoma treated with anti-PD1 immunotherapy. Statistical significance was determined by Log-rank (Mantel-Cox) test.

FIGS. 14A-14D. FOXA1 expression and overall gene mutation burden in breast cancer in patients. FIG. 14A) Comparison of FOXA1 mRNA level between triple-negative breast cancer (TNBC) and other types of breast cancer from the METABRIC cohort. FIG. 14B) Comparison of DNA mutational load in breast cancers from a cohort of patients at Mayo who exhibited pathological complete response (pCR) or no pCR to neoadjuvant chemotherapy (NAC). FIG. 14C) The correlation between FOXA1 expression level and the DNA mutational load in breast cancers from a cohort of patients at Mayo who exhibited pCR or no pCR to NAC. FIG. 14D) Microarray data analysis of the association of expression of FOXA1, CD3E, CD8A and GZMB in breast cancers of a cohort of 253 patients (NCT00455533; GSE41998) who exhibited pCR or no pCR to NAC.

FIG. 15. Expression of FOXA1 in urothelial carcinomas treated with anti-PD1 immunotherapy. FOXA1 IHC was performed using a FOXA1-specific antibody on the specimens from 22 cases of urothelial carcinomas treated with anti-PD1 immunotherapy. Low and high magnification of FOXA1 IHC images for each case and FOXA1 IHC scores are shown (see scoring details in Materials and Methods in Supplementary Information and in Table 4 (FIG. 18)).

FIG. 16. A hypothetical model deciphering FOXA1 overexpression-mediated inhibition of IFN signaling and anti-tumor immune response in cancer. Upon interferon (IFN) stimulation STAT1 and STAT2 proteins become phosphorylated, dimerized (STAT2/STAT1 heterodimer or STAT1/STAT1 homodimer), and translocate into nucleus to initiate the transcription of interferon-stimulated genes (ISGs) by binding to specific DNA elements (ISRE or GAS motifs) and promote anti-tumor immune response (Left). In cells with overexpression of FOXA1, however, FOXA1 binds to the STAT protein complex and impair ISG gene expression, thereby inhibiting tumor immunity in cancer (Right).

FIG. 17. Top 100 granule exocytosis genes negatively correlated genes (Table 1).

FIG. 18. FOXA1 IHC staining of urothelial carcinoma with anti-PD1 treatment (Table 4). FOXA1 staining data is shown in FIG. 16.

FIGS. 19A-19B. FOXA1 ASOs sensitize prostate cancer to anti-PD-L1 immunotherapy in mice. (FIG. 19A) Western blot analysis of Foxal protein in MyC-CaP mouse prostate cancer cells at 48 hours after transfection with control ASO (Con ASO), Foxal gene specific ASO1, or Foxa1 gene specific ASO2. Erk2 was used as a loading control. (FIG. 19B) Growth of subcutaneous MyC-CaP prostate tumors in wild type intact FVB male mice. Mice were treated with anti-PD-L1 or non-specific IgG (10 mg/kg) in combination with control ASO (Con ASO) (12.5 mg/kg), Foxal ASO1 (12.5 mg/kg), or Foxa1 ASO2 (12.5 mg/kg) for the indicated number of days. Data shown as means ±s.d. (n=6). Statistical significance was determined by two-way ANOVA (* P<0.05; ** P <0.01; *** P <0.0001).

### DETAILED DESCRIPTION

This document provides methods and materials involved in assessing and/or treating mammals (e.g., humans) having cancer. For example, the methods and materials provided herein can be used to determine whether or not a mammal having cancer is likely to be responsive to a particular cancer treatment (e.g., one or more cancer immunotherapies and/or one or more cancer chemotherapies). In some cases, the methods and materials provided herein also can include administering one or more cancer treatments to a mammal having cancer to treat the mammal (e.g., one or more cancer treatments that is/are selected based, at least in part, on whether or not the mammal is likely to be responsive to a particular cancer treatment).

Any appropriate mammal having a cancer can be assessed and/or treated as described herein. Examples of mammals having a cancer that can be assessed and/or treated as described herein include, without limitation, humans, non-human primates (e.g., monkeys), dogs, cats, horses, cows, pigs, sheep, mice, and rats. In some cases, a human having a cancer can be assessed and/or treated as described herein.

When assessing and/or treating a mammal (e.g., a human) having a cancer as described herein, the cancer can be any type of cancer. In some cases, a cancer can be a blood cancer. In some cases, a cancer can include one or more solid tumors. In some cases, a cancer can be a luminal cancer. In some cases, a cancer can be a primary cancer. In some cases, a cancer can be a metastatic cancer. Examples of cancers that can be assessed and/or treated as described herein include, without limitation, prostate cancers (e.g., prostate adenocarcinoma), breast cancers (e.g., breast invasive carcinomas and TNBCs), bladder cancers (e.g., bladder urothelial carcinomas), lung cancers (e.g., lung adenocarcinomas, lung squamous cell carcinomas, and mesotheliomas), liver cancers (e.g., liver hepatocellular carcinomas), cervical cancers (e.g., cervical squamous cell carcinomas and endocervical adenocarcinomas), bile duct cancers (e.g., cholangiocarcinomas), colon cancers (colon adenocarcinomas), rectal cancers (e.g., rectum adenocarcinomas), pancreatic cancers (e.g., pancreatic adenocarcinomas), uterine cancers (e.g., uterine corpus endometrial carcinomas and uterine carcinosarcomas), head and neck cancers (e.g., head and neck squamous cell carcinomas), testicular cancers (e.g., testicular germ cell tumors), ovarian cancers (e.g., ovarian serous cystadenocarcinoma), thyroid cancers (e.g., thyroid carcinomas), bone cancers (e.g., sarcomas), skin cancers (e.g., skin cutaneous melanoma), adrenal gland cancers (e.g., adrenocortical carcinomas, pheochromocytoma, and paraganglioma), kidney cancers (e.g., kidney renal clear cell carcinoma, kidney renal papillary cell carcinoma, and kidney chromophobes), lymphomas (e.g., lymphoid neoplasm diffuse large B-cell lymphoma), thymus cancers (e.g., thymoma), brain cancers (e.g., brain lower grade glioma and glioblastoma multiforme), leukemias (acute myeloid leukemia), and cancers of the eye (e.g., uveal melanoma).

In some cases, the methods described herein can include identifying a mammal (e.g., a human) as having a cancer. Any appropriate method can be used to identify a mammal as having a cancer. For example, imaging techniques and/or biopsy techniques can be used to identify mammals (e.g., humans) having cancer. A mammal having cancer can be assessed to determine whether or not the cancer is likely to respond to a particular cancer treatment (e.g., one or more cancer immunotherapies and/or one or more cancer chemotherapies). In some cases, a sample (e.g., a sample containing one or more cancer cells) obtained from a mammal having cancer can be assessed for the presence, absence, or level of FOXA1 polypeptide expression. As described herein, the level of FOXA1 polypeptide expression in a sample obtained from a mammal having a cancer can be used to determine whether or not the mammal is likely to respond to a particular cancer treatment. For example, the presence of an increased level of FOXA1 polypeptide expression in a sample obtained from a mammal having cancer can indicate that the mammal is not likely to be responsive to one or more cancer immunotherapies and/or one or more cancer chemotherapies. The term “increased level” as used herein with respect to FOXA1 polypeptide expression refers to any level that is higher than a reference level of FOXA1 polypeptide expression. The term “reference level” as used herein with respect to FOXA1 polypeptide expression refers to the level of FOXA1 polypeptide expression typically observed in a sample (e.g., a control sample) from one or more healthy mammals (e.g., mammals that do not have a cancer). Control samples can include, without limitation, samples from normal (e.g., healthy) mammals, primary cell lines derived from normal (e.g., healthy mammals), and non-tumorigenic cells lines. In some cases, an increased level of FOXA1 polypeptide expression can be a level that is at least >1 (e.g., at least 2, at least 5, at least 10, at least 15, at least 20, at least 25, at least 35, or at least 50) fold greater relative to a reference level of FOXA1 polypeptide expression. In some cases, when control samples have an undetectable level of FOXA1 polypeptide expression, an increased level can be any detectable level of FOXA1 polypeptide expression. It will be appreciated that levels from comparable samples are used when determining whether or not a particular level is an increased level.

Any appropriate sample from a mammal (e.g., a human) having cancer can be assessed as described herein (e.g., for the presence, absence, or level of FOXA1 polypeptide expression). In some cases, a sample can be a biological sample. In some cases, a sample can contain one or more cancer cells. In some cases, a sample can contain one or more biological molecules (e.g., nucleic acids such as DNA and RNA, polypeptides, carbohydrates, lipids, hormones, and/or metabolites). Examples of samples that can be assessed as described herein include, without limitation, tissue samples (e.g., tumor tissues such as those obtained by biopsy), fluid samples (e.g., whole blood, serum, plasma, urine, and saliva), cellular samples (e.g., buccal samples), and samples from surgery. A sample can be a fresh sample or a fixed sample (e.g., a formaldehyde-fixed sample or a formalin-fixed sample). In some cases, a sample can be a processed sample (e.g., an embedded sample such as a paraffin or OCT embedded sample). In some cases, one or more biological molecules can be isolated from a sample. For example, nucleic acid (e.g., DNA and RNA such as messenger RNA (mRNA)) can be isolated from a sample and can be assessed as described herein. For example, one or more polypeptides can be isolated from a sample and can be assessed as described herein.

Any appropriate method can be used to detect the presence, absence, or level of FOXA1 polypeptide expression within a sample (e.g., a sample containing one or more cancer cells) obtained from a mammal (e.g., a human). In some cases, the presence, absence, or level of FOXA1 polypeptide expression within a sample can be determined by detecting the presence, absence, or level of FOXA1 polypeptides in the sample. For example, immunoassays (e.g., immunohistochemistry (IHC) techniques and western blotting techniques), mass spectrometry techniques (e.g., proteomics-based mass spectrometry assays or targeted quantification-based mass spectrometry assays), enzyme-linked immunosorbent assays (ELISAs), radio-immunoassays, and immunofluorescent cytochemistry (IFC) can be used to determine the presence, absence, or level of FOXA1 polypeptides in a sample. When an immunoassay is used to determine the presence, absence, or level of FOXA1 polypeptides in a sample, the immunoassay can include using any appropriate anti-FOXA1 polypeptide antibody. Examples of representative anti-FOXA1 polypeptide antibodies that can be used in an immunoassay (e.g., IFC or ELISA) to determine the presence, absence, or level of FOXA1 polypeptides in a sample include, without limitation, Abcam # ab23738, Santa Cruz Biotechnology # sc-101058, Abcam # ab170933, Abcam # ab170933, Abcam # ab23738, Abcam # ab55178, Abcam # ab236011, Abcam # ab5089, Abcam # ab151522, Abcam # ab173287, Abcam # ab240935, Abcam # ab99892, Abcam # ab218885, Abcam # ab197235, Abcam # ab249749, Abcam # ab226380, Abcam # ab218201, Abcam # ab227785, and Abcam # ab196908. In some cases, the presence, absence, or level of FOXA1 polypeptide expression within a sample can be determined by detecting the presence, absence, or level of mRNA encoding a FOXA1 polypeptide in the sample. For example, polymerase chain reaction (PCR)-based techniques such as quantitative RT-PCR techniques, gene expression panel (e.g., next generation sequencing (NGS) such as RNA-seq), in situ hybridization, and microarray gene expression profiling can be used to determine the presence, absence, or level of mRNA encoding a FOXA1 polypeptide in the sample.

In some cases, a mammal having cancer and assessed as described herein (e.g., to determine whether or not the cancer is likely to respond to a particular cancer treatment based, at least in part, on the level of FOXA1 polypeptide expression), can be administered or instructed to self-administer any one or more (e.g., 1, 2, 3, 4, 5, 6, or more) cancer treatments, where the one or more cancer treatments are effective to treat the cancer within the mammal. For example, a mammal having cancer can be administered or instructed to self-administer any one or more cancer treatments that is/are selected based, at least in part, on whether or not the mammal is likely to be responsive to a particular cancer treatment (e.g., based, at least in part, on the level of FOXA1 polypeptide expression). In some cases, the level of FOXA1 polypeptide expression within a sample (e.g., a sample containing one or more cancer cells) obtained from a mammal can be used to determine whether or not the mammal is likely to be responsive to a particular cancer treatment. For example, the level of FOXA1 polypeptide expression in a sample can be used as a predictor of response to an immunotherapy (e.g., an anti-PD1 therapy and an anti-CTLA-4 therapy). For example, the presence or absence of an increased level of FOXA1 polypeptide expression in a sample can be used as a predictor of response to a chemotherapy (e.g., cisplatin).

When treating a mammal (e.g., a human) having cancer and identified as being likely to respond to one or more cancer immunotherapies and/or one or more cancer chemotherapies as described herein (e.g., based, at least in part, on the absence of an increased level of FOXA1 polypeptide expression), the mammal can be administered or instructed to self-administer any one or more (e.g., 1, 2, 3, 4, 5, 6, or more) cancer immunotherapies. For example, a mammal having cancer and identified as lacking an increased level of FOXA1 polypeptide expression in a sample (e.g., a sample obtained from the mammal) can be administered or instructed to self-administer any one or more cancer immunotherapies. A cancer immunotherapy can include administering any appropriate molecule(s) that can enhance an immune response against a cancer within a mammal. Examples of molecules that can enhance an immune response against a cancer within a mammal include, without limitation, polypeptides (e.g., antibodies such as monoclonal antibodies), T-cells (e.g., a chimeric antigen receptor (CAR) T-cells), immune checkpoint inhibitors (e.g., PD1 inhibitors, PD-L1 inhibitors, and CTLA-4 inhibitors), cancer vaccines, cytokines, immunomodulators, and adoptive transfer of tumor infiltrated lymphocytes (TILs). Examples of cancer immunotherapies that can be administered to a mammal having cancer and identified as being likely to be responsive to one or more cancer immunotherapies include, without limitation, pembrolizumab (formerly MK-3475 or lambrolizumab; e.g., KEYTRUDA), nivolumab (OPDIVO®), cemiplimab)(LIBTAY®), spartalizumab (PDR001), camrelizumab (SHR1210), sintilimab (IBI308), tislelizumab (BGB-A317), toripalimab (JS 001), AMP-224, AMP-514, atezolizumab (TECENTRIQ®), avelumab (BAVENCIO®), durvalumab (IMFINZI®), KN035, CK-301, AUNP12, CA-170, and BMS-986189.

When treating a mammal (e.g., a human) having cancer and identified as being likely to respond to one or more cancer immunotherapies and/or one or more cancer chemotherapies as described herein (e.g., based, at least in part, on the absence of an increased level of FOXA1 polypeptide expression), the mammal can be administered or instructed to self-administer any one or more (e.g., 1, 2, 3, 4, 5, 6, or more) cancer chemotherapies. For example, a mammal having cancer and identified as lacking an increased level of FOXA1 polypeptide expression in a sample (e.g., a sample obtained from the mammal) can be administered or instructed to self-administer any one or more cancer chemotherapies. A cancer chemotherapy can include administering any appropriate compound that is cytotoxic to one or more cancer cells within a mammal. Examples of compounds that are cytotoxic to one or more cancer cells within a mammal include, without limitation, alkylating agents, antimetabolites, anti-microtubule agents, topoisomerase inhibitors, and cytotoxic antibiotics. Examples of cancer chemotherapies that can be administered to a mammal having cancer and identified as being likely to be responsive to one or more cancer chemotherapies include, without limitation, actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, and vindesine.

When treating a mammal (e.g., a human) having cancer and identified as not being likely to respond to one or more cancer immunotherapies and/or one or more cancer chemotherapies as described herein (e.g., based, at least in part, on the presence of an increased level of FOXA1 polypeptide expression), the mammal can be administered or instructed to self-administer any one or more (e.g., 1, 2, 3, 4, 5, 6, or more) alternative cancer treatments (e.g., one or more cancer treatments that are not a cancer immunotherapy or a cancer chemotherapy). For example, a mammal having cancer and identified as having an increased level of FOXA1 polypeptide expression in a sample (e.g., a sample obtained from the mammal) can be administered or instructed to self-administer any one or more cancer treatments that are not a cancer immunotherapy or a cancer chemotherapy. An alternative cancer treatment can include any appropriate cancer treatment. Examples of alternative cancer treatments include, without limitation, surgery, radiation treatment, targeted therapies, hormone therapies, and stem cell transplants.

When treating a mammal (e.g., a human) having cancer and identified as not being likely to respond to one or more cancer immunotherapies and/or one or more cancer chemotherapies as described herein (e.g., based, at least in part, on the presence of an increased level of FOXA1 polypeptide expression), the mammal can be administered or instructed to self-administer any one or more (e.g., 1, 2, 3, 4, 5, 6, or more) inhibitors of a FOXA1 polypeptide, and, optionally, can be administered or instructed to self-administer any one or more (e.g., 1, 2, 3, 4, 5, 6, or more) cancer immunotherapies and/or one or more (e.g., 1, 2, 3, 4, 5, 6, or more) cancer chemotherapies. For example, a mammal having cancer and identified as having an increased level of FOXA1 polypeptide expression in a sample (e.g., a sample obtained from the mammal) can be administered or instructed to self-administer any one or more inhibitors of a FOXA1 polypeptide, and, optionally, can be administered or instructed to self-administer any one or more cancer immunotherapies and/or one or more cancer chemotherapies. In some cases, one or more inhibitors of a FOXA1 polypeptide can be administered to a mammal having cancer and identified as not being likely to respond to one or more cancer immunotherapies and/or one or more cancer chemotherapies to sensitize the cancer cells to one or more cancer immunotherapies, and, optionally one or more cancer immunotherapies can be administered to the mammal. In some cases, one or more inhibitors of a FOXA1 polypeptide can be administered to a mammal having cancer and identified as not being likely to respond to one or more cancer immunotherapies and/or one or more cancer chemotherapies to sensitize the cancer cells to one or more cancer chemotherapies, and, optionally one or more cancer chemotherapies can be administered to the mammal.

In some cases, one or more inhibitors of a FOXA1 polypeptide described herein can be administered to a mammal (e.g., a human) to alter (e.g., increase or decrease) the level of one or more interferons (IFNs) in one or more cancer cells within the mammal. For example, one or more inhibitors of a FOXA1 polypeptide provided herein can be administered to a mammal in need thereof (e.g., a human having cancer) as described herein to alter the amount of one or more IFNs in one or more cancer cells within the mammal by, for example, 10, 20, 30, 40, 50, 60, 70, 80, 90, 95, or more percent. An IFN can be any appropriate IFN (e.g., type I IFN, type II IFN, or type III IFN). An example of an IFN whose level can be increased in one or more cancer cells following administration of one or more inhibitors of a FOXA1 polypeptide provided herein can include, without limitation, an IFN-y polypeptide. An example of an IFN whose level can be decreased in one or more cancer cells following administration of one or more inhibitors of a FOXA1 polypeptide provided herein can include, without limitation, an IFN-a polypeptide.

In some cases, one or more inhibitors of a FOXA1 polypeptide described herein can be administered to a mammal (e.g., a human) to increase the amount of one or more lymphocytes (e.g., tumor-infiltrating lymphocytes) in the tumor microenvironment of a tumor within the mammal. For example, one or more inhibitors of a FOXA1 polypeptide provided herein can be administered to a mammal in need thereof (e.g., a human having cancer) as described herein to recruit one or more lymphocytes to the tumor microenvironment (e.g., to increase the amount of one or more lymphocytes in the tumor microenvironment) of a tumor within the mammal by, for example, 10, 20, 30, 40, 50, 60, 70, 80, 90, 95, or more percent. Examples of lymphocytes that can be increased in a tumor microenvironment following administration of one or more inhibitors of a FOXA1 polypeptide provided herein can include, without limitation, T cells such as CD4+T cells, CD8+T cells (e.g., CD8⁺ T effector cells (CD8⁺ T_{eff} cells)), CTLs (e.g., Tcs), and NK cells.

An inhibitor of a FOXA1 polypeptide can be any appropriate inhibitor of a FOXA1 polypeptide. An inhibitor of a FOXA1 polypeptide can be an inhibitor of FOXA1 polypeptide activity or an inhibitor of FOXA1 polypeptide expression. Examples of compounds that can reduce FOXA1 polypeptide activity include, without limitation, small molecules (e.g., a pharmaceutically acceptable salt of a small molecule) such as SNS-032 (BMS-387032; CAS No.: 345627-80-7), Ro 31-8220 (CAS No.: 138489-18-6), Aurora A Inhibitor I (CAS No.: 1158838-45-9), WZ8040 (CAS No.: 1214265-57-2), Dasatinib, Lapatinib, Saracatinib (AZD0530), JNK-IN-8 (CAS No.: 1410880-22-6), BI 2536 (CAS No.: 755038-02-9), Crenolanib (CP-868596), Herceptin, Momelotinib (CYT387), Dactolisib (BEZ235), PHA-793887 (CAS No.: 718630-59-2), NVP-BSK805 2HC1 (CAS No.: 1092499-93-8), Cediranib (AZD2171), PF-00562271 (CAS No.: 898044-15-0 (free base); CAS No.: 1279034-84-2 (HCl)), Alvocidib (Flavopiridol), AT7519 (CAS No.: 844442-38-2), Apicidin (CAS No.: 183506-66-3), or Volasertib (BI 6727). See, e.g., Wang et al., Int. J. Mol. Sci., 19:4123 (2018)). Examples of compounds that can reduce FOXA1 polypeptide expression and be used as described herein include, without limitation, nucleic acid molecules designed to induce RNA interference (RNAi) against FOXA1 polypeptide expression (e.g., a small interfering RNA (siRNA) molecule or a short hairpin RNA (shRNA) molecule), antisense molecules against FOXA1 polypeptide expression such as antisense oligoes (ASOs) against FOXA1 polypeptide expression, and miRNAs against FOXA1 polypeptide expression. In some cases, a nucleic acid molecule designed to induce RNAi against FOXA1 polypeptide expression or an antisense molecule against FOXA1 polypeptide expression can be a locked nucleic acid (LNA). For example, a nucleic acid molecule designed to induce RNAi against FOXA1 polypeptide expression or an antisense molecule against FOXA1 polypeptide expression can include one or more ribose moieties that are modified with an extra methylene bridge connecting the 2′ oxygen and 4′ carbon. In some cases, a nucleic acid molecule designed to induce RNAi against FOXA1 polypeptide expression or an antisense molecule against FOXA1 polypeptide expression can include a phosphorothioate (PS) backbone. For example, a nucleic acid molecule designed to induce RNAi against FOXA1 polypeptide expression or an antisense molecule against FOXA1 polypeptide expression can include at least one (e.g., one, two, three, four, five, six, seven, eight, nine, ten, eleven, or more) inter-nucleotide phosphorothioate bond. Examples of nucleic acid molecules designed to induce RNAi against FOXA1 polypeptide expression that can be used as described herein include, without limitation, nucleic acid comprising or consisting of the sequence GAGAGAAAAAAUCAACAGC (SEQ ID NO:1) and nucleic acid comprising or consisting of the sequence GCACUGCAAUACUCGCCUU (SEQ ID NO:2). Additional nucleic acid molecules designed to induce RNAi against FOXA1 polypeptide expression can be designed based on any appropriate nucleic acid encoding a FOXA1 polypeptide sequence. Examples of nucleic acids encoding a FOXA1 polypeptide sequence include, without limitation, those set forth in National Center for Biotechnology Information (NCBI) accession no. NM_004496.5, accession no. XM_017021246.1, accession no. NM_008259.4, accession no. XM_017314962.2, accession no. XM_006515483.1, accession no. XM_006515479.4, accession no. XM_006515481.2, and accession no. XM_0302465621 Examples of ASOs that can be used to reduce FOXA1 polypeptide expression as described herein include, without limitation, those set forth in Table 5.

TABLE 5
Exemplary ASOs against FOXA1

FOXA1 ASO#1
+A*+T*+C*A*G*C*A*T*G*G*C*C*A*T*+C*+C*+A (SEQ ID NO: 38)
FOXA1 ASO#2
+A*+C*+C*A*C*C*C*G*T*T*C*T*C*C*A*T*+C*+A*+A (SEQ ID NO: 39)
FOXA1 ASO#3
+A*+C*+T*C*G*C*C*T*T*A*C*G*G*C*T*C*T*+A*+C*+G (SEQ ID NO: 40)
FOXA1 ASO#4
+C*+C*+A*T*T*T*T*A*A*T*C*A*T*T*G*C*C*A*T*C*+G*+T*+G (SEQ ID NO: 41)
Foxa1 ASO1:
+G*+G*+T*A*G*C*G*C*C*A*T*A*A*G*G*A*G*+A*+G*+T (SEQ ID NO: 42)
Foxa1 ASO2:
+T*+G*+G*A*T*G*G*C*C*A*T*C*G*+T*+G*+A (SEQ ID NO: 43)
+indicates that the nucleotide immediately following the “+” symbol is a LNA in which the ribose moiety is modified with an extra methylene bridge connecting the 2′ oxygen and 4′ carbon
*indicates that the nucleotide immediately prior to the “*” symbol has a PS backbone

Any appropriate method can be used to administer one or more inhibitors of a FOXA1 polypeptide to a mammal (e.g., a mammal having cancer). In some cases, an inhibitor of a FOXA1 polypeptide can be administered directly to a mammal. In some cases, one or more vectors (e.g., one or more expression vectors or one or more viral vectors such a retroviral vector, a lentiviral vector, a measles viral vector, or an oncolytic viral vector such as herpes simplex virus viral vector) containing (e.g., engineered to contain) nucleic acid encoding an inhibitor of a FOXA1 polypeptide can be administered to a mammal. In some cases, one or more viral particles containing (e.g., engineered to contain) nucleic acid encoding an inhibitor of a FOXA1 polypeptide can be administered to a mammal.

When nucleic acid encoding an inhibitor of a FOXA1 polypeptide is contained in a viral particle, the viral particle can be any appropriate viral particle. A viral particle described herein (e.g., a viral particle containing nucleic acid encoding an inhibitor of a FOXA1 polypeptide) can include viral components (e.g., genetic material (e.g., a viral genome), a capsid, and/or an envelope) from any appropriate virus. A virus can be an infectious virus or an oncolytic virus. A virus can be a chimeric virus. A virus can be a recombinant virus. In some cases, a viral particle can include viral components from the same virus. In some cases, a viral particle can be a recombinant viral particle. For example, a recombinant viral particle can include viral components from different viruses (e.g., two or more different viruses). Examples of viruses from which viral components can be obtained include, without limitation, retroviruses, (e.g., lentiviruses), measles viruses, and oncolytic viruses such as herpes simplex viruses.

In some cases, a viral particle described herein (e.g., a viral particle containing nucleic acid encoding an inhibitor of a FOXA1 polypeptide) can be used to target one or more cancer cells within a mammal having cancer. For example, a viral particle described herein can be used to target cancer cells presenting an antigen (e.g., a tumor antigen) associated with a particular cancer. Examples of antigens associated with a particular cancer include, without limitation, CD19 (associated with B cell lymphomas, acute lymphoblastic leukemia (ALL), and chronic lymphocytic leukemia (CLL)), AFP (associated with germ cell tumors and/or hepatocellular carcinoma), CEA (associated with bowel cancer, lung cancer, and/or breast cancer), CA-125 (associated with ovarian cancer), MUC-1 (associated with breast cancer), ETA (associated with breast cancer), and MAGE (associated with malignant melanoma).

In some cases, when treating a mammal (e.g., a human) having cancer as described herein, the treatment can be effective to reduce the number of cancer cells present within a mammal. For example, the size (e.g., volume) of one or more tumors present within a mammal can be reduced using the materials and methods described herein. In some cases, the materials and methods described herein can be used to reduce the size of one or more tumors present within a mammal having cancer by, for example, 10, 20, 30, 40, 50, 60, 70, 80, 90, 95, or more percent. In some cases, the size (e.g., volume) of one or more tumors present within a mammal does not increase.

In some cases, when treating a mammal (e.g., a human) having cancer as described herein, the treatment can be effective to improve survival of the mammal. For example, disease-free survival (e.g., relapse-free survival) can be improved using the materials and methods described herein. For example, progression-free survival can be improved using the materials and methods described herein. In some cases, the materials and methods described herein can be used to improve the survival of a mammal having cancer by, for example, 10, 20, 30, 40, 50, 60, 70, 80, 90, 95, or more percent.

The invention will be further described in the following examples, which do not limit the scope of the invention described in the claims.

### EXAMPLES

### Example 1

### FOXA1 Suppresses Cancer Immunity Independently of DNA Binding Activity

Infiltration of lymphocytes in tumors is an essential step in immune attack of cancer cells. Indeed, the abundance of tumor-infiltrating lymphocytes (TILs) is a valuable prognostic factor for both chemotherapy and immune checkpoint inhibitor (ICI) therapy (Adams et al., J Clin Oncol 32:2959-2966 (2014); Loi et al., Ann Oncol 25:1544-1550 (2014); and Denkert et al., J Clin Oncol 28:105-113 (2010)). Cytotoxic lymphocytes (CTL), mainly cytotoxic T (Tc) and natural killer (NK) cells utilize granule exocytosis as a common mechanism to destroy cancer cells by expressing and releasing the pore forming proteins including perforin 1 (PRF 1), granule-associated enzymes (granzymes (GZMs)) and natural killer cell granule protein 7 (NKG7) (Martinez-Lostao, Clinical Cancer Research 21:5047-5056 (2015)). Prostate and breast cancer are generally immunologically “cold.”

Results

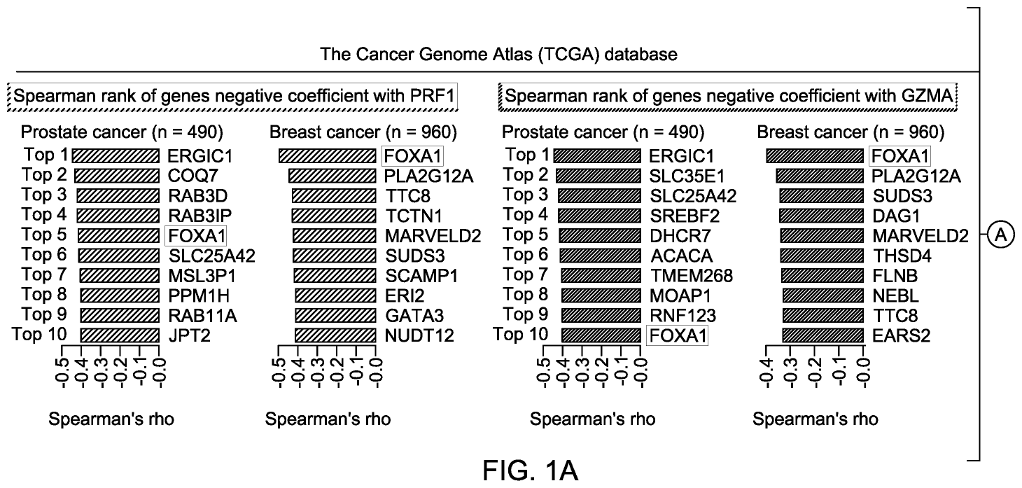

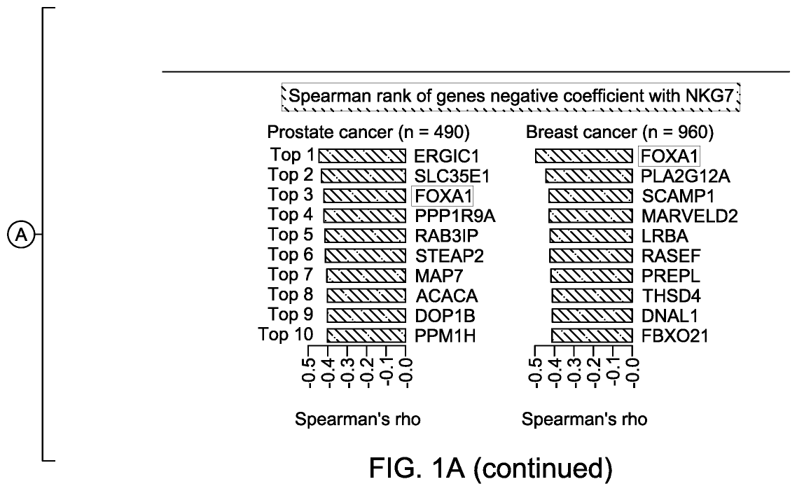

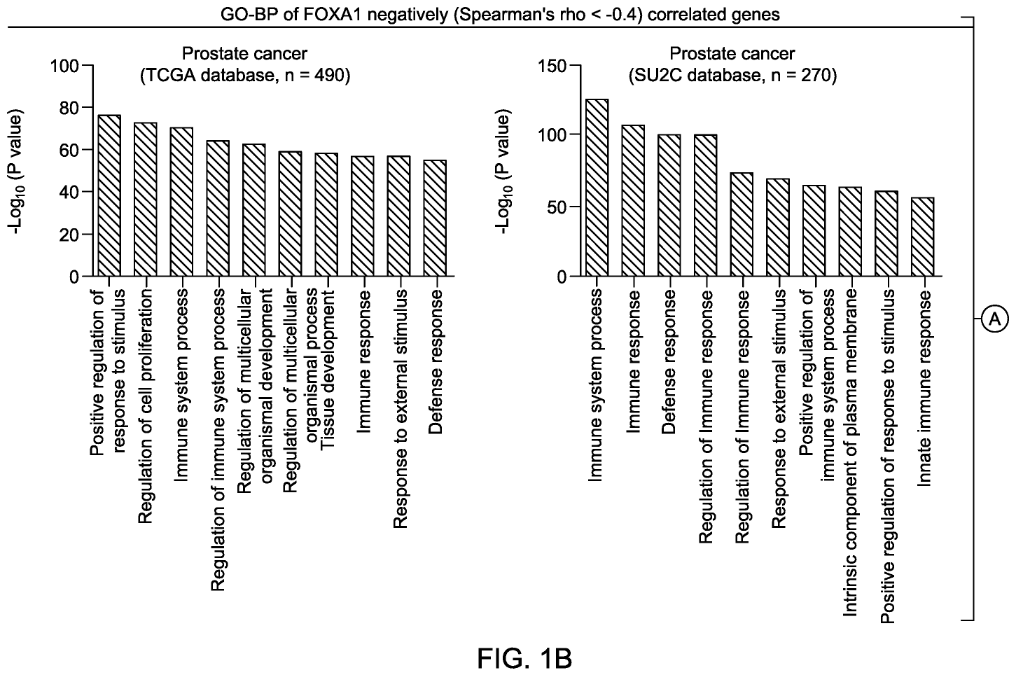

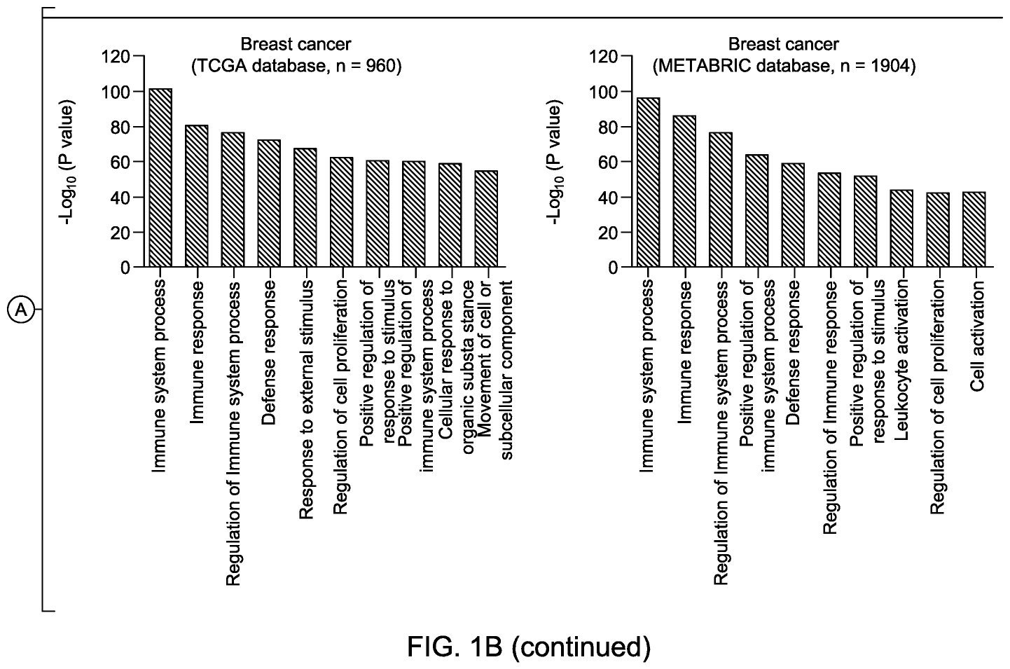

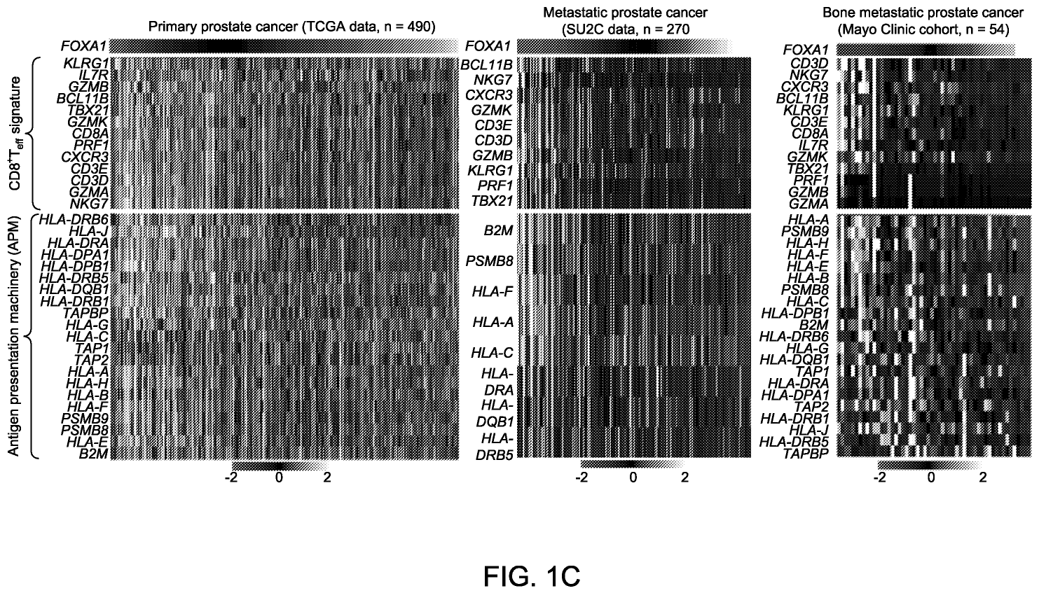

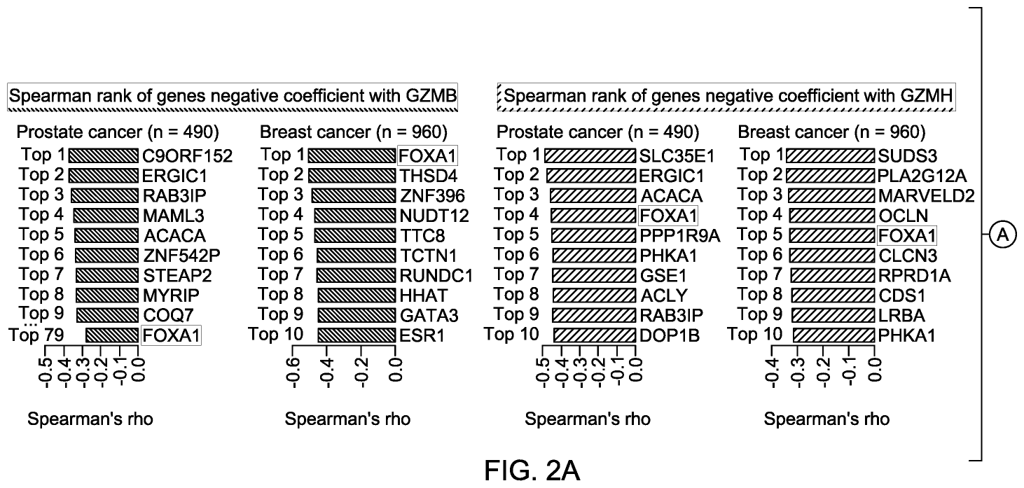

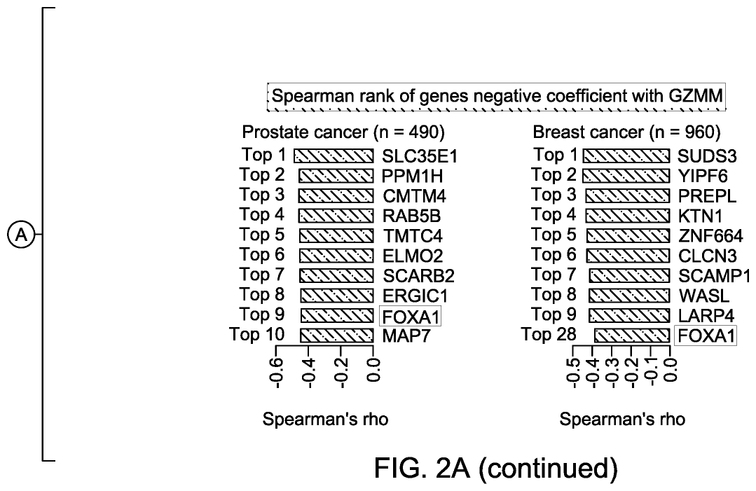

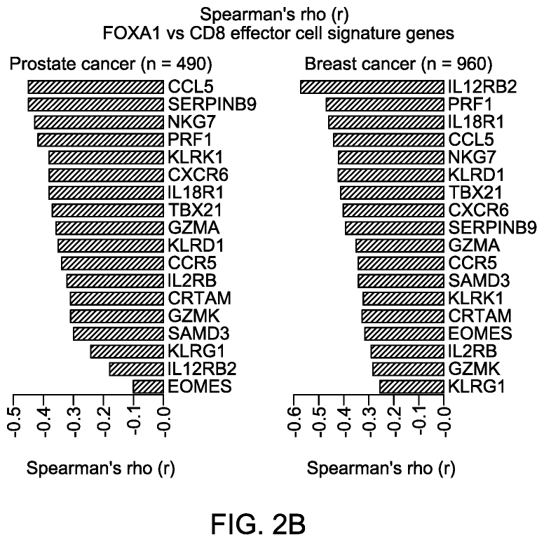

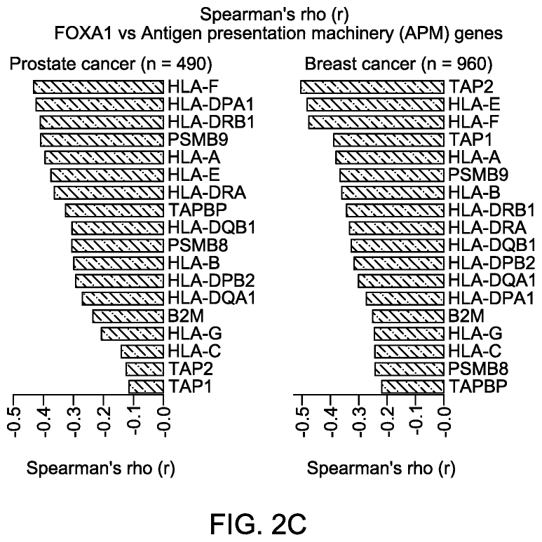

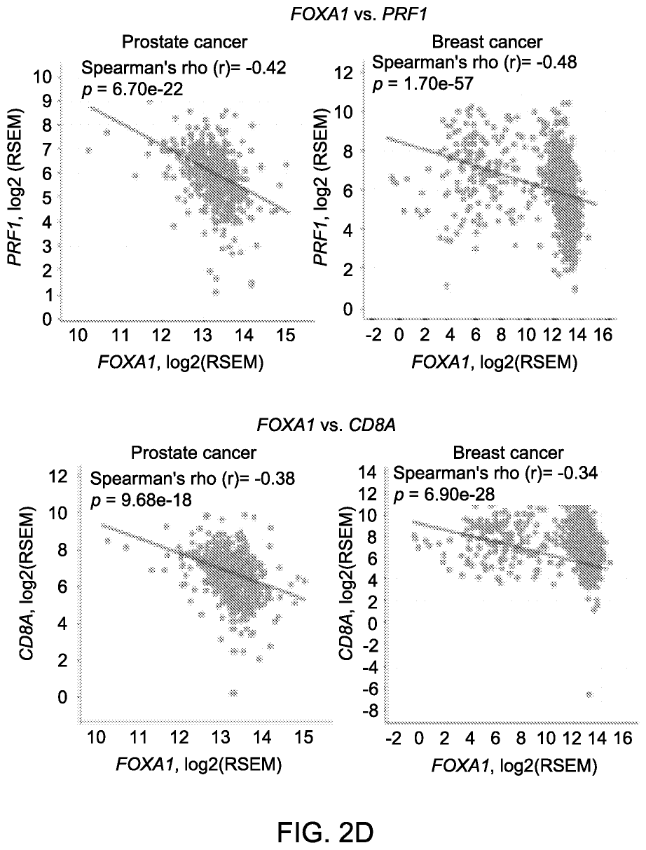

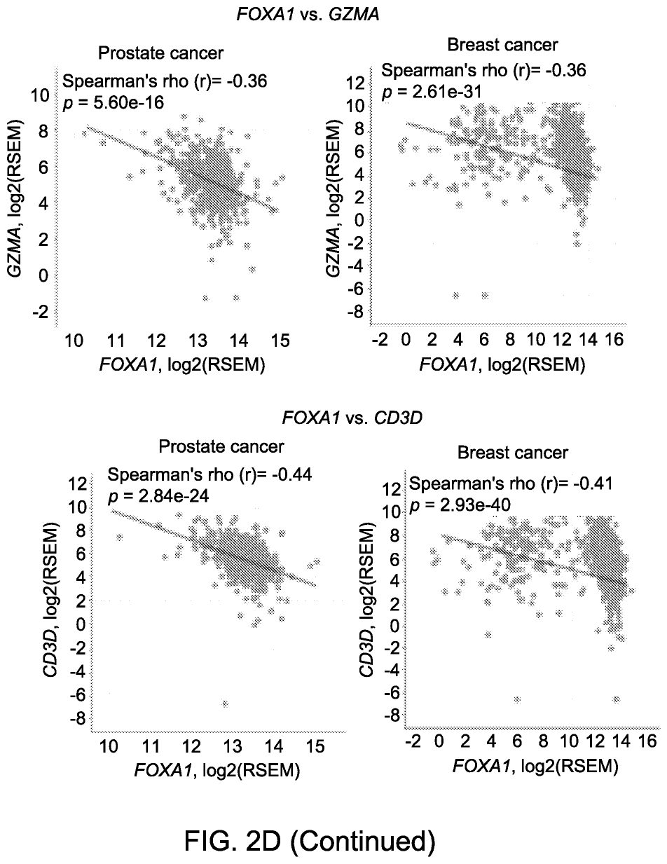

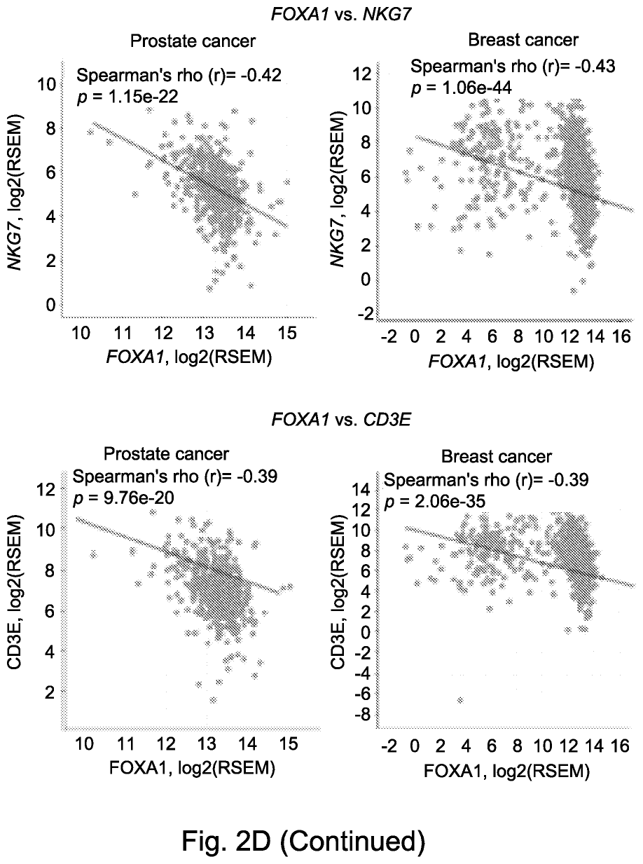

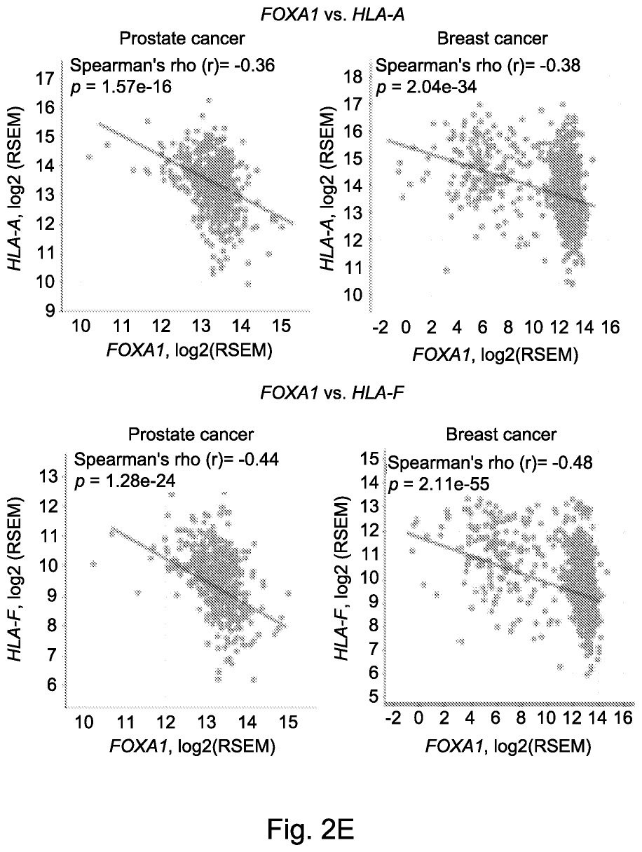

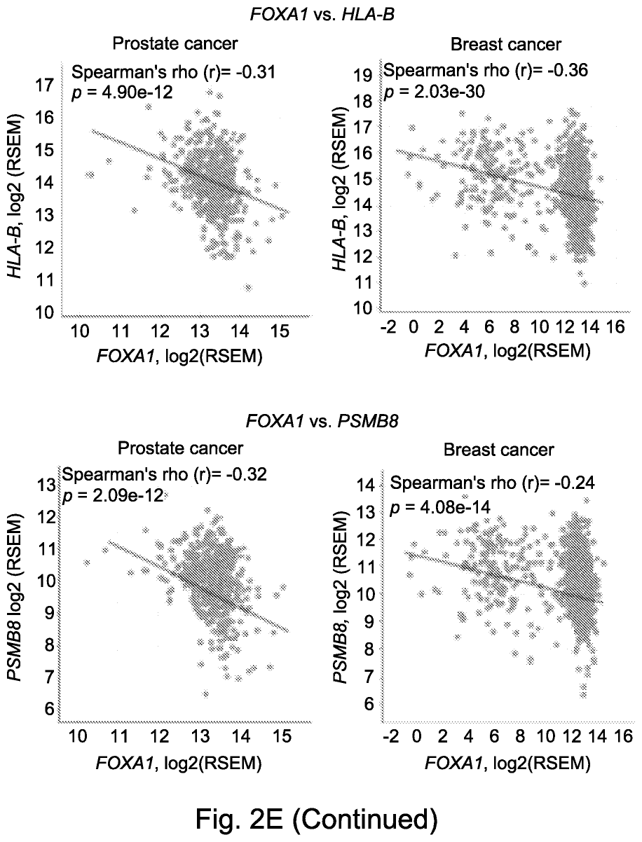

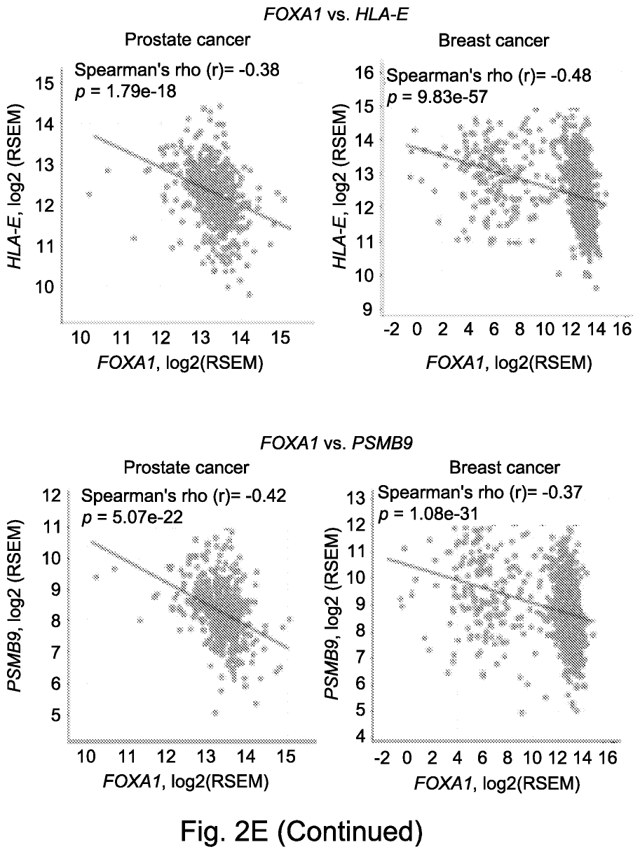

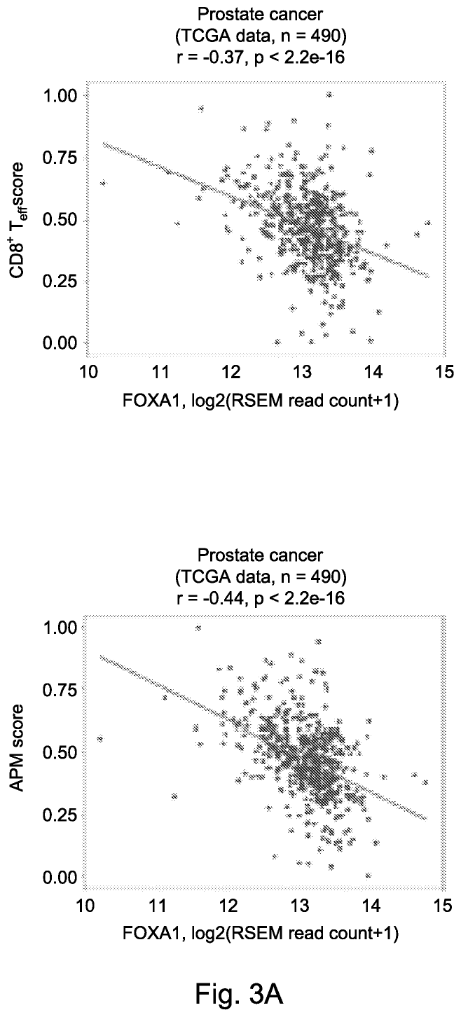

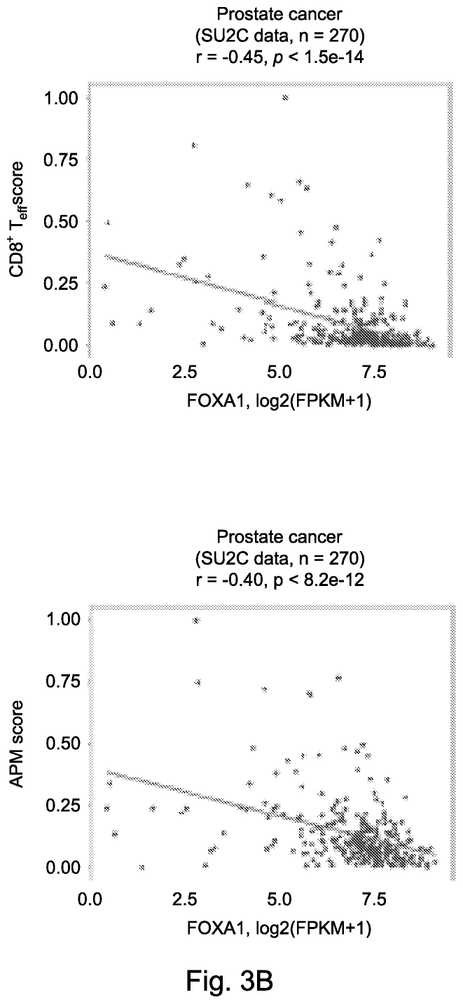

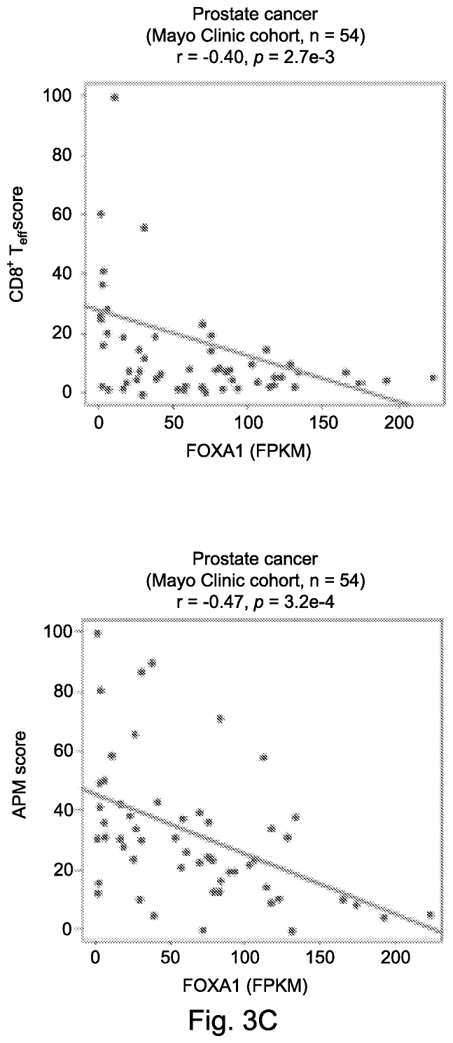

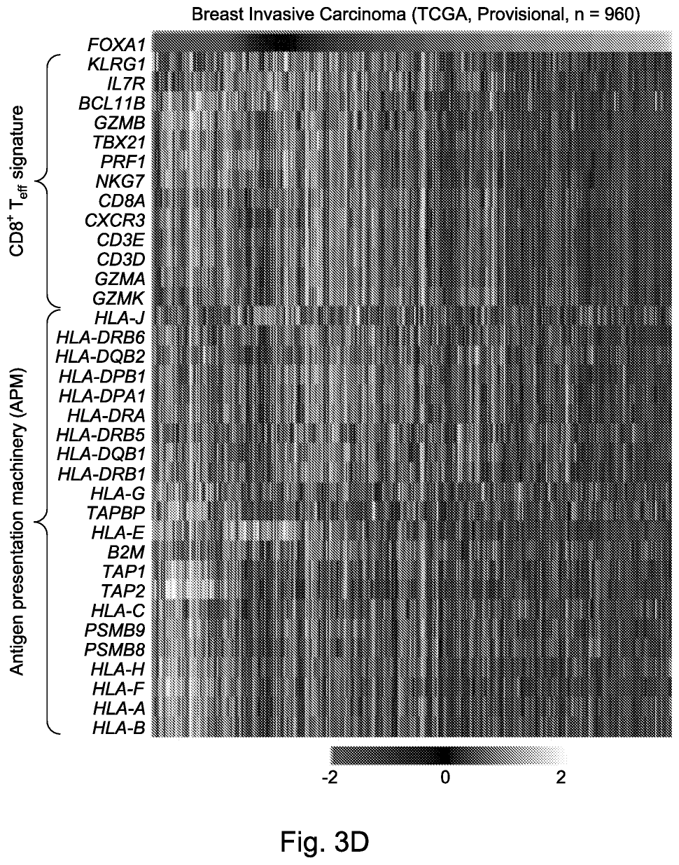

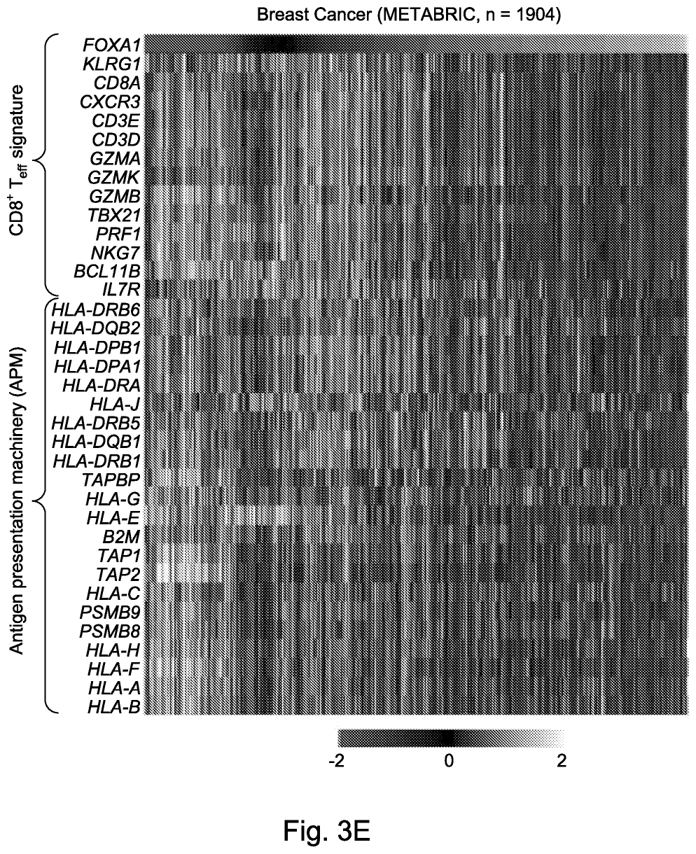

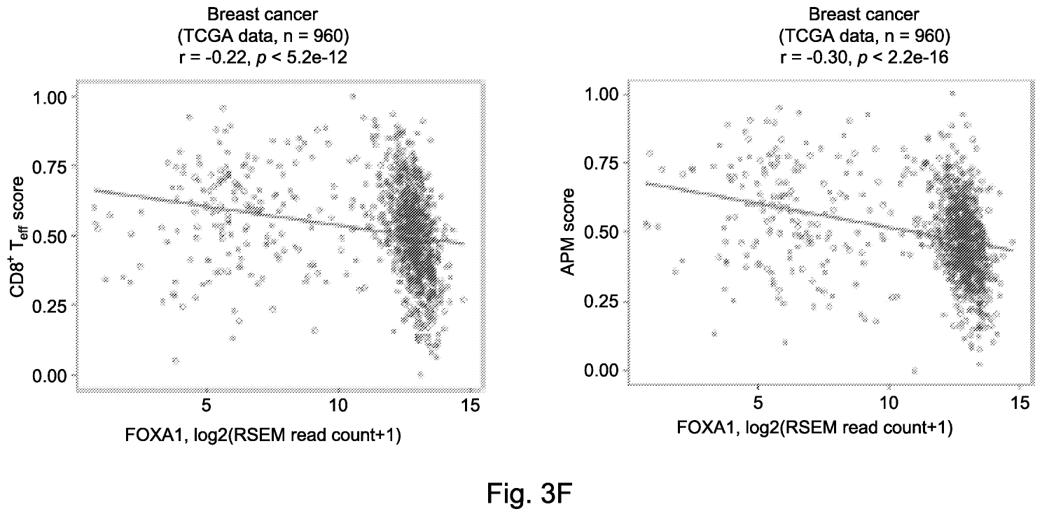

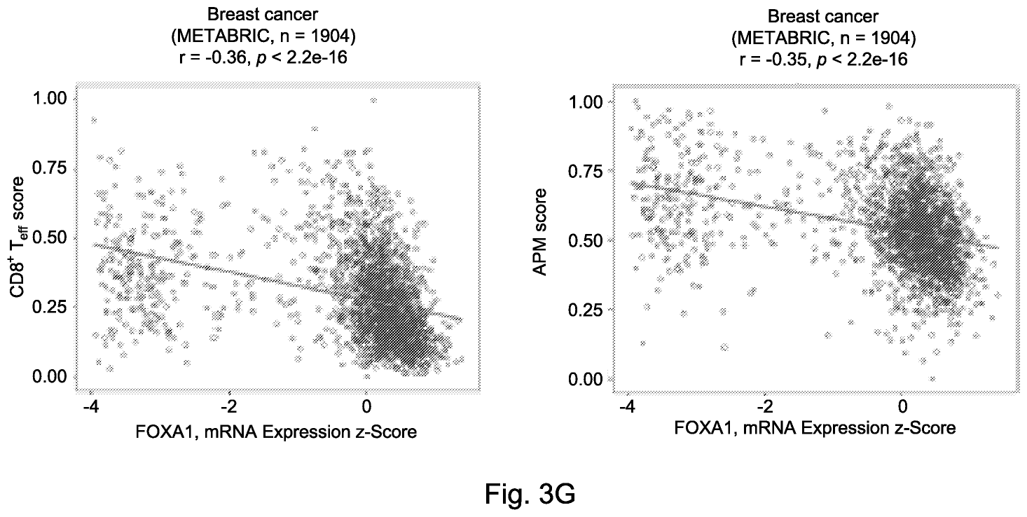

To identity the molecules and signaling pathways that contribute to immune evasion by blocking tumor infiltration of CTLs in prostate and breast cancers, meta-analysis of The Cancer Genome Atlas (TCGA) RNA-seq datasets was performed to search for genes that are negatively correlated with expression of the granule exocytosis genes PRF 1, GZMA and NKG7. It was demonstrated that among the top 10 genes negatively correlated with expression of PRF 1, GZMA and NKG7, FOXA1 is the only one gene commonly expressed in prostate and breast cancer (FIG. 1A). Similar relationships with FOXA1 expression were also observed for other GZM genes (except GZMB in prostate cancer and GZMM in breast cancer) (FIG. 2A). Gene Ontology Biological Process (GO-BP) analysis further revealed that immune response and regulatory signaling genes are among the top 10 pathways that are negatively correlated with FOXA1 level in both prostate and breast cancer patient specimens (FIG. 1B). These data suggest that FOXA1 might be an important suppressor of cancer immunity. In support of this notion, it was demonstrated that FOXA1 expression is negatively corrected with the mRNA level of CD8⁺ T effector cell (CD8⁺ T_{eff}) signature genes in different prostate and breast cancer cohorts (FIG. 1C, and FIGS. 2 and 3).

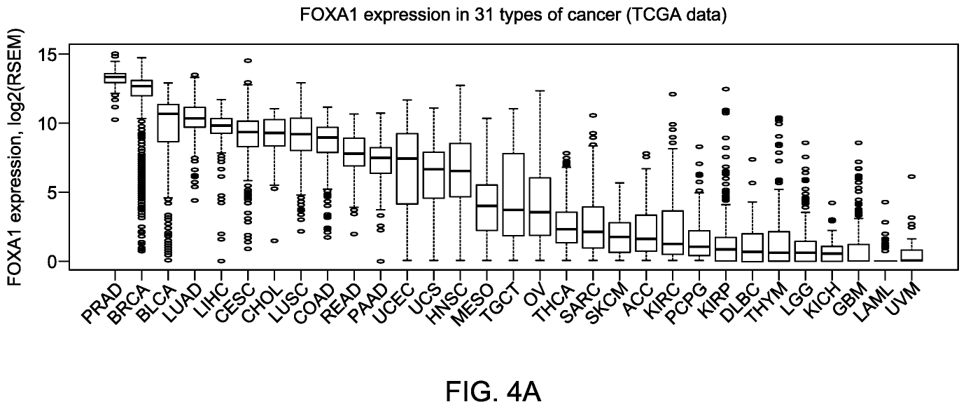

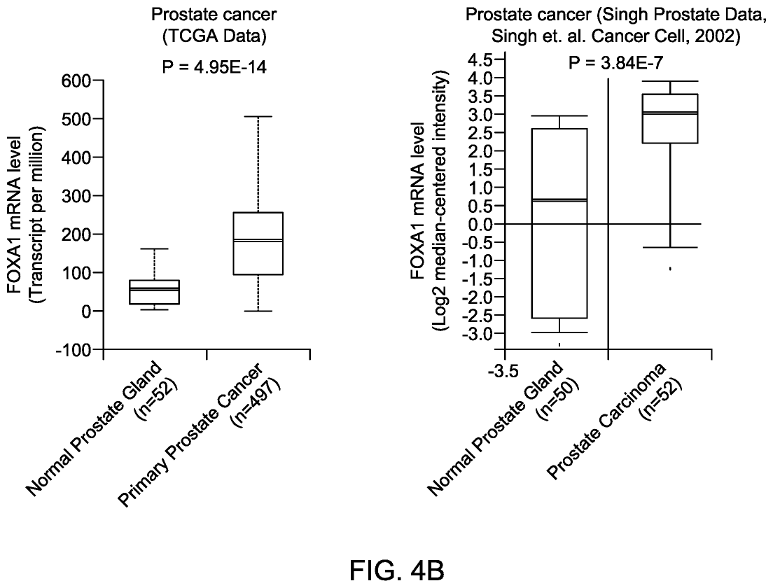

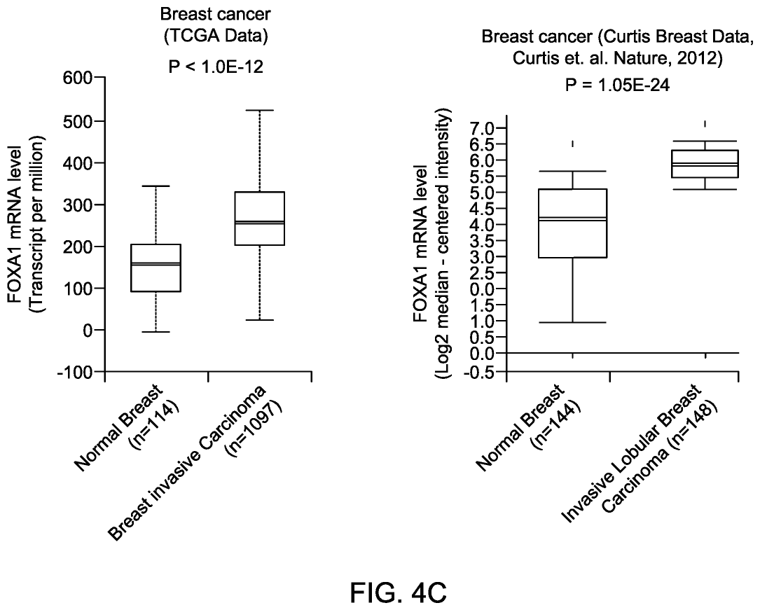

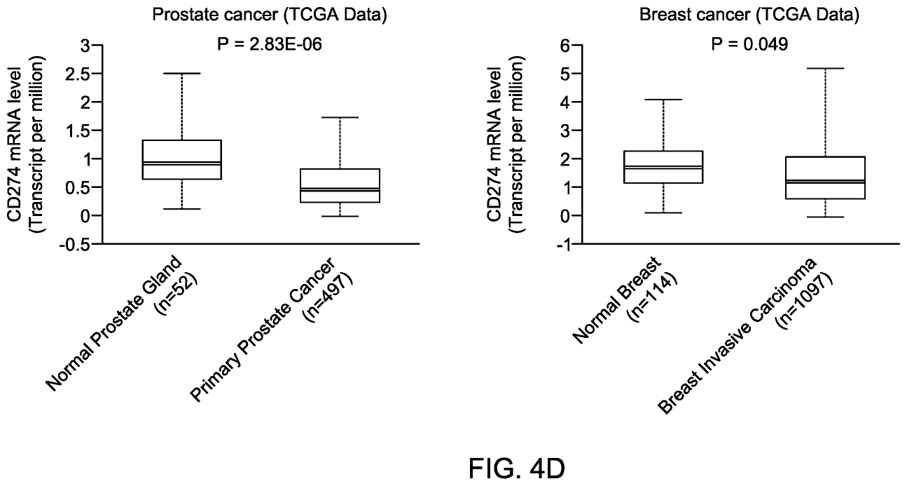

Presentation of cancer-specific neoantigens is a factor affecting Tc cell activity and ICI therapy efficacy. This step is governed by class I human leukocyte antigen (HLA) or major histocompatibility complex (MHC) that presents intra-cellular peptides on the cell surface for recognition by T cell receptors. FOXA1 level was unanimously inversely associated with the expression of antigen presentation machinery (APM) genes (FIG. 1C, FIG. 2, and FIG. 3). Among the 31 cancer types in TCGA datasets, the immune-insensitive cancers such as prostate adenocarcinoma and breast cancer are the top 2 malignancies that express the highest level of FOXA1 whereas FOXA1 expression was very low in the immune-sensitive tumor types such as melanoma (FIG. 4A). FOXA1 mRNA level was highly upregulated in prostate and breast cancer tissues compared with normal tissues (FIG. 4, B and C). Surprisingly, the CD27 4 (PD-L1) mRNA level was lower in both prostate and breast cancer compared with the normal tissues (FIG. 4D), indicating that getaway from the immune surveillance may not be primarily attributed to the increased expression of PD-L1 in most prostate and breast cancer patients. These data obtained from clinical specimens suggest that FOXA1 could be an important immune suppressor in prostate and breast cancer.

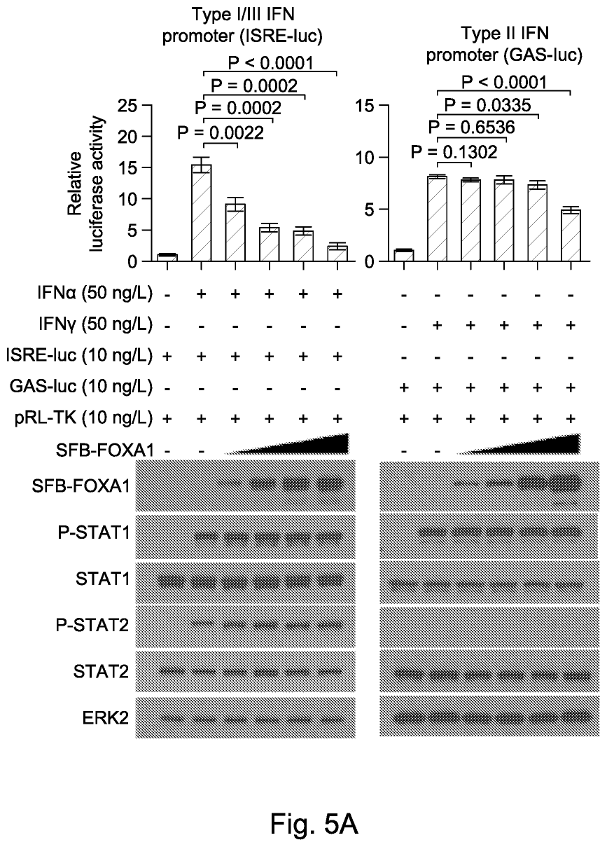

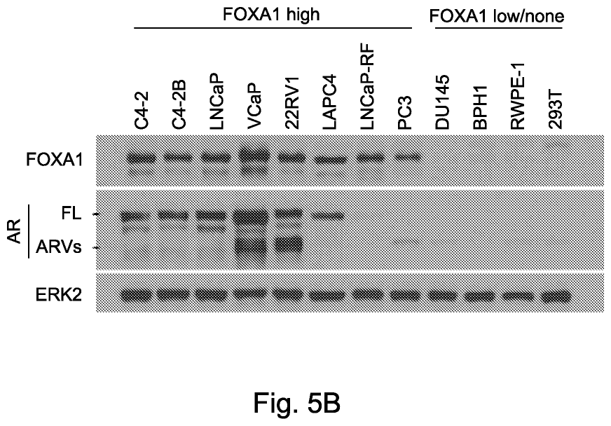

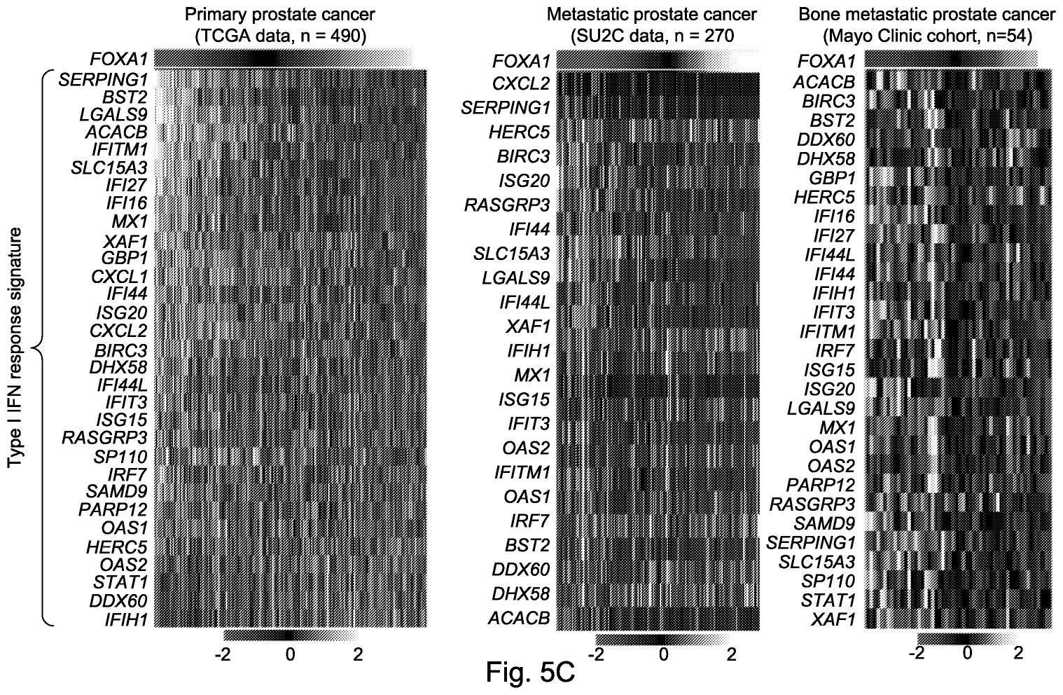

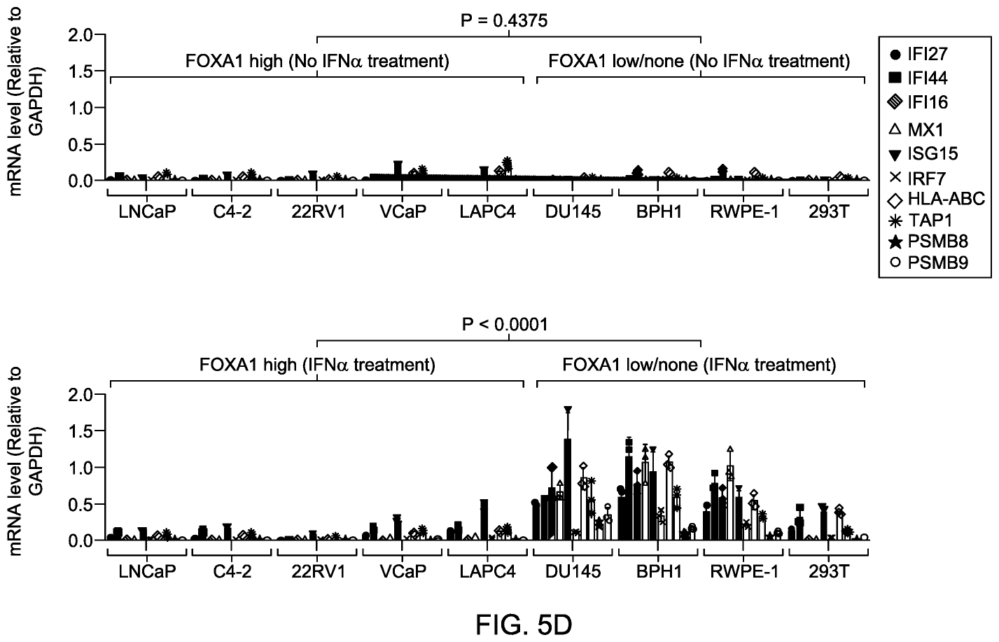

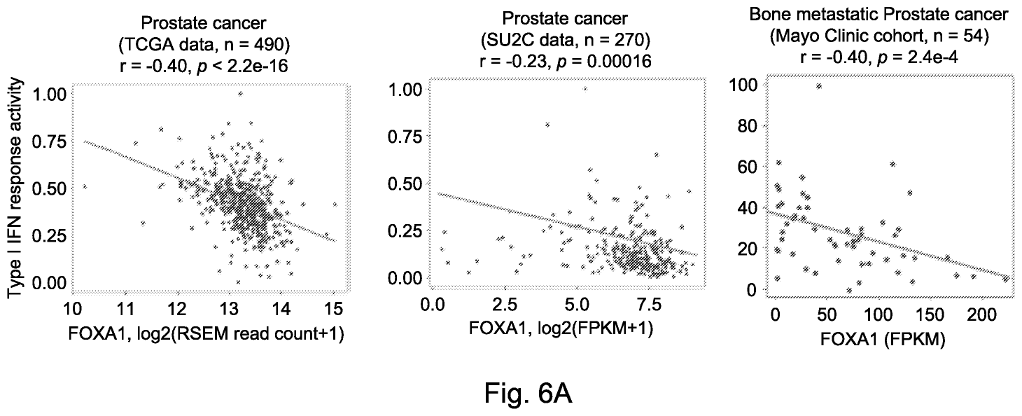

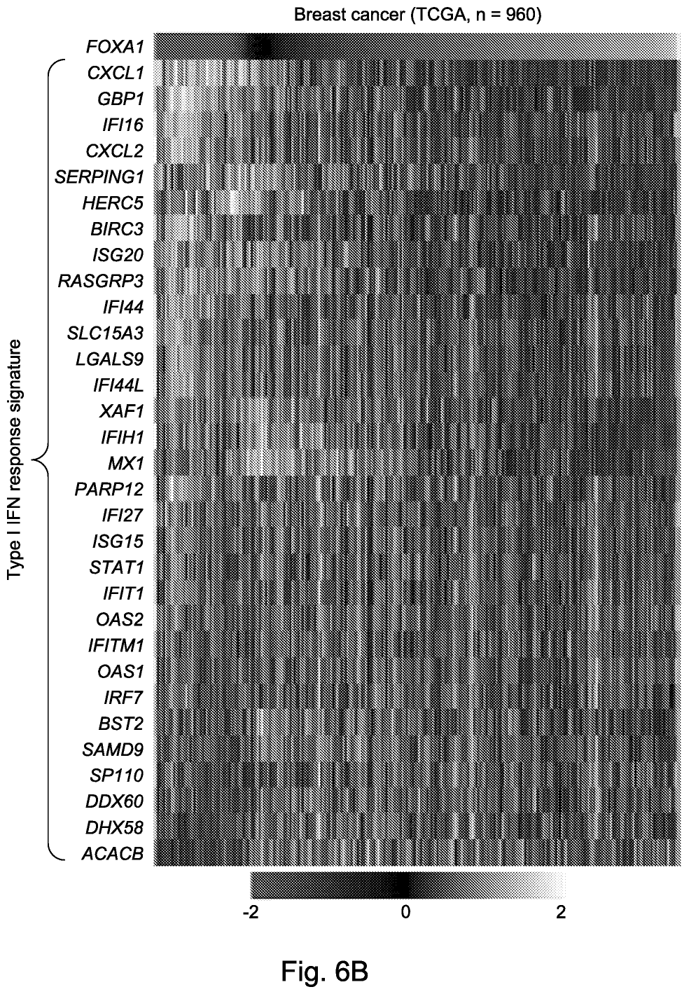

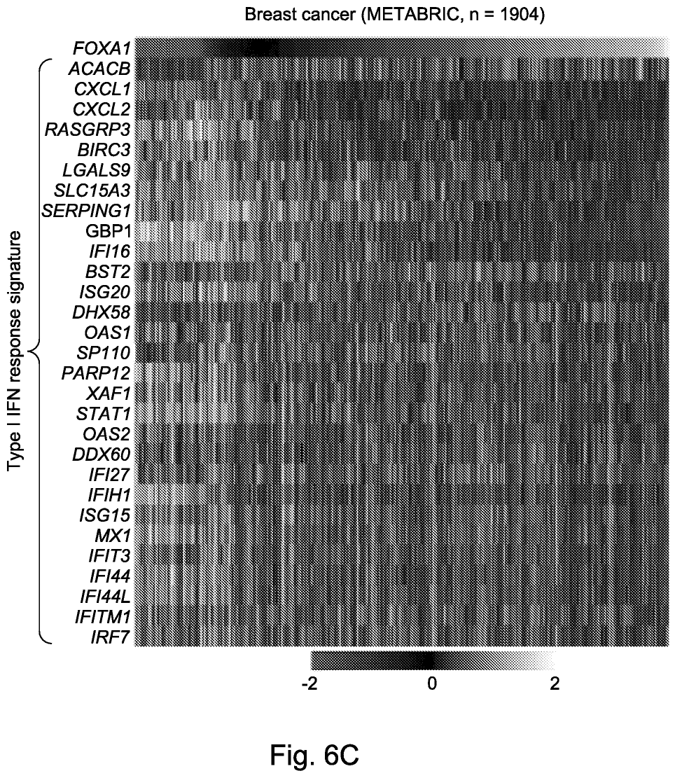

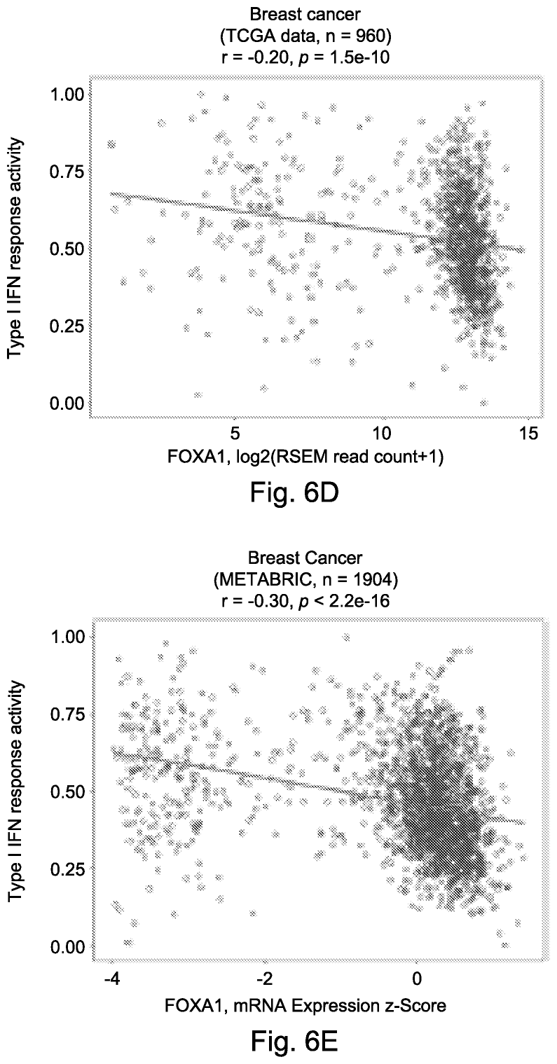

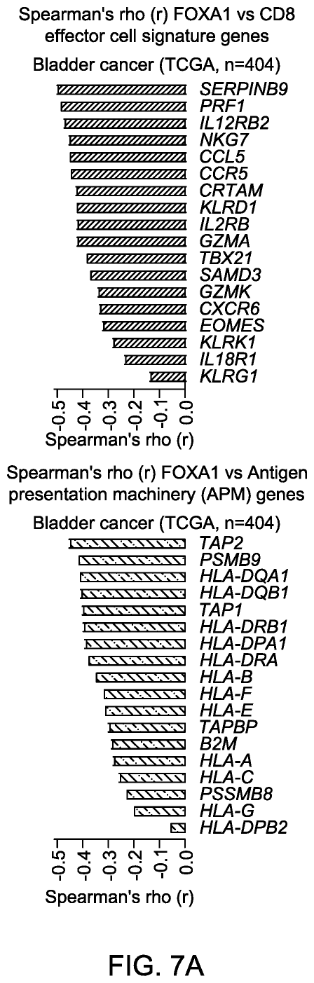

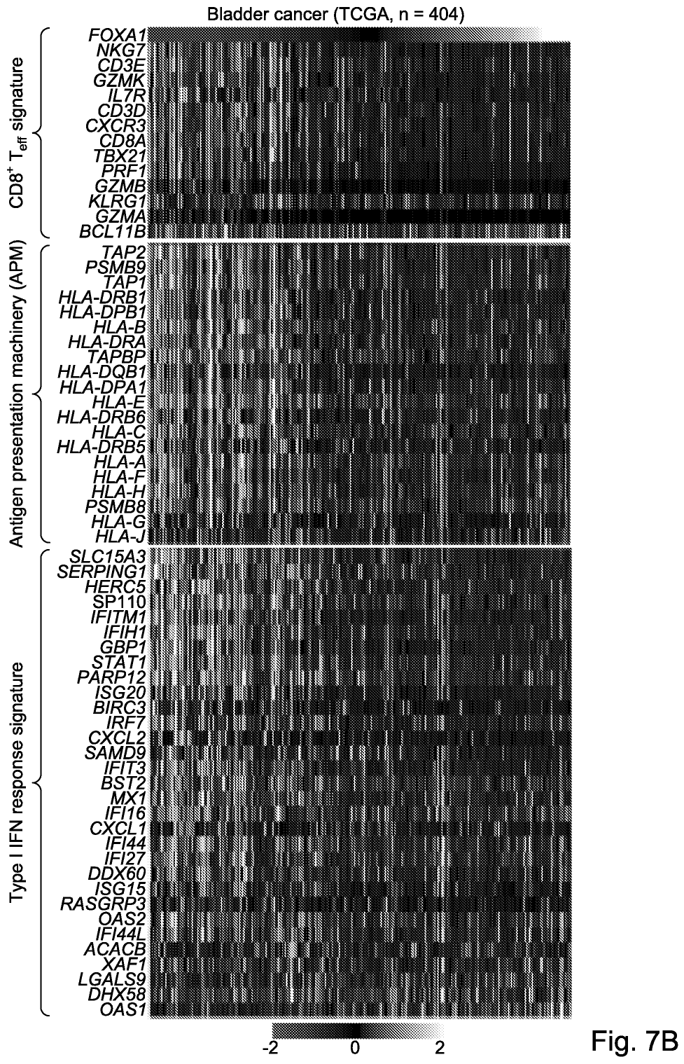

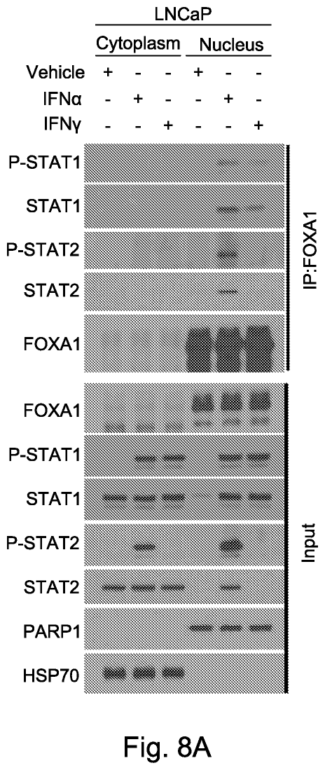

Activation of interferon (IFN) (including type I, II and III IFN) signaling in tumor is essential in CTL-mediated cancer cell killing. By utilizing the interferon stimulation response element-based luciferase reporter (ISRE-luc) for type I and III IFN and IFN-γ-activated sequence-based luciferase reporter (GAS-luc) for type II IFN as readouts, it was examined whether FOXA1 exerts any inhibitory effect on IFN signaling. It was found that ectopic expression of FOXA1 in 293T cells, which express little or no endogenous FOXA1, strongly inhibited type I/III IFN reporter gene activity in a dose-dependent manner and modestly suppressed type II IFN reporter activity at a high dose (FIG. 5, A and B). To corroborate FOXA1 regulation of IFN signaling in clinical specimens, the correlation between the expression of FOXA1 and IFN response signature, which has been shown to be associated with favorable prognosis in melanoma, was evaluated in different datasets. It was demonstrated that FOXA1 expression significantly inversely correlated with IFN response signature in prostate, breast, and bladder cancer patient specimens (FIG. 5C, FIG. 6, and FIG. 7). Moreover, IFNα treatment induced robust expression of IFN-responsive genes only in cell lines with little or no expression of FOXA1, but not in FOXA1 well-expressed cell lines (FIG. 5, B and D). These data indicate that FOXA1 is a negative regulator of IFN signaling in different cancer types in culture and patients.

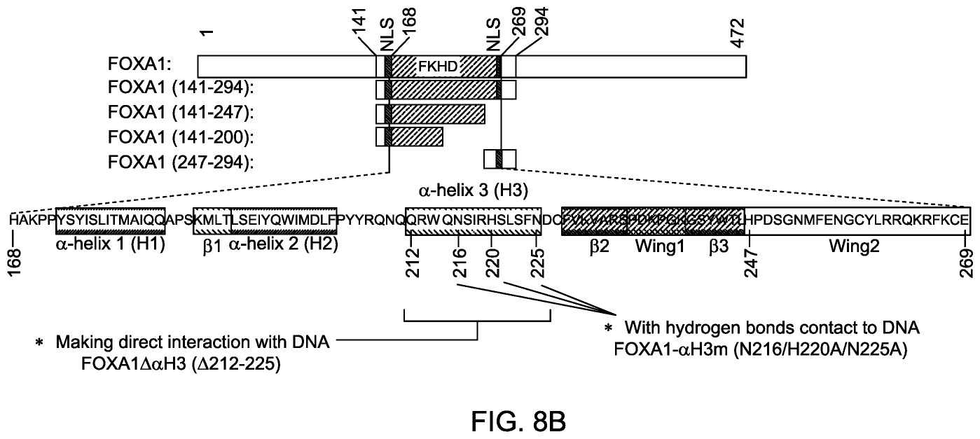

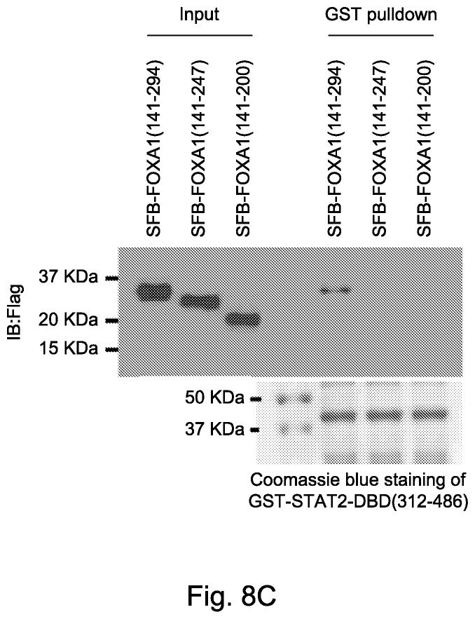

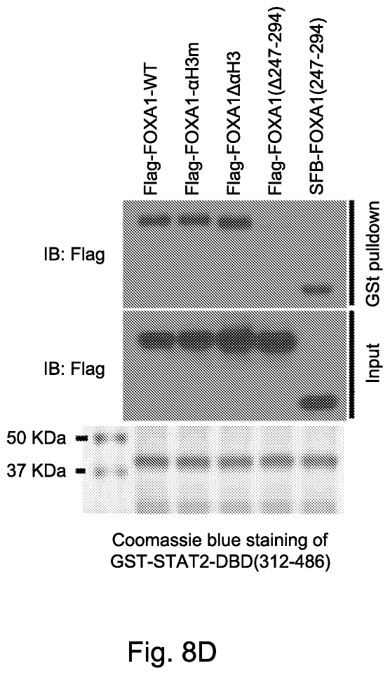

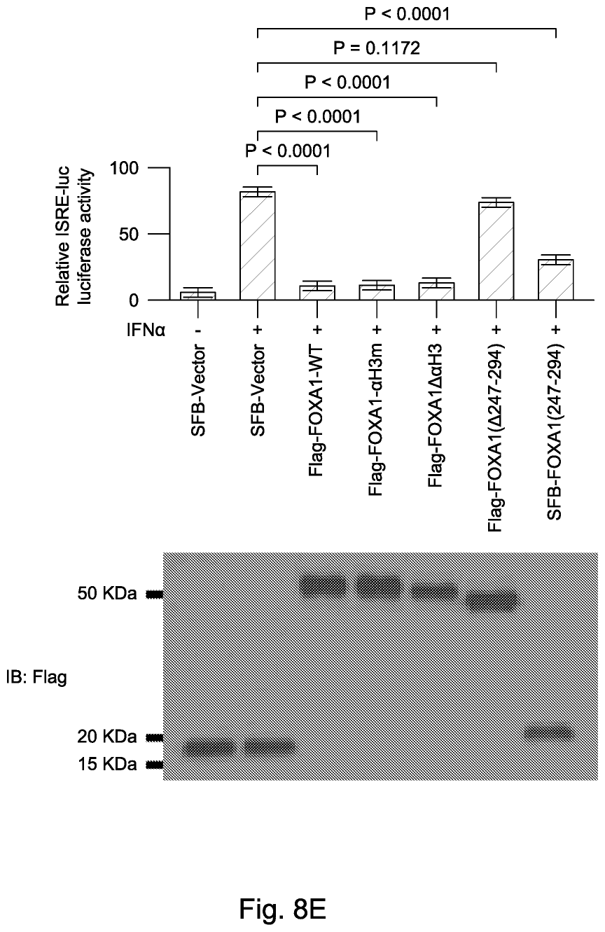

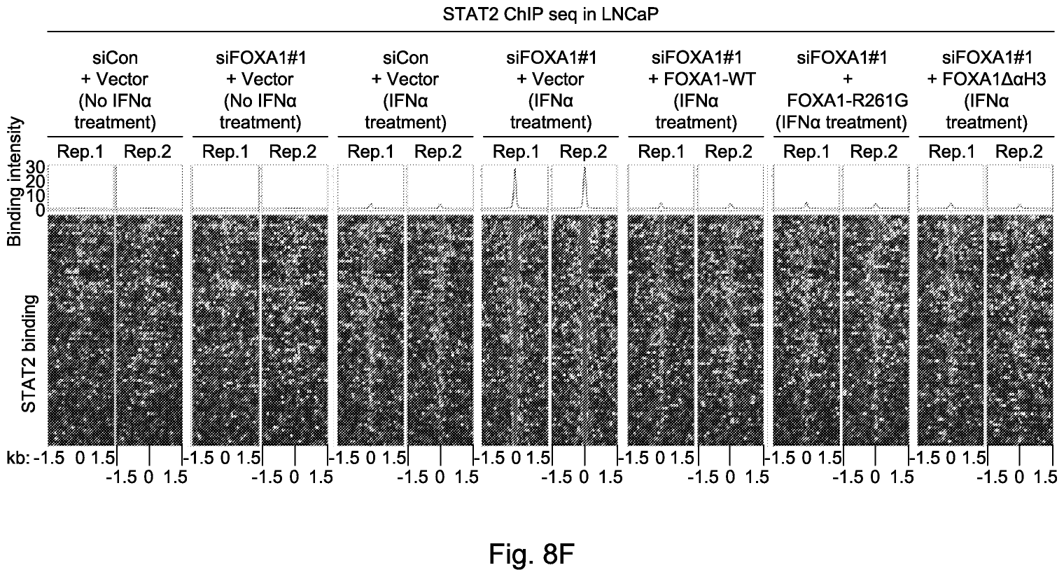

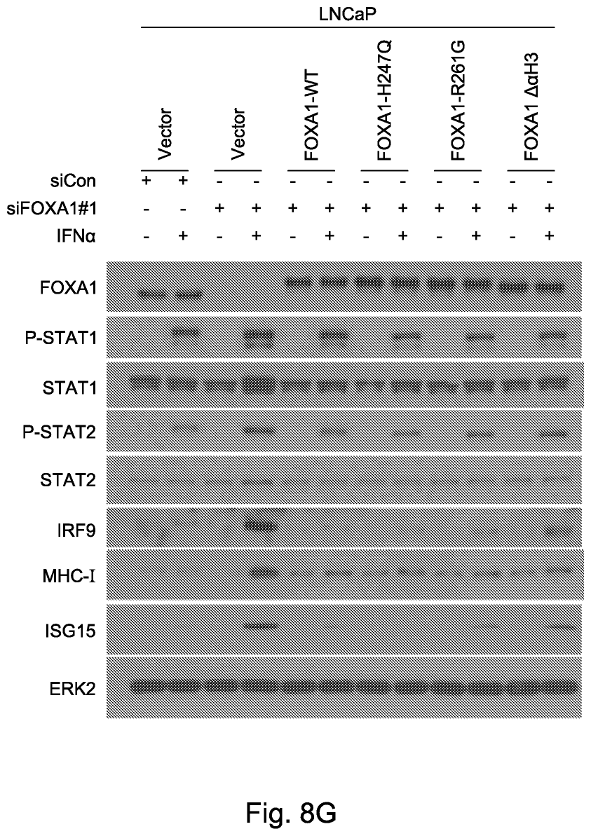

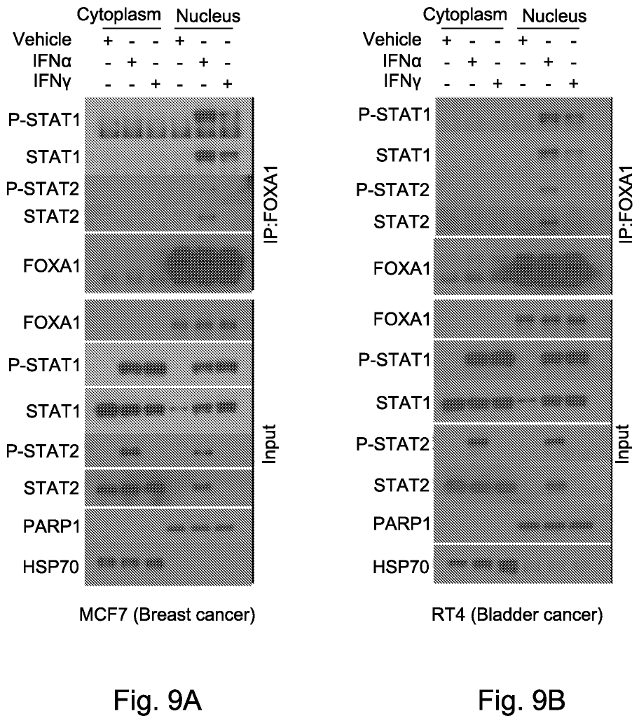

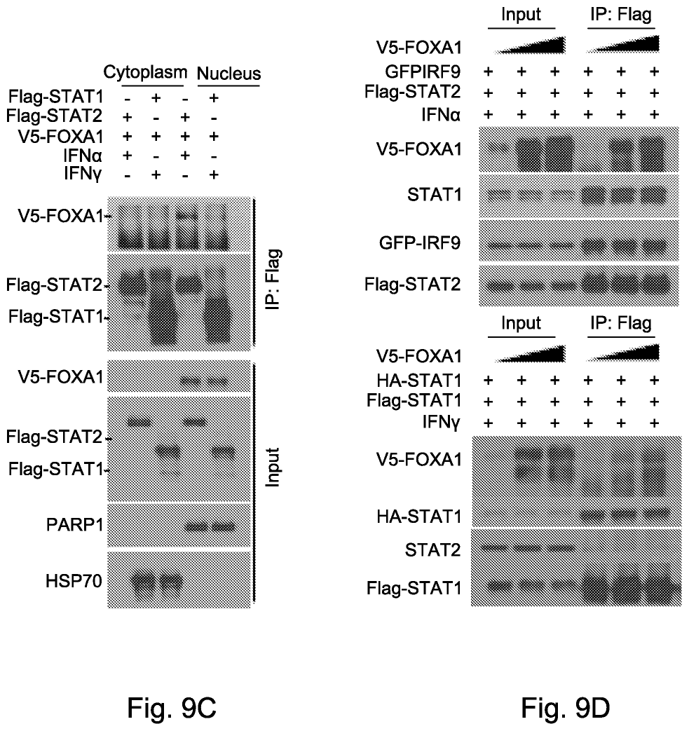

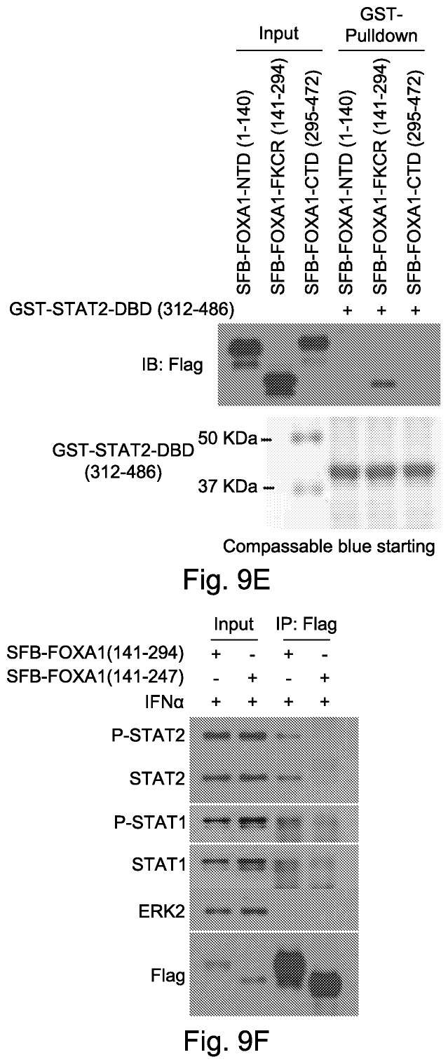

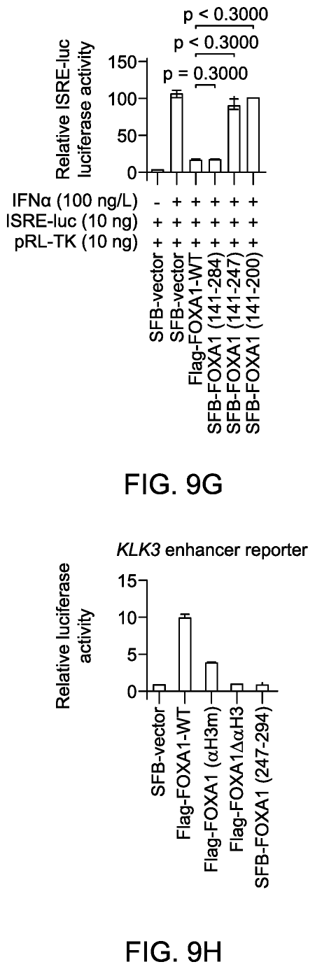

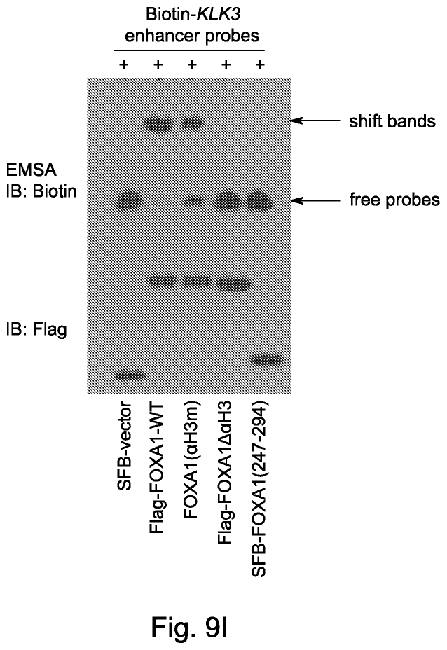

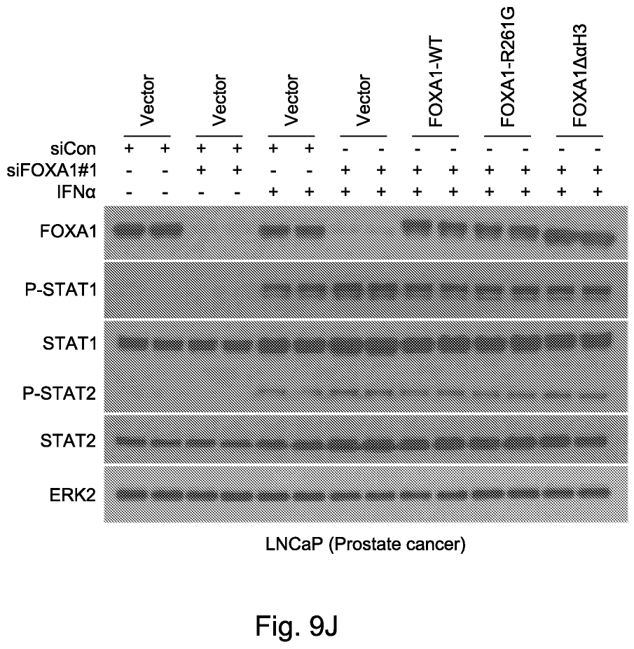

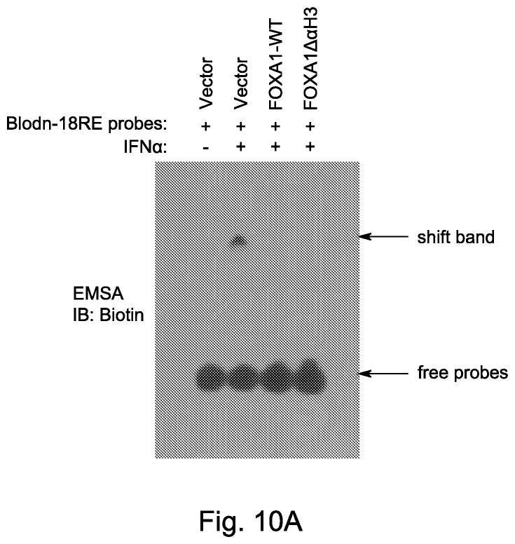

To explore the molecular mechanism underlying FOXA1 inhibition of IFN signaling, the effect of FOXA1 on phosphorylation of STAT1 and STAT2, two major effectors of type I/III and II IFNs, was examined. It was found that FOXA1 expression resulted in little or no changes in STAT1 and STAT2 phosphorylation in 293T cells (FIG. 5A). Co-immunoprecipitation (Co-IP) assay reveals that endogenous FOXA1 interacted with endogenous STAT1 and STAT2 upon IFNα stimulation and bound to STAT1 in response to IFNγ treatment at the nucleus of prostate, breast and bladder cancer cell lines (FIG. 8A, and FIG. 9, A and B). Consistent with the finding that the inhibitory effect of FOXA1 on type I/III IFN activity (mediated mainly by STAT1/STAT2 heterodimer) was much greater than the type II IFN activity (mediated mainly by STAT1 homodimer), FOXA1 association with the STAT1/STAT2 heterodimer was stronger than its binding with the STAT1 homodimer (FIG. 8A, and FIG. 9, A and B). In support of these observations, co-IP assay showed that STAT2 bound more FOXA1 protein than STAT1 in the nucleus (FIG. 9C). Dimerization of STAT1/STAT2 proteins is important for them to bind DNA and promote gene transcription upon IFN stimulation. It was further determined whether FOXA1 overexpression impairs STAT dimer formation. Increased expression of FOXA1 had no effect on the formation of STAT1-STAT2-IRF9 and STAT1-STAT1 complexes in 293T cells treated with IFNα and IFNγ respectively (FIG. 9D).

It was next sought to determine which region in STAT and FOXA1 mediates their interaction. Mutagenesis and glutathione S-transferase (GST) pull down assays showed that a forkhead domain (FKHD, a.a. 168-269)-containing region (FKCR, a.a. 141-294) in FOXA1 binds to the STAT2 DNA binding domain (a.a. 312-486) (FIG. 9E). To further narrow down the STAT-inhibitory region in FOXA1, two additional C-terminal truncation mutants of FKCR was constructed (FIG. 8B). GST pull down and co-IP assays showed that deletion of the Wing2 (a.a. 247-269)-containing region (a.a. 247-294, termed W2PLUS region) abolished FOXA1 interaction with STAT2 DBD in vitro and in cells (FIG. 8, B and C, and FIG. 9F). The protein binding result is further supported by the finding in the luciferase reporter gene assay (FIG. 9G). These data suggest that the W2PLUS fragment is important for FOXA1 suppression of IFN activity.

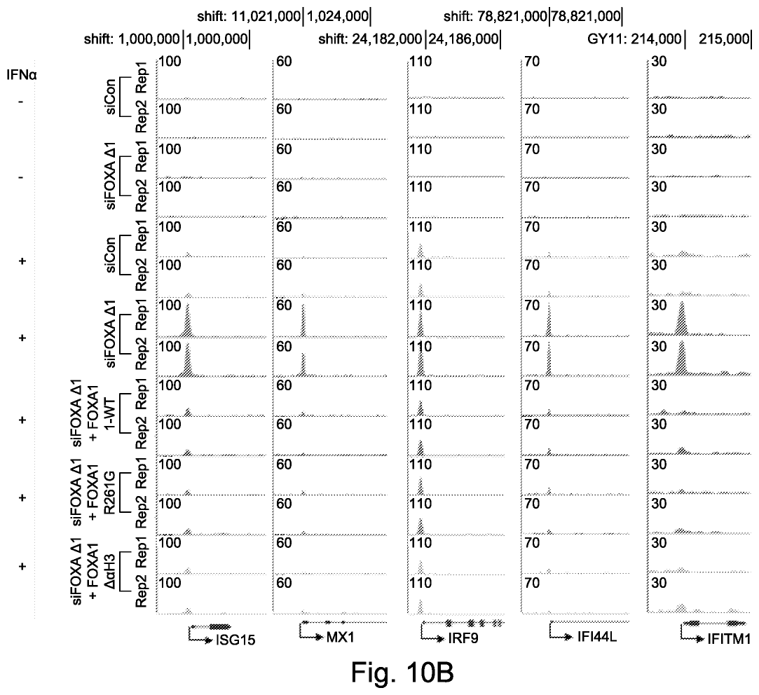

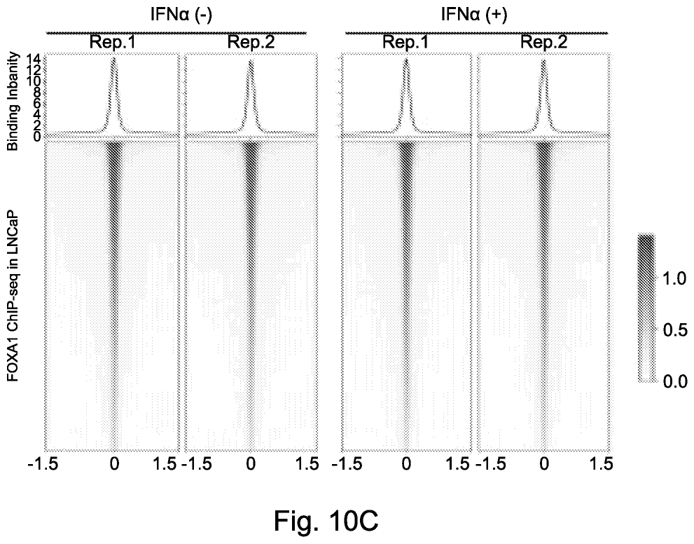

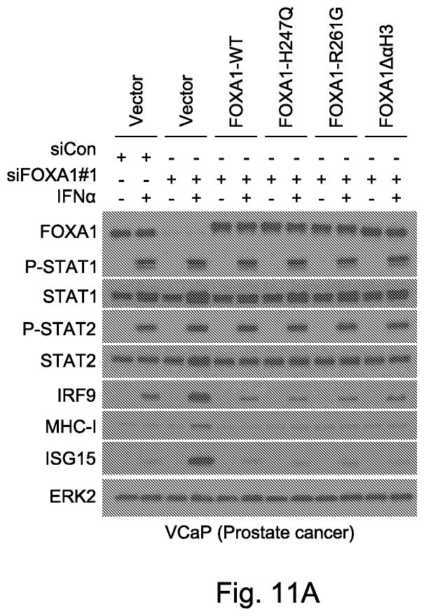

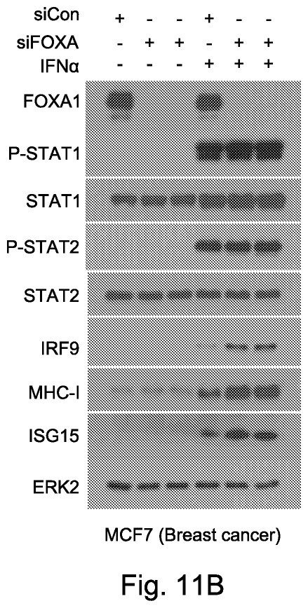

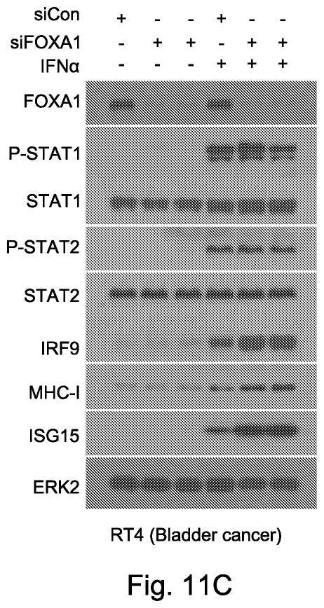

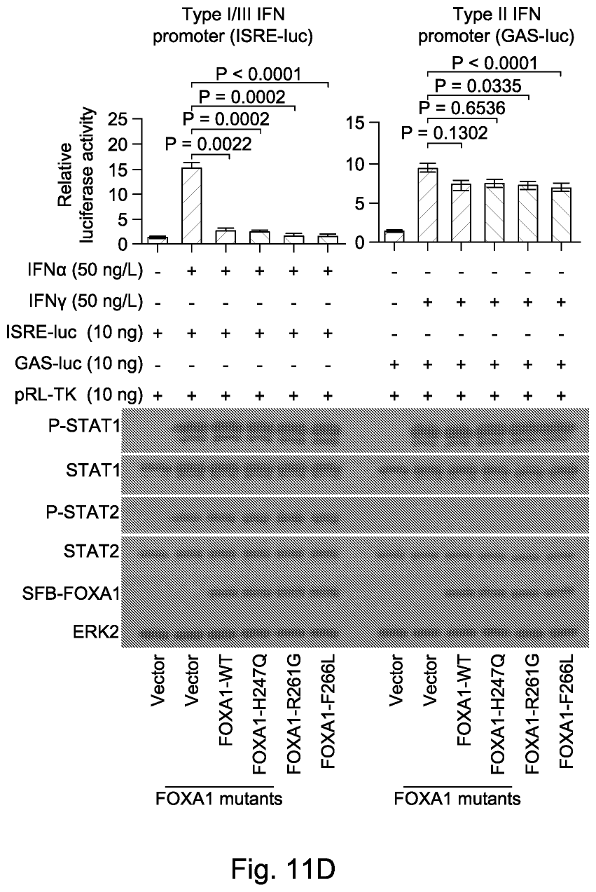

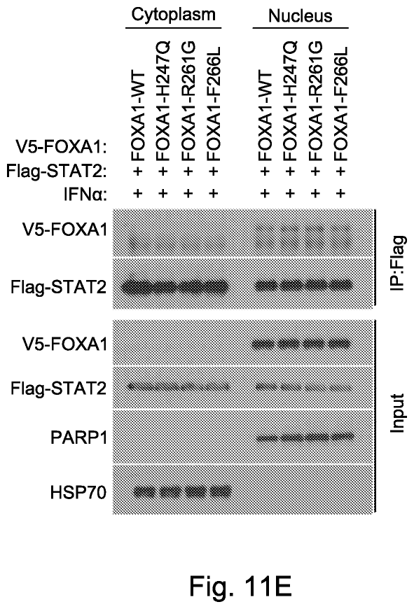

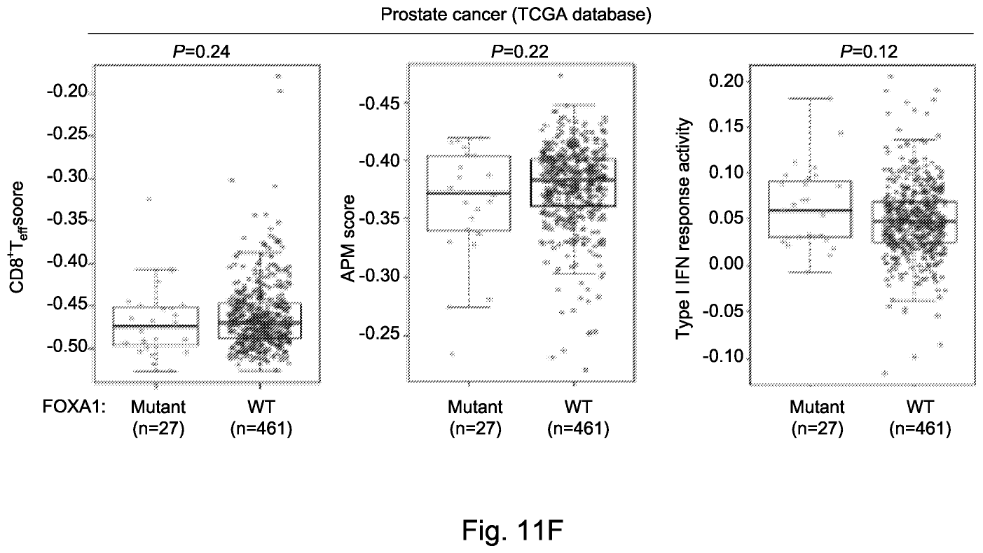

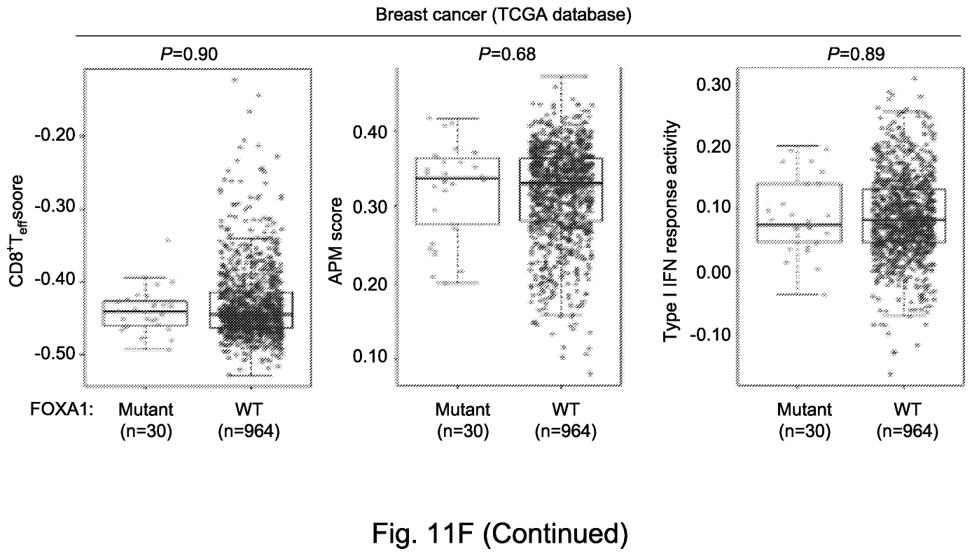

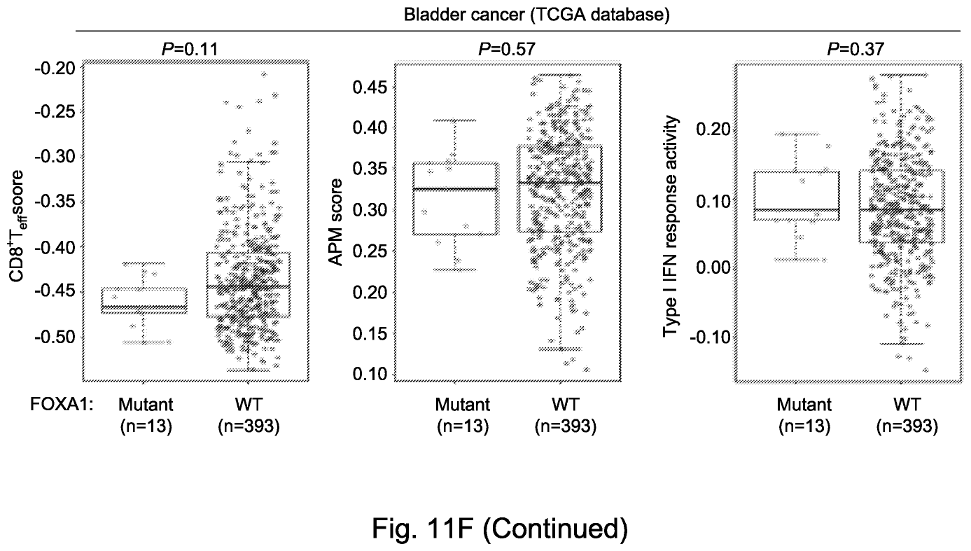

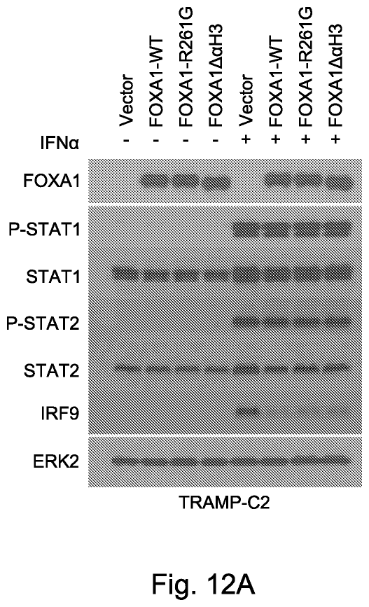

Next, it was sought to determine whether DNA binding ability of FOXA1 is essential to inhibit IFN signaling. The α-helix 3 (aH3, a.a. 212-225), especially residues N216, H220 and N225 in the FKHD domain of FOXA1 have direct contact with DNA. Expression vectors for FOXA1-N216A/H220A/N225A (FOXA1-aH3m) and FOXA1A212-225 (FOXA1ΔαH3), two DNA binding-deficient mutants in FOXA1 α-helix 3, were generated. The inability of these mutants to bind to the cognate FOXA1-targeting DNA sequence was confirmed using different methods (FIG. 9, H and I). These two mutants were still able to bind to STAT2 DBD (FIG. 8D) and inhibit IFN activity (FIG. 8, B and E), indicating that FOXA1 suppresses IFN signaling independently of its DNA binding function. Electrophoretic mobility shift assay (EMSA) showed that expression of both FOXA1-WT and FOXA1ΔαH3 inhibited IFNa-induced formation of DNA (ISRE)/protein complexes (FIG. 10A). Furthermore, the endogenous FOXA1 in LNCaP cells was knocked down using a small interfering RNA (siRNA) specifically targeting 3′ untranslated region (3′UTR) and rescued with expression of siRNA non- targetable WT FOXA1 and DNA binding-deficient mutant FOXA1ΔαH3 (FIG. 9J), and these stable cell lines were utilized for chromatin-immunoprecipitation-sequencing (ChIP-seq) using an STAT2 antibody. ChIP-seq data manifested that genome-wide DNA binding of STAT2 was substantially elevated upon IFNα stimulation in LNCaP cells and this effect was drastically enhanced by knockdown of endogenous FOXA1 (FIG. 8F and FIG. 10B). Restored expression of FOXA1 WT and FOXA1ΔαH3 invariably abolished the robust DNA binding of STAT2 detected in FOXA1-deficient cells (FIG. 8F and FIG. 10B), supporting the notion that FOXA1 suppression of STAT2 DNA binding and IFN signaling is independent of its DNA binding function. Notably, IFNα treatment had little or no effect on genome-wide chromatin engagement of FOXA1 in LNCaP cells (FIG. 10C). It was also confirmed that FOXA1 knockdown magnified the expression of type I IFN target genes at protein level, but this effect was reversed by re-expression of FOXA1-WT and FOXA1ΔαH3 in both LNCaP and VCaP cells (FIG. 8G and FIG. 11A). These data indicated that the FOXA1 expression level is critical in constraining IFNα response in prostate cancer cells, and similar results were observed in breast and bladder cancer cells (FIG. 11, B and C).

The role of cancer-associated FOXA1 mutants in regulating IFN activity was investigated. FOXA1 prostate cancer-derived ‘hotspot’ mutants, including FOXA1-H247Q, FOXA1-R261G, and FOXA1-F266L, bound to and inhibited IFN reporter gene activities to an extent similar to the WT counterpart (FIG. 11, D and E). Moreover, ChIP-seq analysis revealed that similar to WT FOXA1, restored expression of R261G mutant completely reversed FOXA1 depletion-enhanced genome-wide DNA binding of STAT2 in IFNα-treated LNCaP cells (FIG. 8F, FIG. 9J, and FIG. 10B). These findings were further confirmed by western blot analysis of IFN responsive genes such as MHC-I and ISG15, and similar results were obtained in both LNCaP and VCaP prostate cancer cell lines expressing another FOXA1 mutant H247Q (FIG. 8G and FIG. 11A). In support of these observations in cell lines, there was no significant difference in expression of type I IFN response genes, CD8+ T cell effector genes, and APM genes between FOXA1 mutated and WT prostate, breast, and bladder cancers of TCGA cohorts (FIG. 11F). These data suggest that prostate cancer-derived FOXA1 mutations can inhibit IFN activities, APM gene expression, and cancer immunity to an extent similar to the WT FOXA1.

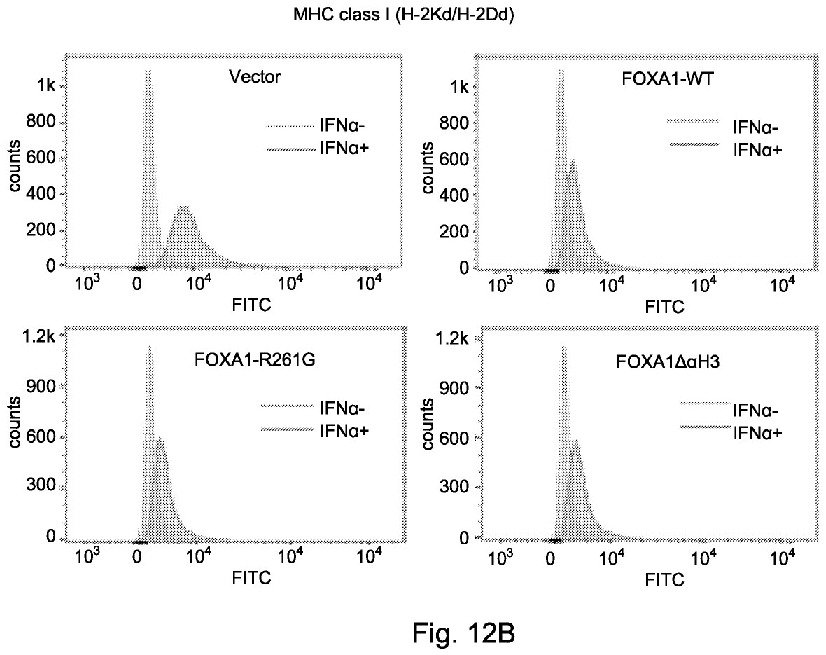

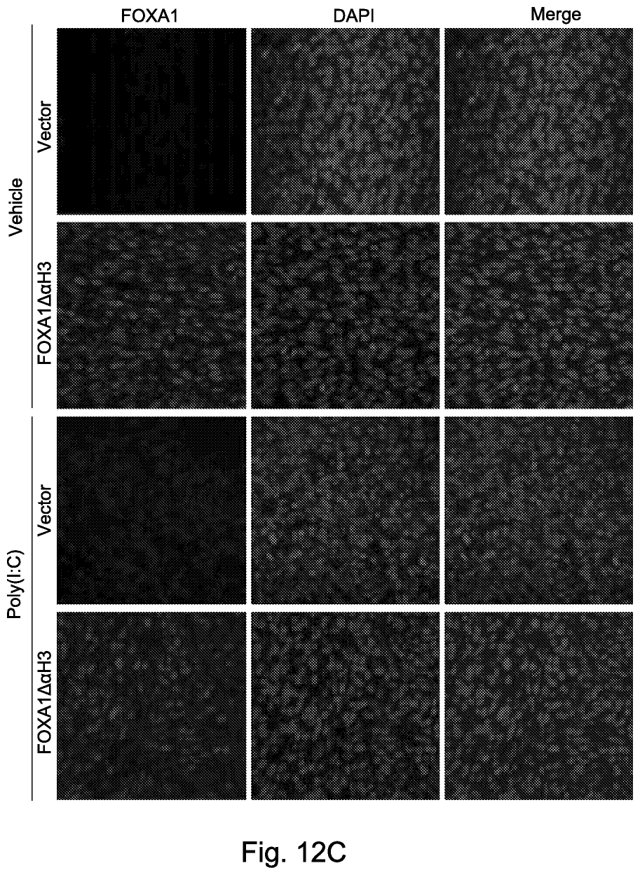

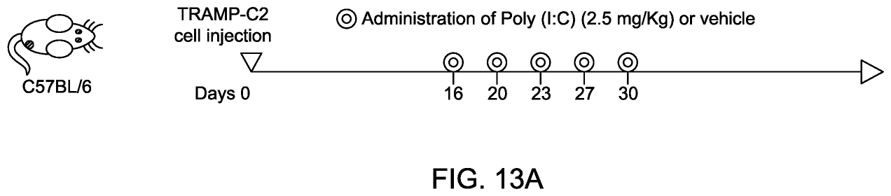

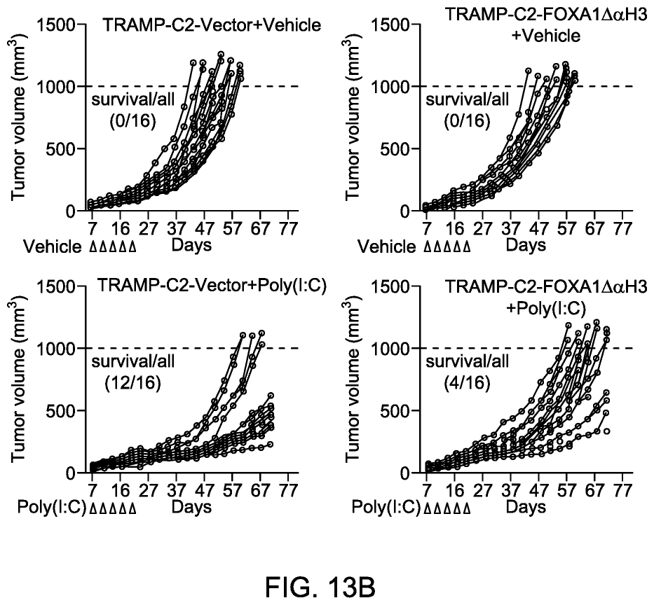

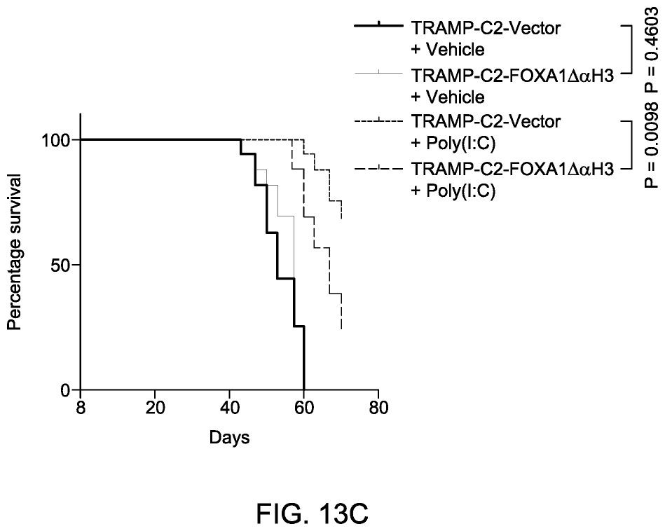

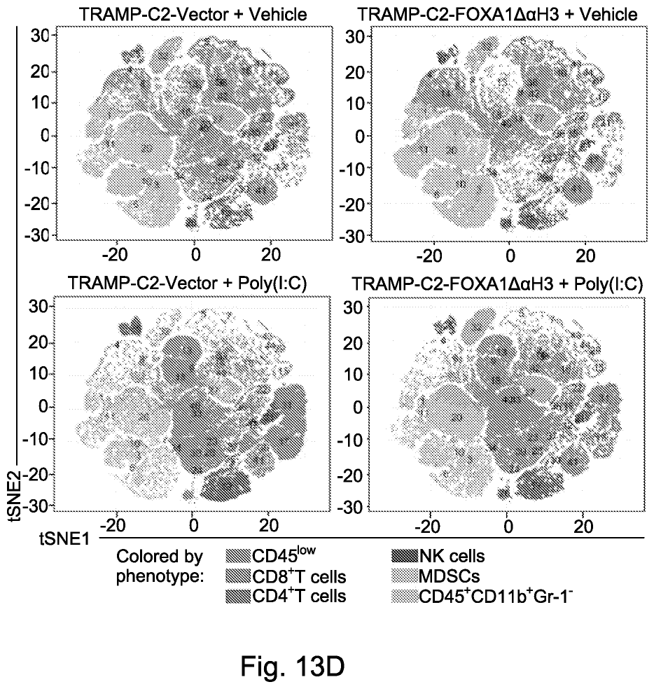

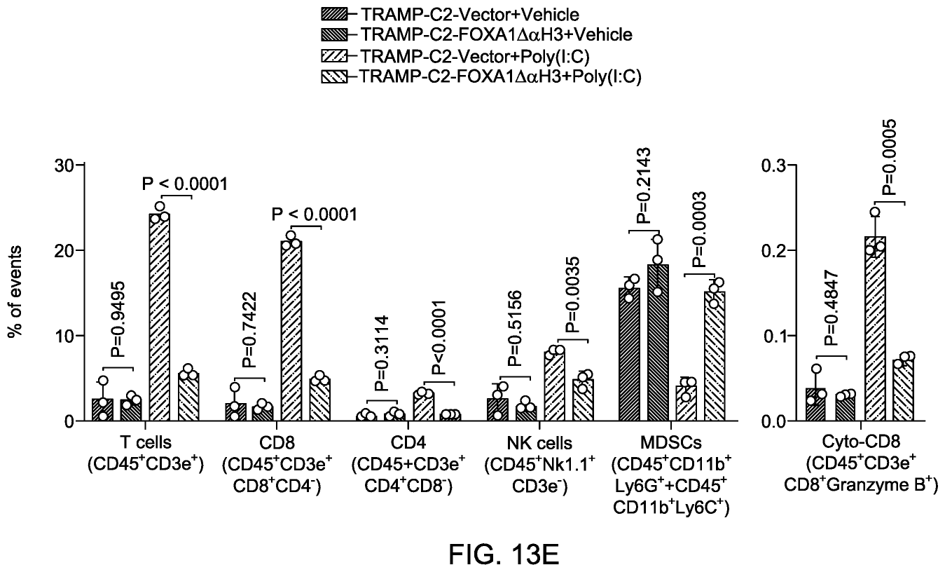

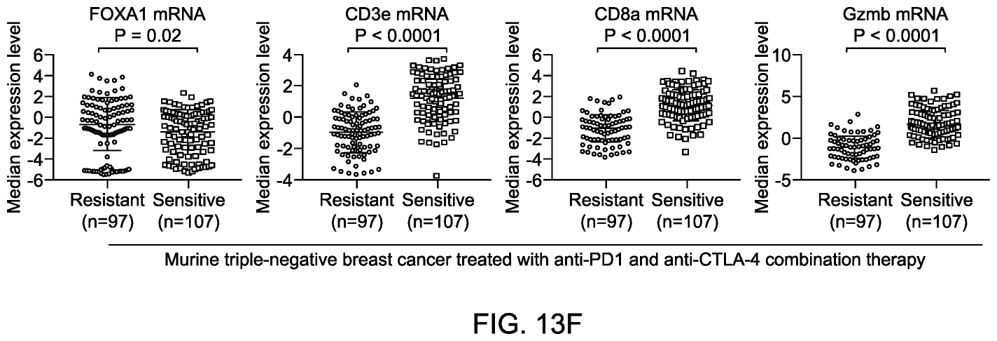

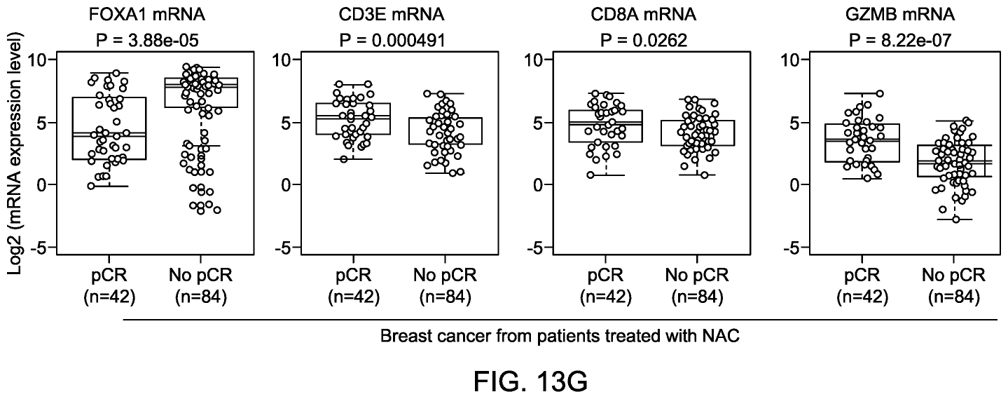

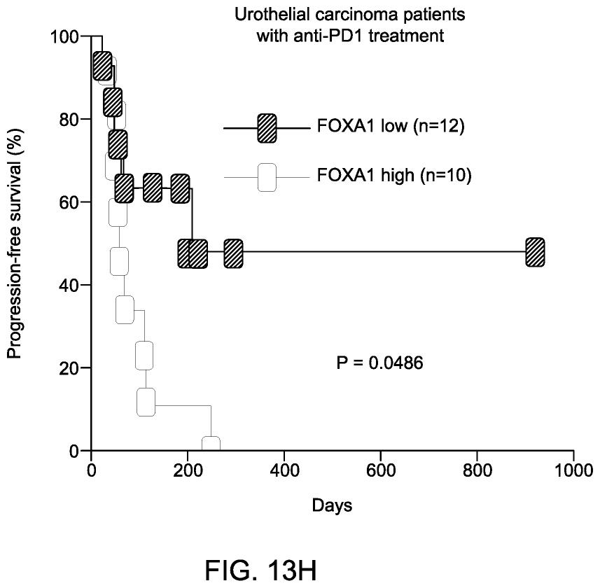

To directly explore the role of FOXA1 in inhibiting cancer immune response, stable murine prostate cancer TRAMP-C2 cell lines overexpressing FOXA1-WT, cancer-associated mutant FOXA1-R261G and DNA binding-deficient mutant FOXA1ΔαH3, were established. Similar to the results in human prostate cancer cells (FIG. 9G), overexpression of FOXA1-WT, FOXA1-R261G and FOXA1ΔαH3 equivalently inhibited IFNα signaling in TRAMP-C2 cells (FIG. 12, A and B). Next, TRAMP-C2-Vector and TRAMP-C2-FOXA1ΔαH3 cells were injected into syngeneic C57BL/6 mice and intratumorally injected Poly(I:C) to trigger type I IFN immune response (FIG. 13A). Poly(I:C) administration decreased the growth of control (TRAMP-C2-Vector) tumors in the majority of mice and prolonged the overall mouse survival (FIG. 13, B and C). On the contrary, the tumor growth-inhibitory effect of Poly(I:C) was largely diminished in TRAMP-C2-FOXA1 Δ αH3 tumors (FIG. 13, B and C, and FIG. 12C). Furthermore, TILs, especially CD8⁺ T and NK cells, were discernibly increased in TRAMP-C2-Vector tumors treated with Poly(I:C), but such effect was diminished in TRAMP-C2-FOXA1ΔαH3 tumors (FIG. 13, D and E, and FIG. 12, C and D). CD11b⁺Gr1⁺myeloid-derived suppressor cells (MDSCs) which play important roles in T cell suppression were reduced upon Poly(I:C) stimulation in TRAMP-C2-Vector tumors but not in TRAMP-C2-FOXA1ΔαH3 tumors (FIG. 13, D and E). These data suggest that FOXA1 suppresses cancer immunity in vivo.

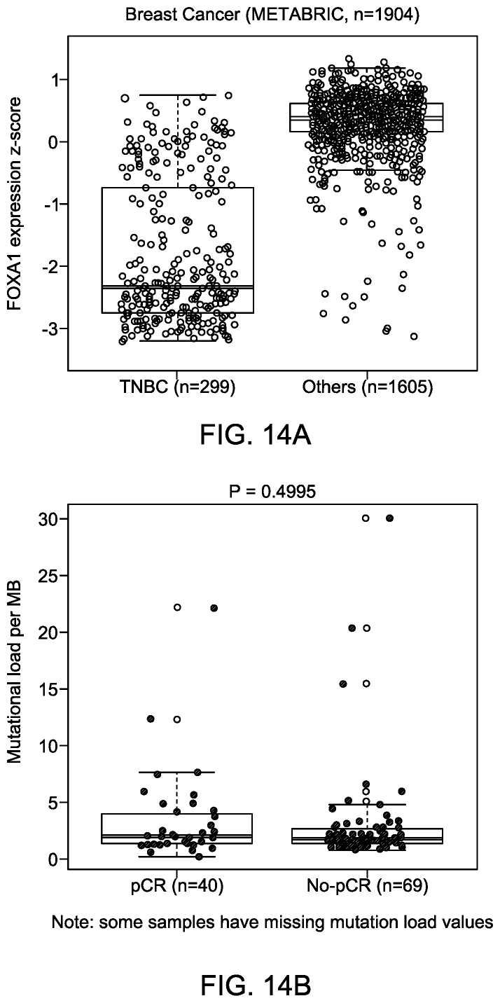

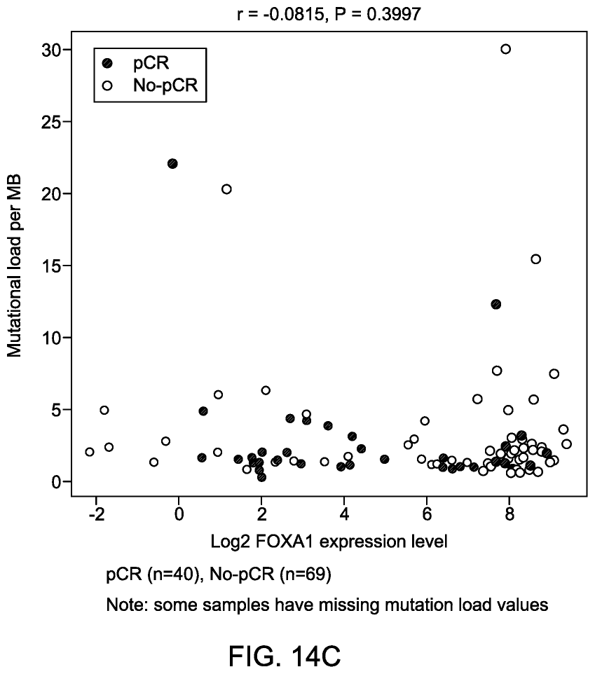

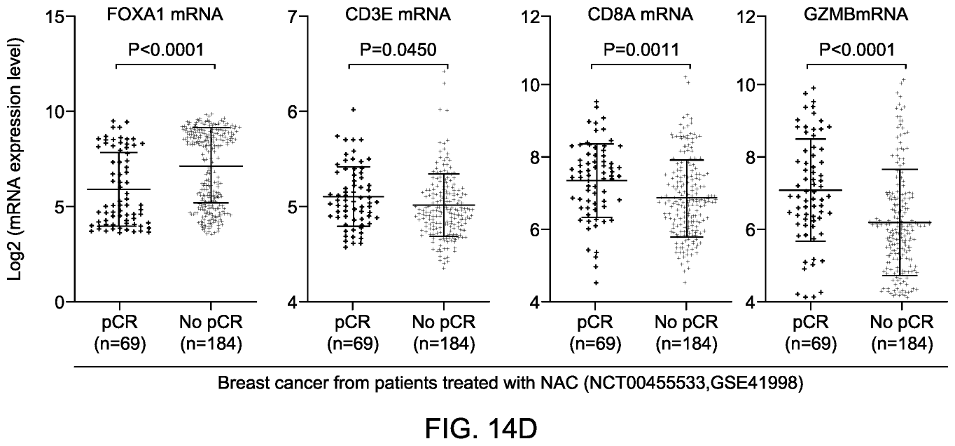

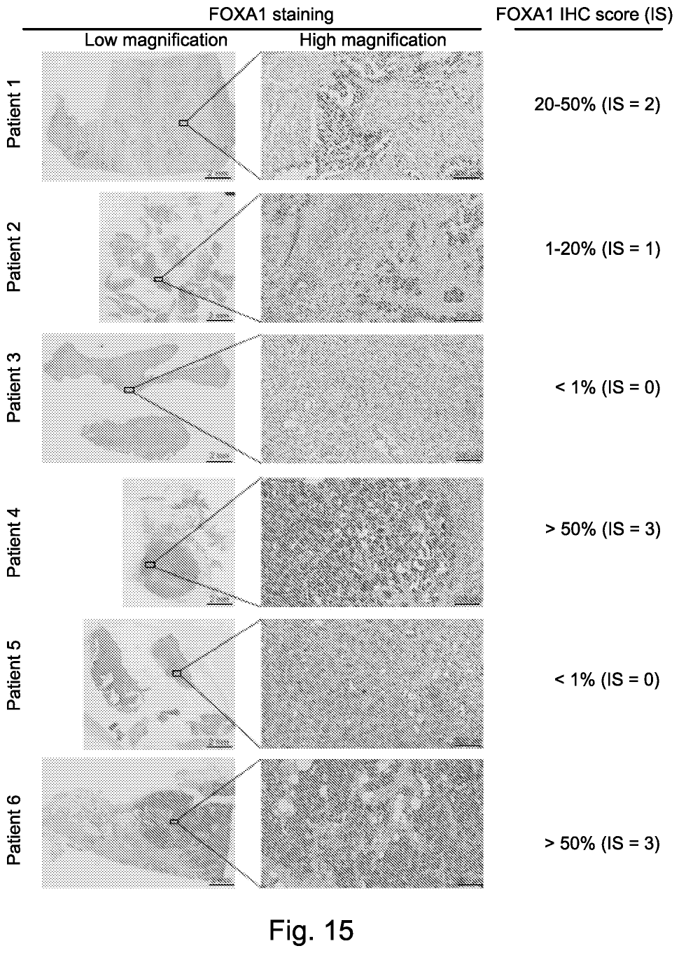

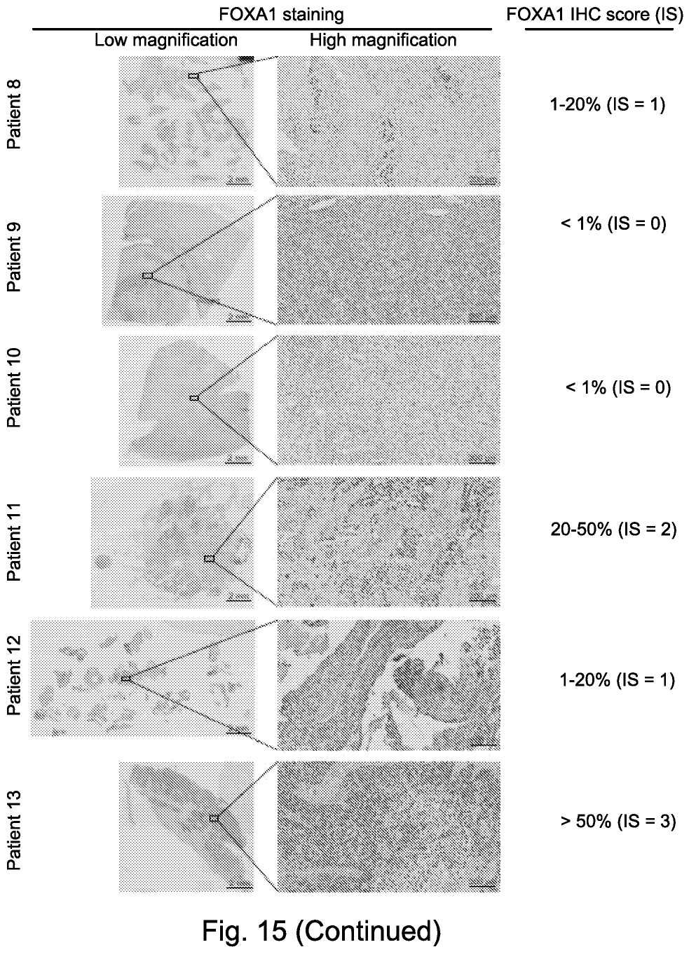

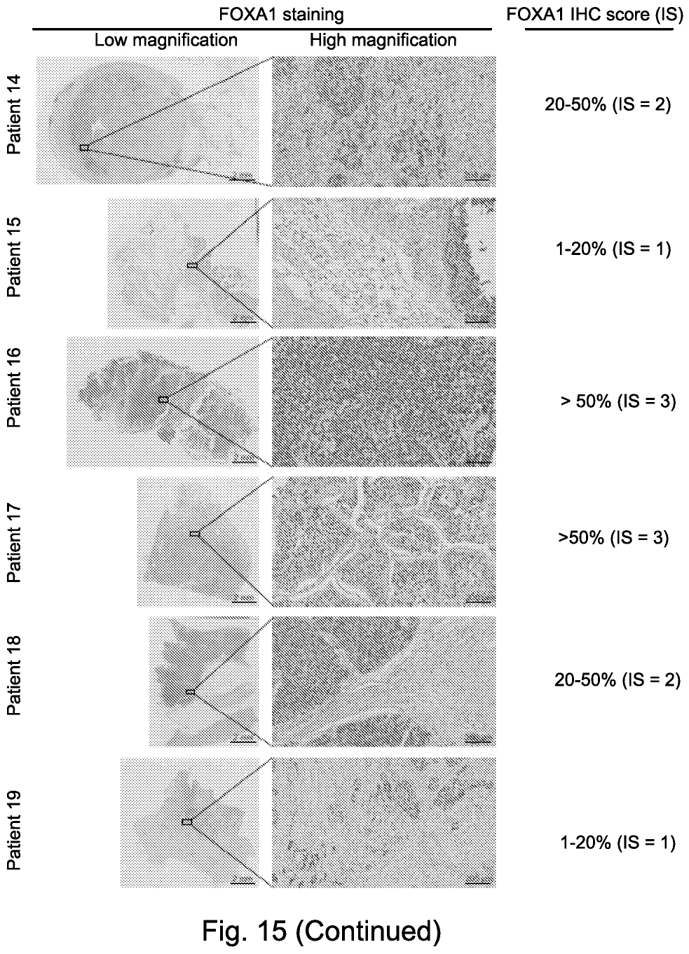

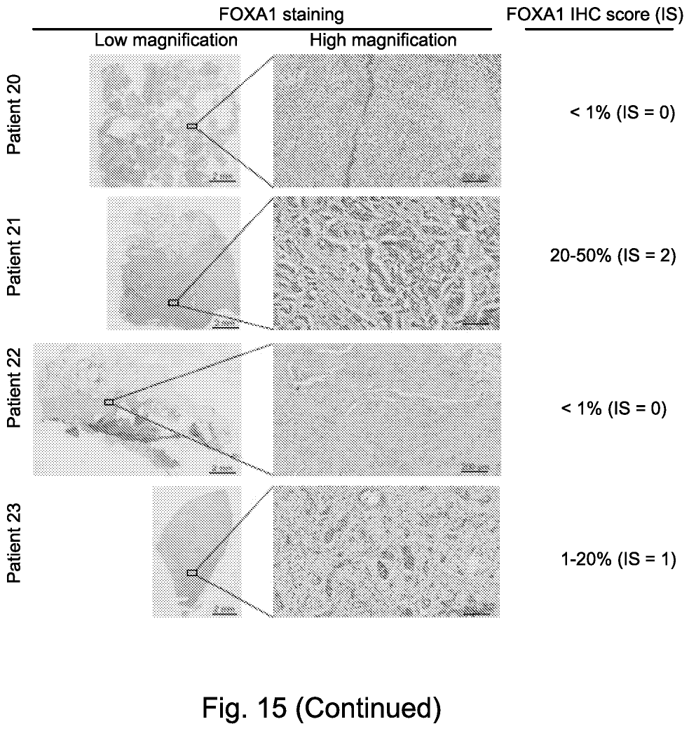

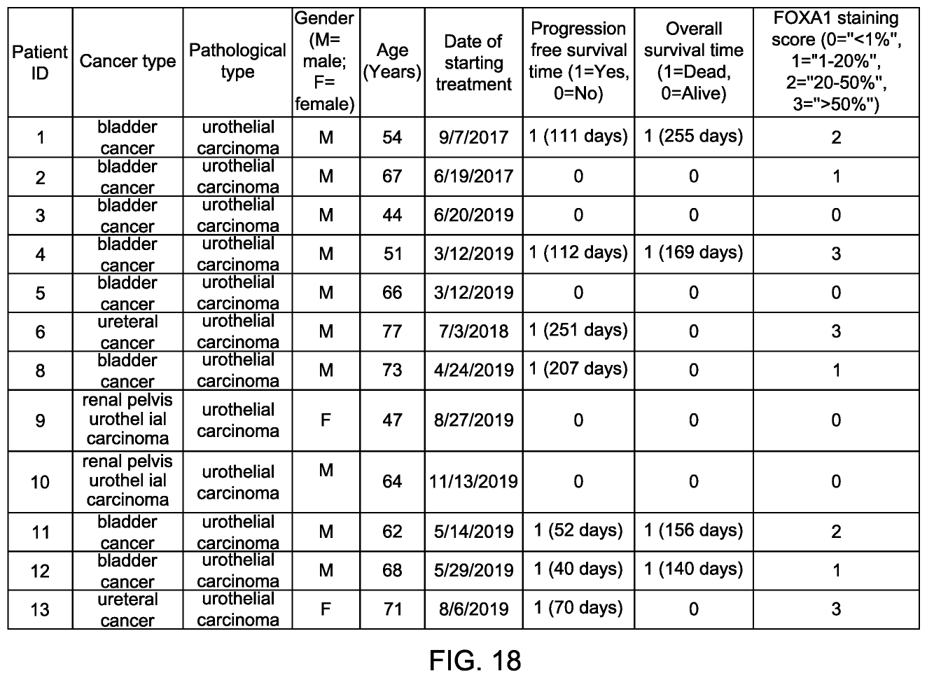

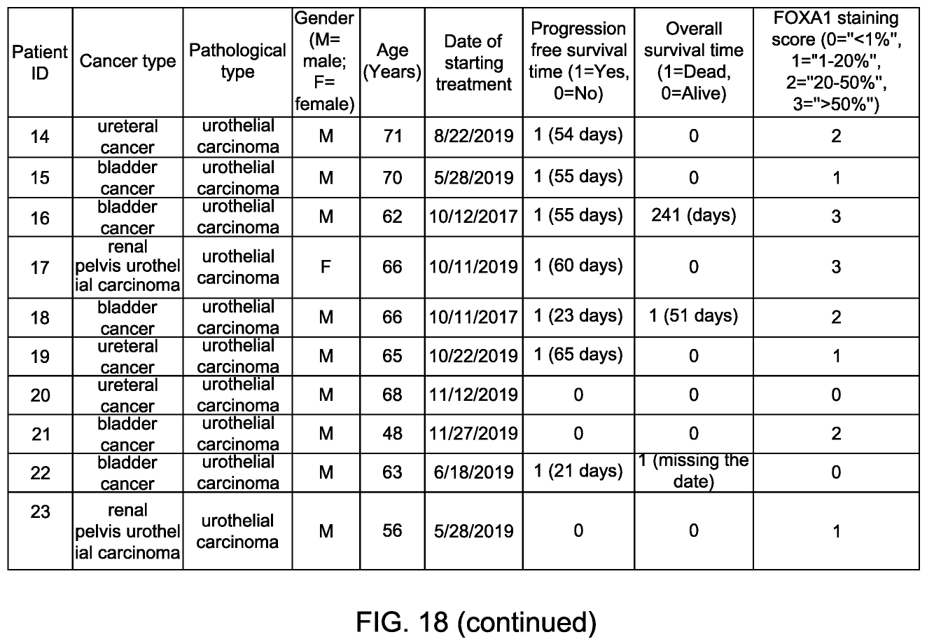

To validate the findings from the TRAMP-C2 mouse prostate cancer model, the correlation between Foxa1 expression and anti-PD1 and anti-CTLA-4 therapy response was further examined in a cohort of 204 murine triple-negative breast cancers (TNBCs). RNA-seq data analysis showed that increased Foxal expression significantly associated with tumor resistance to ICI therapy in mice (FIG. 13F). In contrast, higher level expression of the effect T cell markers such as CD3e, CD8a and Gzmb strongly correlated with tumor response to ICI therapy in these tumors (FIG. 13F). While FOXA1 expression in TNBCs was generally lower than that in non-TNBC tumors in the METABRIC cohort (Cancer Genome Atlas, Nature 490:61-70 (2012); and Pereira et al., Nat Commun 7:11479 (2016)), a subset of TNBC tumors did express FOXA1 at levels comparable to those in non-TNBC tumors (FIG. 14A). RNA-seq data from the cohort of breast cancer patients treated with neoadjuvant chemotherapy (NAC) was also analyzed and it was demonstrated that FOXA1 levels were significantly higher in tumors without pCR than those with pCR whereas expression of the effect T cell markers such as CD3E, CD8A and GZMB was positively correlated with pCR (FIG. 13G), and these effects appear to be independent of overall mutation burden in these tumors (FIG. 14, B and C). This observation is consistent with the result from the meta-analysis of microarray data in a breast cancer cohort (FIG. 14D). Furthermore, FOXA1 protein expression was examined by immunohistochemistry (IHC) in pre-treatment urothelial carcinoma specimen from twenty-three patients with anti-PD1 therapy. This analysis demonstrated that patients with higher FOXA1 protein levels had much lower rates of progression-free survival (FIG. 13H, FIG. 15, and Table 4 (FIG. 18)). Together, these data suggest that FOXA1 overexpression contributes to immune evasion and immune- and chemo-therapy resistance in breast and bladder cancer patients.

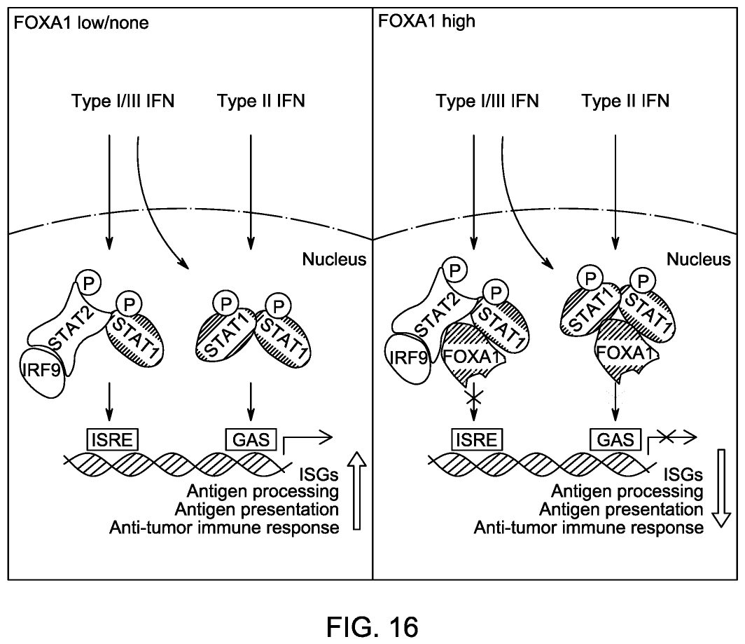

The findings in the present study demonstrate that FOXA1 plays an important role in promoting cancer progression by suppressing IFN signaling, APM gene expression, and cancer immunity in a manner independent of its DNA binding function (FIG. 16). The findings in cultured cell lines, mouse model and patient samples indicate that FOXA1 inhibition of IFN and APM and signaling is likely independent of FOXA1 mutation status. Indeed, it appears that the reported tumor-promoting functions of FOXA1 mutations are dependent on DNA binding activity of FOXA1 (Parolia et al., Nature 571:413-418 (2019)). In contrast, the role of FOXA1 in suppression of cancer immunity identified here is DNA binding-independent. Thus, the data reveal that in addition to gene mutations, FOXA1 can also drive cancer progression through overexpression (FIG. 16).

FOXA1 is known as a pioneer factor for steroid hormone receptors such as androgen receptor (AR) and estrogen receptor (ER) and its expression is often associated with luminal phenotype of prostate and breast cancer. This study in breast cancers from patients shows that high levels expression of FOXA1 associate not only with lower rates of tumor response to neoadjuvant chemotherapy, but also with the lower numbers of TILs. Therefore, these findings provide a mechanistic explanation for the clinical observation that basal-like TNBC (FOXA1 low or none) have much higher rates of pathologic complete response (pCR) than luminal androgen-receptor (LAR)-positive TNBC and luminal types of breast cancer (FOXA1 high). This study identifies a new vulnerability for aggressive breast cancer (e.g. TNBC) and prostate cancer (e.g. NEPC), majority of which express little or none FOXA1. These findings also suggest that targeting FOXA1 could be an option to improve the efficacy of therapeutics such as chemotherapy on FOXA1-high tumor types such as luminal types of prostate and breast cancer.

In support of the findings in cancer cells in culture and in mice, it was demonstrated that increased FOXA1 expression significantly associates with ICI-based immunotherapy resistance in both murine TNBC tumors and bladder cancers in patients. Thus, these results suggest that FOXA1 expression level can be a strong biomarker to predict tumor response to immunotherapy. Additionally, exploration of a druggable approach to deplete FOXA1 level could be a viable strategy to convert the FOXA1-positve ‘immune-cold’ tumors to ‘immune-hot’ tumors in clinic.

### Materials and Methods

### Computational Analysis

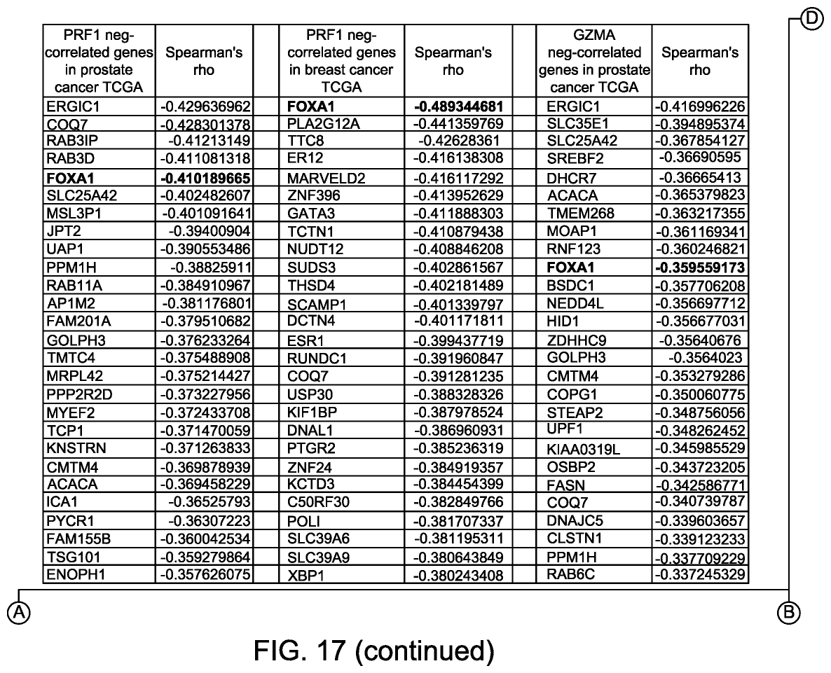

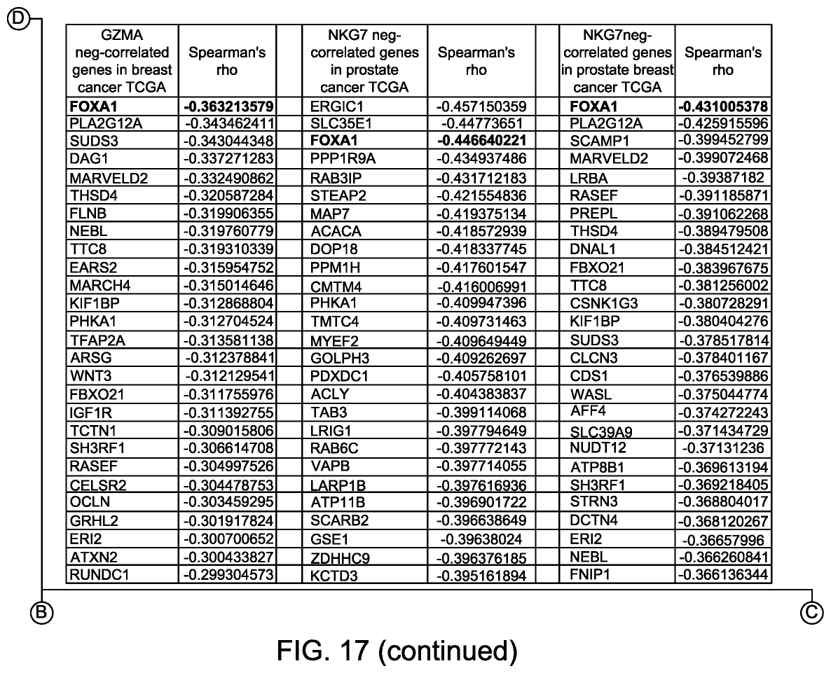

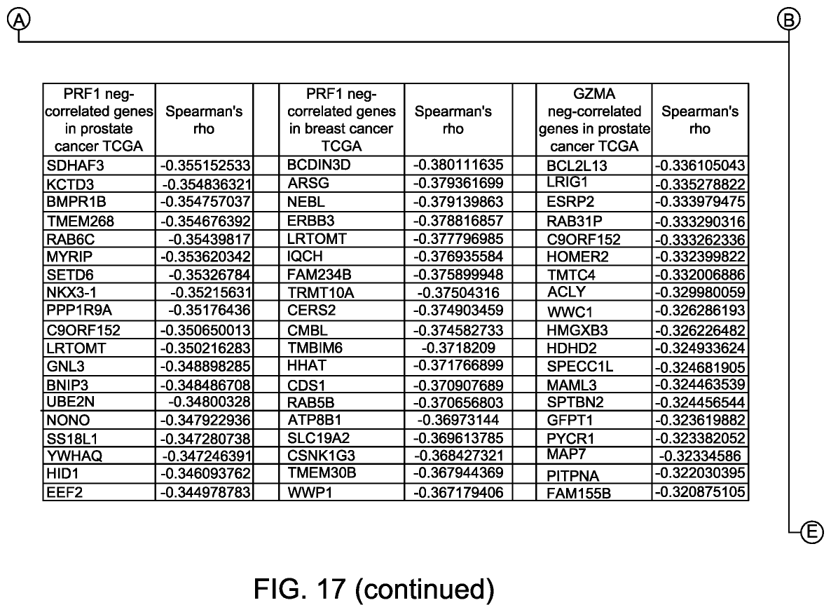

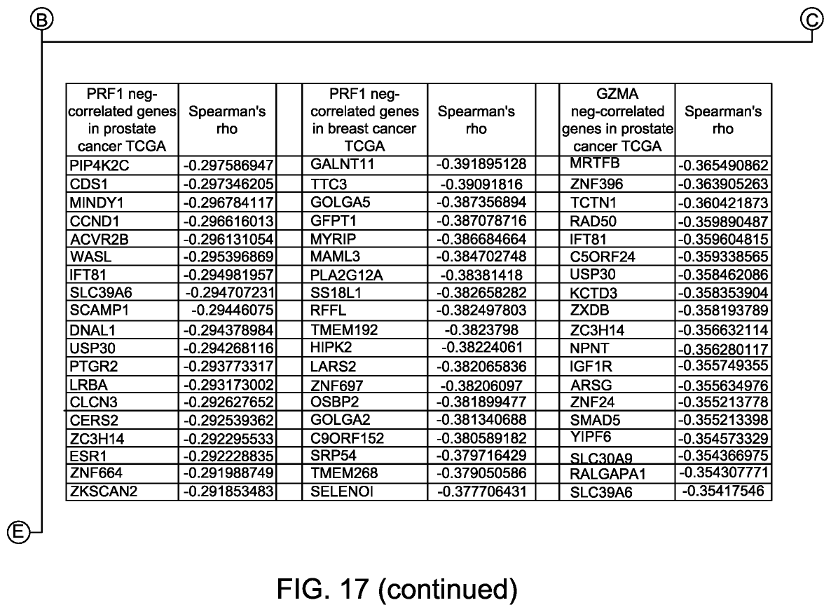

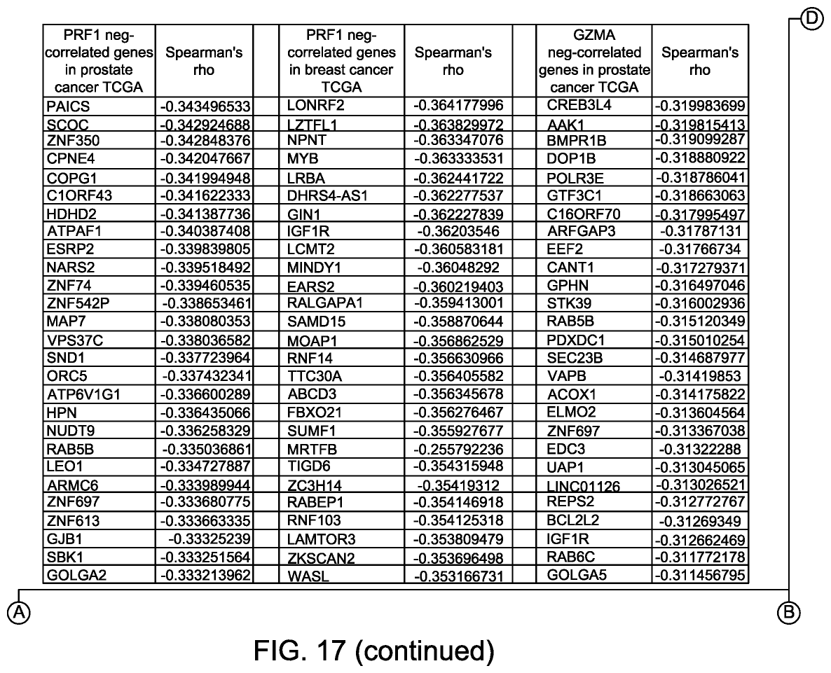

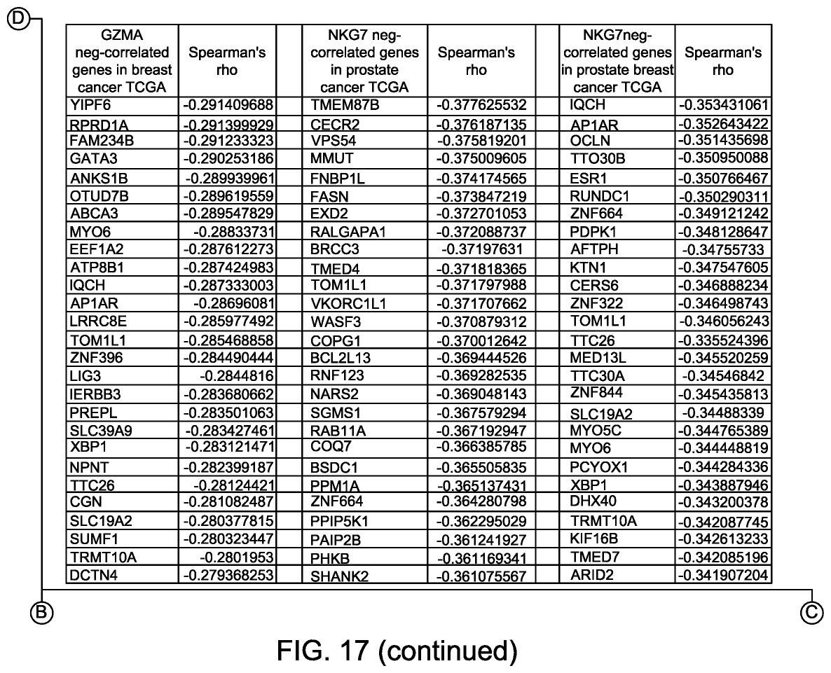

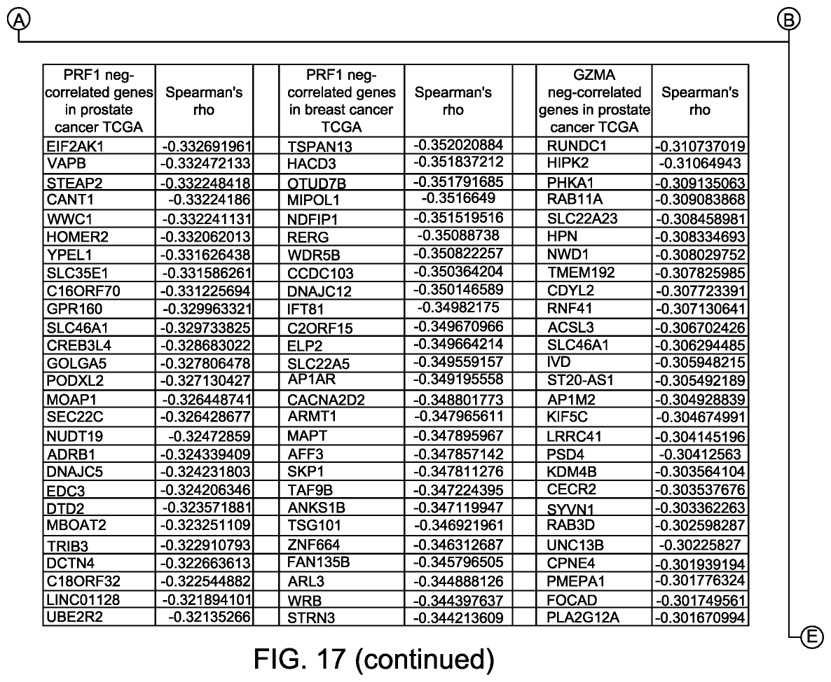

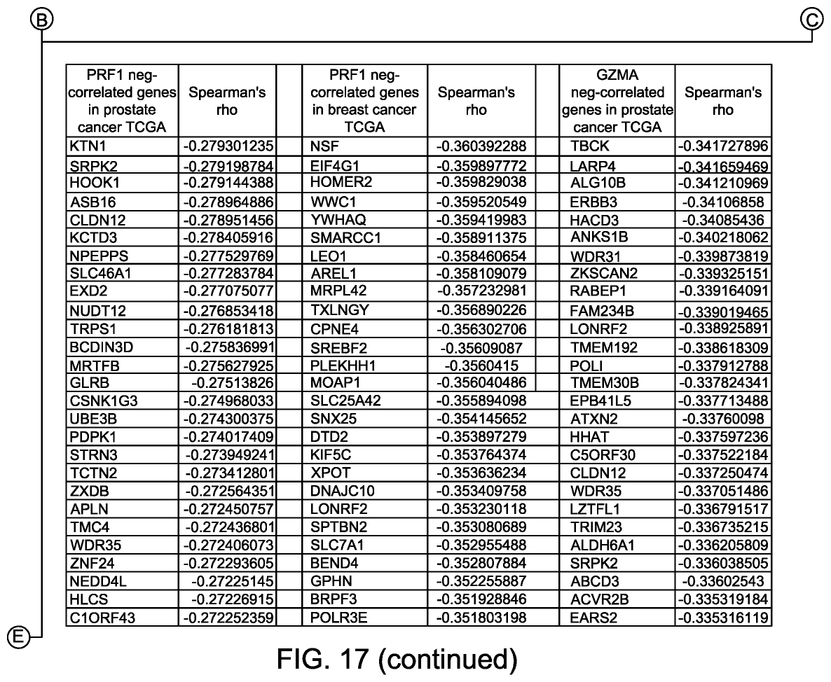

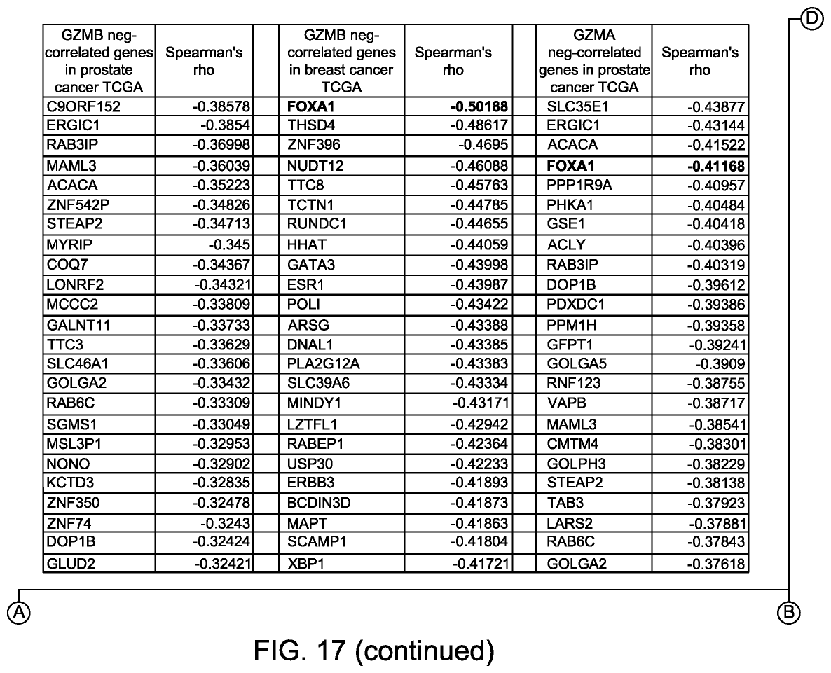

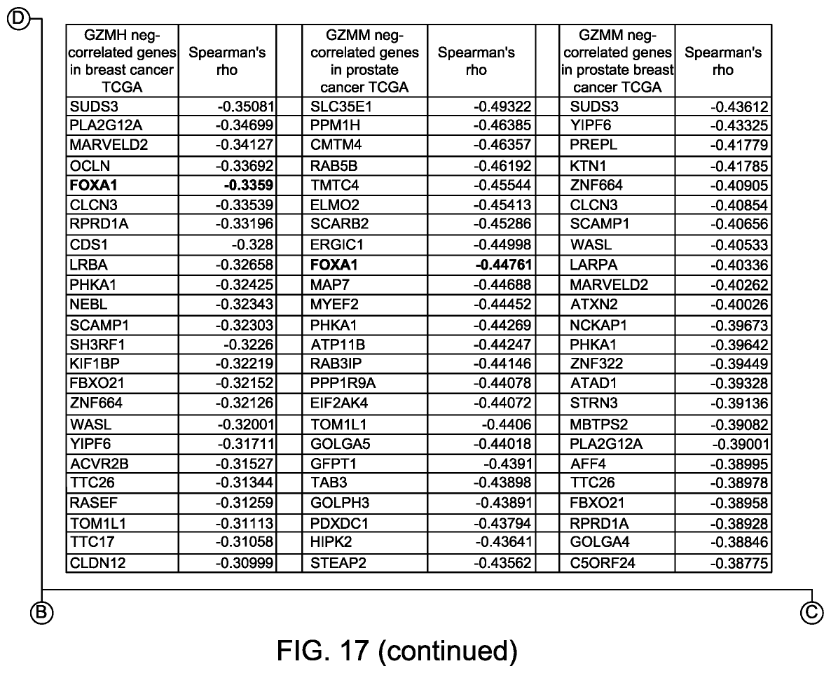

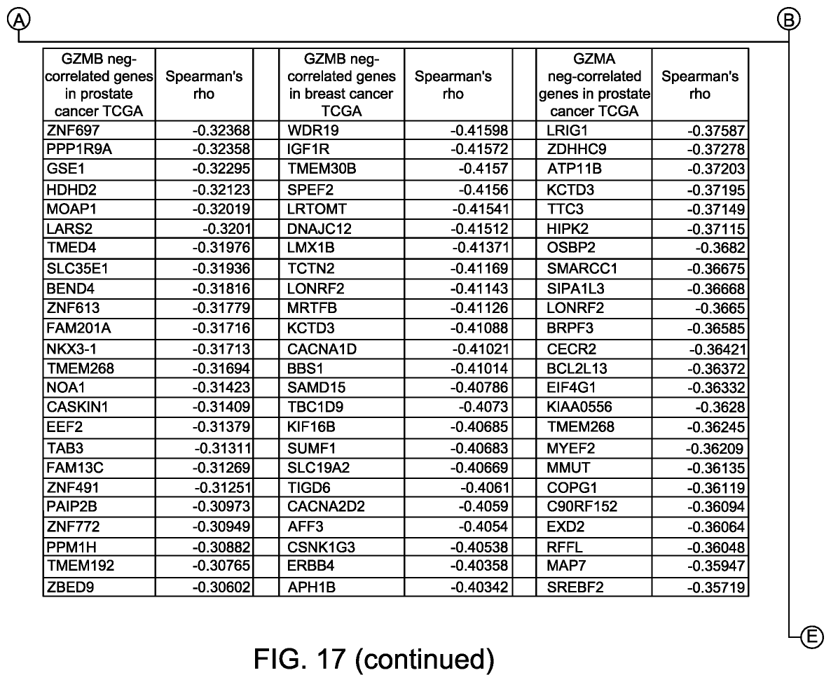

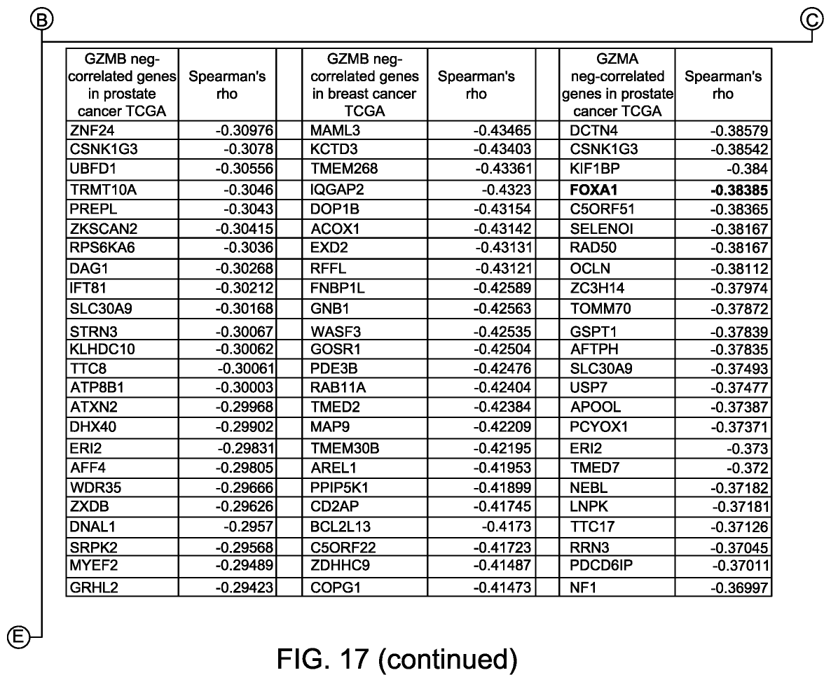

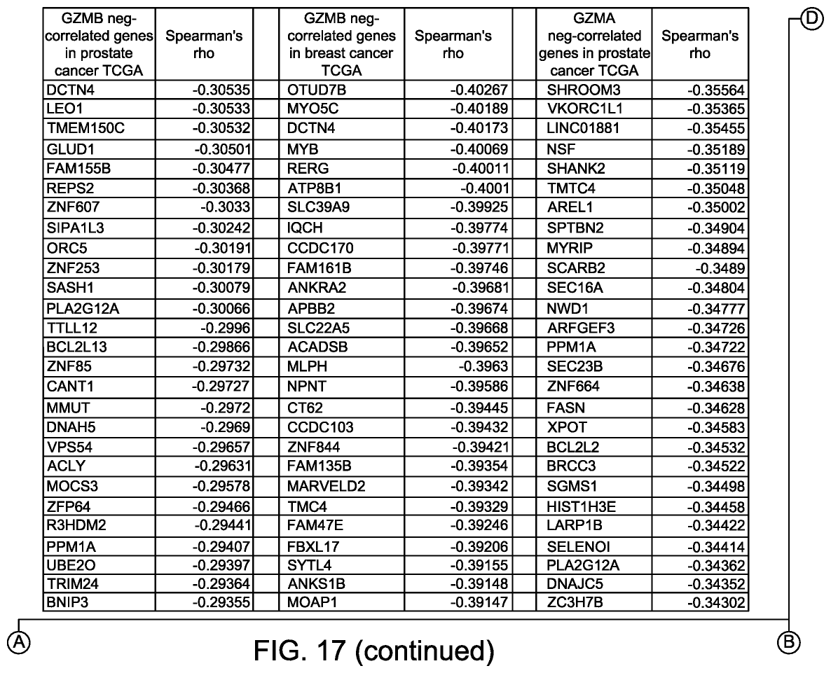

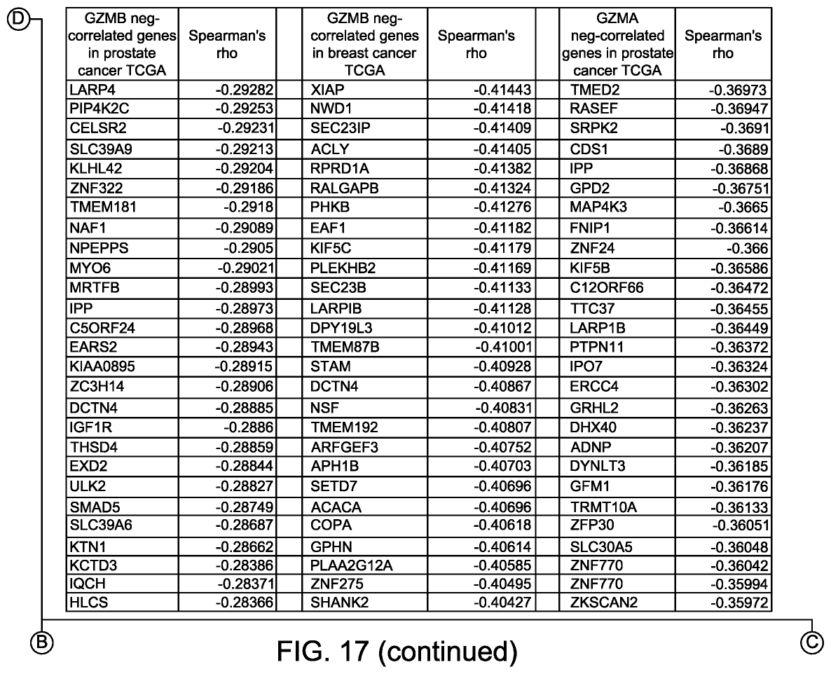

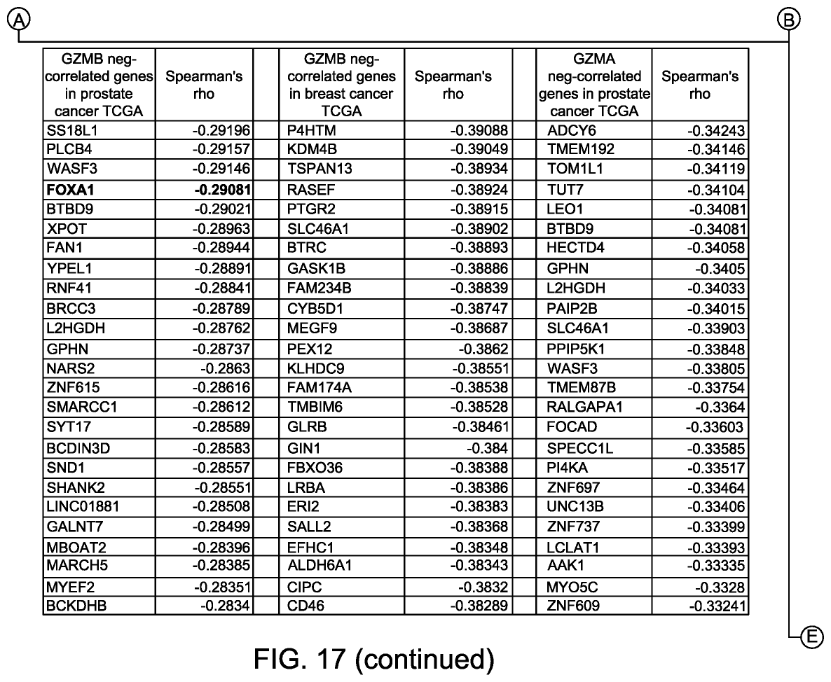

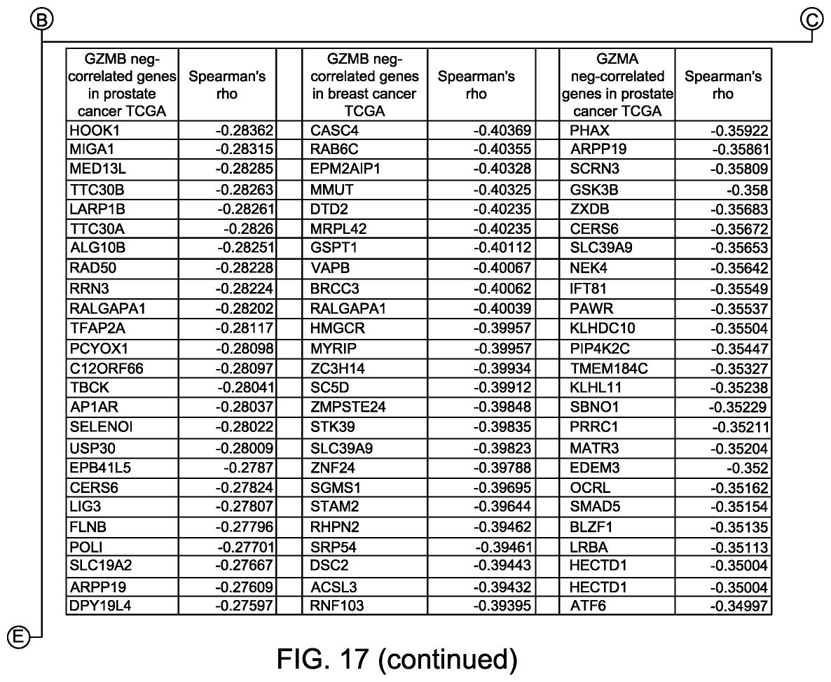

To identify which factors contribute to suppression of infiltration of cytotoxic lymphocytes in immunologically “cold” tumors, RNA-seq expression data from prostate cancer (TCGA Provisional, n =490) and breast cancer (TCGA Provisional, n =960) were used to generate a list of genes whose expression negatively correlated with level of cytotoxic lymphocyte makers (PRF1, GZMs and NKG7) by performing Spearman's rho rank analysis (Table 1 (FIG. 17)). Gene or genes commonly present in the list of top 10 genes in both prostate and breast cancer were considered further as the potential candidate that may be able to suppress cytotoxic lymphocyte infiltration in immunologically “cold” cancers such as prostate and breast cancer. All data was analyzed through cBioPortal (www.cbioportal.org/).

To explore signaling pathways negatively regulated by FOXA1, using the cBioPortal platform (www.cbioportal.org/) and by gene set enrichment analysis online software (software.broadinstitute.org/gsea/msigdb/annotate.jsp), Gene Ontology Biological Process (GO-BP) analysis was performed by examining negative correlation of FOXA1 expression with expression of signaling pathway signature genes in prostate cancer (TCGA Provisional, n=490), metastatic prostate cancer (SU2C/PCF Dream Team, n =270), breast cancer (TCGA Provisional (n=960), and breast cancer from METABRIC database (n=1904).

To investigate the expression correlation between FOXAJ level and expression of CD8⁺ T effector cell (CD8⁺ Teff) signature genes, antigen presentation machinery (APM) genes and type I IFN response signature genes, RNA-seq data of these genes were ranked by increased FOXA1 transcript levels in prostate cancer (n=490, TCGA Provisional), metastatic prostate cancer (n=270, SU2C/PCF Dream Team, source of file: data mRNA seq fpkmpolya.txt), bone metastatic prostate cancer (n=54, dbGaP: phs001141.v1.p1), breast cancer from TCGA Provisional (n=960), breast cancer from METABRIC database (n=1904), and bladder cancer from TCGA (n=404) and heatmaps were generated accordingly. The expression level of CD8⁺ T_{eff} signature genes, APM genes and type I IFN response signature genes were scored as described elsewhere (see, e.g., He et al., Nucleic Acids Res 46:1895-1911 (2018)) and the Pearson's r-values and P-values the correlation with FOXA1 expression were calculated respectively. The CD8⁺ T_{eff} signature genes (BCL11B, CD3D, CD3E, CD8A, CXCR3, GZMA, GZMB, GZMK, IL7R, KLRG1, NKG7, PRF1, TBX21), APM genes (B2M, HLA-A, HLA-B HLA-C, HLA-DPA1, HLA-DPB1, HLA-DQB1, HLA-DRA, HLA-DRB1, HLA-DRB5, HLA-DRB6, HLA-E, HLA-F, HLA-G, HLA-H, HLA-J, PSMB8, PSMB9, TAP1, TAP2, TAPBP) and type I IFN response signature genes (ACACB, BIRC3, BST2, CXCL1, CXCL2, DDX60, DHX58, GBP1, HERC5, IFI16, IFI27, IFI44, IFI44L, IFIH1, IFIT3, IFITM1, IRF7, ISG15, ISG20 ,LGALS9, MX1, OAS1, OAS2, PARP12, RASGRP3 , SAMD9 , SERPING1, SLC15A3, SP110, STAT1, XAF1) were included as much as possible unless the expression data is not available from the dataset. The gene expression data from TCGA were all downloaded from GDC database using R package “TCGAbiolinks,” which is the normalized RSEM expression.

Cell Lines and Cell Culture

LNCaP, VCaP, PC3, DU145, 22RV1, C4-2, C4-2B, LAPC4, BPH1, RWPE-1, TRAMP-C2, MCF7, RT4 and 293T cell lines were purchased from ATCC. LNCaP-RF cell line was derived from LNCaP and cultured in charcoal-stripped medium. LNCaP, VCaP, PC3, DU145, 22RV1, C4-2, C4-2B and LAPC4 cells were maintained in RPMI 1640 containing 10% fetal bovine serum (FBS) and 1% antibiotic/antimycotic (Thermo Fisher Scientific). BPH1, TRAMP-C2, MCF7and 293T cells were maintained in DMEM medium with 10% FBS and 1% antibiotic/antimycotic (Thermo Fisher Scientific). RT4 cells were maintained in McCoy's 5A medium with 10% FBS and 1% antibiotic/antimycotic (Thermo Fisher Scientific). RWPE-1 cells were maintained in keratinocyte serum-free medium (# 17005042, Thermo Fisher Scientific) and 1% antibiotic/antimycotic (Thermo Fisher Scientific). All cells were incubated in an environment of 5% CO₂ at 37° C.

### Luciferase Reporter Assay for Interferon (IFN)-Stimulated Response Activity

To analyze the interferon-stimulated response activity, 293T cells were transfected, using lipofectamine 2000 (Invitrogen) according to the manufacturer's instructions, with the following plasmids: interferon-stimulated response element luciferase reporter (ISRE-luc) containing type I and III IFN response elements, IFN-γ-activated sequence luciferase reporter (GAS-luc) containing type II IFN response elements, Renilla-luc (phRL-TK) as internal control reporter, FOXA1-WT or FOXA1 mutants. At 24 hours after transfection, the transfected cells were treated with 50 ng/L or 100 ng/L IFNα (Sigma-Aldrich, # 14276) or IFNγ (Sigma-Aldrich, # SRP3058) for 5 hours. Renilla and firefly activities were measured with luminometry using the Dual-Luciferase Reporter Assay System (Promega) and the ratio was calculated. Results were expressed as the ratio of firefly to Renilla luciferase activity.

### Real-Time RT-qPCR

LNCaP, VCaP, PC3, DU145, 22RV1, LNCaP-RF, C4-2, C4-2B, LAPC4, BPH1, RWPE-1 and 293T cell lines were treated with or without 10 μg/L IFNα (SigmaAldrich, # 14276) for 24 hours and RNA was isolated using Trizol Reagent (Thermo Fisher Scientific, # 15596018). RNA was eluted in RNase-Free H₂O and reverse-transcribed to cDNA following the kit protocol (Thermo Fisher Scientific, # FERK1672). Gene expression was determined by real-time quantitative PCR (qPCR) using Power SYBR Green (Thermo Fisher Scientific, # 4368708). Primer sequences used for RT-qPCR were as listed in Table 2.

TABLE 2
Primers for q-PCR.

| | | | | Product |
|---|---|---|---|---|
| Names | Sequence (5′-3′) | SEQ ID NO: | Used for | size (bp) |
| GAPDH-F | CCGGGAAACTGTGGCGTGATGG | 4 | RT-qPCR | 309 |
| GAPDH-R | AGGTGGAGGAGTGGGTGTCGCTGTT | 5 | RT-qPCR | |
| FOXA1-F | AGATGGAAGGGCATGAAACCA | 6 | RT-qPCR | 160 |
| FOXA1-R | ATGTTGCCGCTCGTAGTCAT | 7 | RT-qPCR | |
| IFI27-F | GTTCATCCTGGGCTCCATTG | 8 | RT-qPCR | 101 |
| IFI27-R | CCCCTGGCATGGTTCTCTT | 9 | RT-qPCR | |
| HLA-ABC-F | GAGAACGGGAAGGAGACGC | 10 | RT-PCR | 305 |
| HLA-ABC-R | CATCTCAGGGTGAGGGGCT | 11 | RT-PCR | |
| PSMB9-F | GAGAGGACTTGTCTGCACATC | 12 | RT-PCR | 148 |
| PSMB9-R | GCATCCACATAACCATAGATAAAGG | 13 | RT-PCR | |
| TAP1-F | AGAAGGTGGGAAAATGGTACC | 14 | RT-PCR | 119 |
| TAP1-R | GTTGGCAAAGCTTCGAACTG | 15 | RT-PCR | |
| STAT1-F | CAGCTTGACTCAAAATTCCTGGA | 16 | RT-qPCR | 248 |
| STAT1-R | TGAAGATTACGCTTGCTTTTCCT | 17 | RT-qPCR | |
| IFITM1-F | CACGCAGAAAACCACACTTC | 18 | RT-qPCR | 77 |
| IFITM1-R | TGTTCCTCCTTGTGCATCTTC | 19 | RT-qPCR | |
| IFI44-F | TGGTACATGTGGCTTTGCTC | 20 | RT-qPCR | 150 |
| IFI44-R | CCACCGAGATGTCAGAAAGAG | 21 | RT-qPCR | |
| IFI44L-F | TGCACTGAGGCAGATGCTGCG | 22 | RT-qPCR | 156 |
| IFI44L-R | TCATTGCGGCACACCAGTACAG | 23 | RT-qPCR | |
| IFI16-F | ACTGAGTACAACAAAGCCATTTGA | 24 | RT-qPCR | 432 |
| IFI16-R | TTGTGACATTGTCCTGTCCCCAC | 25 | RT-qPCR | |
| PSMB8-F | CCTACATTAGTGCCTTACGGG | 26 | RT-PCR | 143 |
| PSMB8-R | TCCATTTCGCAGATAGTACAGC | 27 | RT-PCR | |
| IRF7-F | GAGCCGTACCTGTCACCCT | 28 | RT-PCR | 146 |
| IRF7-R | GGGCCGTATAGGAACGTGC | 29 | RT-PCR | |
| ISG15-F | GCGCAGATCACCCAGAAGAT | 30 | RT-PCR | 175 |
| ISG15-R | TCCTCACCAGGATGCTCAGA | 31 | RT-PCR | |
| MX1-F | AGACAGGACCATCGGAATCT | 32 | RT-PCR | 109 |
| MX1-R | GTAACCCTTCTTCAGGTGGAAC | 33 | RT-PCR | |

### Plasmid Construction and Mutagenesis

Wild-type V5-tagged FOXA1 lentiviral plasmid was purchased from Addgene (# 70090) and cloned into the SFB-tagged pcDNA3.1 or Flag-tagged pcDNA3.1 or pTSIN lentiviral vector using the Phusion High-Fidelity DNA Polymerase (New England Biolabs, # M0530L). FOXA1 hotspot mutations (H247Q, R261G and F266L) and FOXA1 truncation mutations were engineered from the wild-type FOXA1 vector using the KOD-Plus-Mutagenesis Kit (TOYOBO, # KOD-201) according to the manufacturer's instructions. For the FOXA1 luciferase reporter (FOXA1-luc) construct, the DNA fragment 5′-tcgaTGTTTACTTAcagtaTGTTTACTTTatccgTGTTTACATAgtctaTATTTACTTAccata TGTTTGCTTAgtcaTGTTTACTCA-3′ (SEQ ID NO:34) was inserted into pGL4.28 luc2CP/minP/hygro (Pomega). All plasmids were confirmed using Sanger sequencing. Mutant plasmids were further transfected in 293T cells to confirm expression of the mutant proteins.

### Cell Transfection and RNA Interference

293T cells were co-transfected with pTSIN-Vector or pTSIN-FOXA1 WT or mutants lentiviral plasmids along with packing and envelop plasmids by Lipofectamine 2000 according to the manufacturer's instructions. At two days post-transfection, virus particles containing shRNAs were used to infect cells according to the protocol provided by Sigma-Aldrich. The indicated cells were transduced by culturing with a 1:1 mixture of fresh medium and virus supernatant with Polybrene (4 μg/ml final concentration) for 24 hours. For knockdown of FOXA1, cells were transfected using Lipofectamine 2000 with 50 nM FOXA1 siRNA 5′-GAGAGAAAAAAUCAACAGC-3′ (SEQ ID NO:1; siFOXA1# 1, at 3′ UTR region) or 5′-GCACUGCAAUACUCGCCUU-3′ (SEQ ID NO:2; siFOXA1#2, at CDS region)) or non-targeting control siRNA (siCon) 5′-UAGCGACUAAACACAUCAA-3′ (SEQ ID NO:3). Knockdown or transfection efficiency was determined using Western blotting analysis.

### Western Blotting

Cells were lysed and boiled for 10 minutes in sample buffer (2% SDS, 10% glycerol, 10% β-mercaptoethanol, bromophenol blue and Tris-HCl, pH 6.8). Equal amounts of protein (50-100 μg) from cell lysate were denatured in sample buffer (Thermo Fisher Scientific), subjected to SDS-polyacrylamide gel electrophoresis, and transferred to nitrocellulose membranes (Bio-Rad). The membranes were immunoblotted with specific primary antibodies, horseradish peroxidase-conjugated secondary antibodies, and visualized by SuperSignal West Pico Stable Peroxide Solution (# 34577, Thermo Fisher Scientific). The primary antibodies are AR (dilution 1:1000; #sc-816, Santa Cruz Biotechnology), FOXA1 (dilution 1:2000; # ab23738, Abcam), FOXA1 (dilution 1:1000, # sc-101058, Santa Cruz Biotechnology), STAT1 (dilution 1:1000; # 14994S, Cell Signaling Technology), STAT2 (dilution 1:1000; # 72604S, Cell Signaling Technology), Phospho-STAT1 (Tyr701) (dilution 1:1000; # 9167S, Cell Signaling Technology), Phospho-STAT2 (Tyr690) (dilution 1:1000; # 88410S, Cell Signaling Technology), IRF9 (dilution 1:1000; # 76684S, Cell Signaling Technology), ISG15 (dilution 1:500; # sc-166755, Santa Cruz Biotechnology), PARP1 (dilution 1:1000; # 9532S, Santa Cruz Biotechnology), HSP70 (dilution 1:1000; #4873S, Santa Cruz Biotechnology), MHC class I (MHC-I) (dilution 1:500; #sc-55582, Santa Cruz Biotechnology), Flag (dilution 1:1000; # F1804, Sigma-Aldrich) and V5 (dilution 1:1000; # A190-120A, Bethyl Laboratories), HA (dilution 1:1000; # MMS-101R, Covance), and ERK2 (dilution 1:2000; # sc-1647, Santa Cruz Biotechnology).

### Co-Immunoprecipitation (Co-IP), Protein Purification and GST Pulldown Assay

For extraction of nuclear and cytoplasmic proteins from cells, NE-PER Nuclear and Cytoplasmic Extraction Kit (# 78835, Thermo Fisher Scientific) was used according to the manufacturer's instructions. IP buffer (50 mM Tris-HCl pH7.5, 150 mM NaCl, 1% NP40, 0.5% Sodium Deoxycholate) was used to extract whole cell lysate. P rotein A/G agarose (# 20421, Thermo Fisher Scientific) was used for immunoprecipitation of FOXA1 (# ab23738, Abcam) and Flag-tag (dilution 1:1000; # F1804, Sigma-Aldrich). Monoclonal anti-HA agarose (# A2095, Sigma-Aldrich) was used for HA-tag Co-IP. For glutathione-S-transferase (GST) pulldown assay, GST-tagged STAT2 fragment expressed at E. coli was purified by Glutathione Sepharose 4B beads (# 84-239, Genesee Scientific) and incubated with lysate from 293T cells expressing interested proteins, and GST pulldown assays were performed. For protein co-IP, samples eluted in IP butter were incubated with agarose beads and antibodies overnight at 4° C. and washed with 6 times with IP buffer in the following day. Samples were boiled for 10 minutes in 50 μl sample buffer (2% SDS, 10% glycerol, 10% β-mercaptoethanol, bromophenol blue and Tris-HCl, pH 6.8) and subjected to Western blotting.

### Chromatin Immunoprecipitation Sequencing (ChIP-seq) and Bioinformatics Analyses

ChIP experiments were performed as described elsewhere (see, e.g., He et al., Nucleic acids research 46:1895-1911 (2018)). In brief, chromatin was cross-linked for 15 minutes at room temperature with 11% formaldehyde/PBS solution added to cell culture medium. Cross-linked chromatin was sonicated, diluted and immunoprecipitated with Protein A/G agarose (# 20421, Thermo Fisher Scientific) prebound with antibodies at 4° C. overnight. Antibodies for ChIP were STAT2 (2 μg/sample; # 72604S, Cell Signaling Technology), FOXA1 (2 μg/sample; # ab23738, Abcam). Precipitated protein-DNA complexes were eluted and cross-linking was reversed at 65° C. for 12 hours. ChIP-seq libraries were prepared. High-throughput sequencing (51 nt, pair-end) was performed using the Illumina HiSeg™ 4000 platforms. All short reads were mapped to the human reference genome (GRCh38/hg38) using bowtie2 (version 2.1.0) with default configurations. Reads mapped to multiple positions greater than 2 were discarded, and the remained reads were used for peak calling using MACS2 (version 2.0.10) with a P value cutoff of 1e-5 (macs2 call peak -bdg -SPTMR -f BAM -p 1e-5). Peaks located in the blacklists such as centromere regions were removed (sites.google.com/site/anshulkundaje/projects/blacklists). ChIP-seq tag intensity tracks (bedGraph files) were generated by MACS2, and converted into bigWig files using UCSC “wigToBigWig” tool. H eat maps were drawn by deepTools 2.0.

### FOXA1 Luciferase Reporter Assay and Electrophoretic Mobility Shift Assay (EMSA)

For FOXA1 transcriptional activity analysis, 293T cells were transfected with the SFB-tagged pcDNA3.1 vector (control) or different mutants of FOXA1, KLK3 enhancer luciferase reporter and Renilla-luc (phRL-TK, purchased from Promega, as internal control reporter). At 48 hours after transfection, the renilla and firefly luciferase activities were measured with luminometry using the Dual-Luciferase Reporter Assay System (Promega) and the ratio was calculated. Results were expressed as the ratio of firefly to Renilla luciferase activity. For EMSA, 60 base pairs of forkhead response element in the KLK3 enhancer (centered at the FOXA1 consensus binding motif 5′-GTAAACAA-3′: 5′-ACATATTGTATCGATTGTCCTTGACAGTAAACAAATCTGTTGTAAGAGACATT ATCTTTA-3′; SEQ ID NO:35) and ISRE probe (5′-CTCCCCTGAGTTTCACTTCTTCTCCCAACTTG-3′; SEQ ID NO:36) were synthesized from Integrated Device Technology (IDT) and labelled with biotin using Biotin 3′-End DNA labelling kit (# 89818, Thermo Fisher Scientific) and annealed to generate a labelled double stranded DNA duplex. Binding reactions were carried out in 20 μl volumes containing 2 μl of the nuclear lysates, 50 ng/μl poly(dI.dC), 1.25% glycerol, 0.025% Nonidet P-40 and 5 mM MgCl₂. Biotin labelled KLK3 enhancer probe (10 fmol) was added and incubated for 1 hour at room temperature, size-separated on a 6% DNA retardation gel at 100 V for 1 hour in 0.5×TBE buffer, and transferred on the Biodyne Nylon membrane (# 77015, Thermo Fisher Scientific) and crosslinked to the membrane using the UV light at 120 mJ/cm² for 2 minutes. Biotin-labelled free and protein-bound DNA was detected using horseradish peroxidase-conjugated and developed using Chemiluminescent Nucleic Acid Detection Module Kit (# 89880, Thermo Fisher Scientific) according to the manufacturer's protocol.

### Tumor Cell Injections and Treatment

TRAMP-C2-Vector or TRAMP-C2-FOXA1ΔαH3 cells (3×10⁶) were injected subcutaneously into the right flank of 8-week old male C57BL/6 on day 0. Tumors were measured twice per week with calipers and the volume calculated (length×width×width×0.5). Poly I:C (2.5 mg/kg, 100 pl) purchased from Sigma-Aldrich (# P1530) or vehicle (PBS, 100 pl) was administered by intratumoral injection twice per week (five doses in total) and tumors were measured twice per week until the tumor volume reached the maximum allowed size (1,000 cm³). For CyTOF and Immunofluorescence experiments, mice were euthanized at 48 hours post the last administration.

### Single-Cell Mass Cytometry (CyTOF) Analysis

Single tumor cells were isolated using the Mouse Tumor Dissociation Kit (Miltenyi Biotec, # 130-096-730) following standard protocol. CyTOF staining panels are detailed in Table 3.

TABLE 3
Antibodies used for CyTOF.

| Antibodies | Clone | Tag | Source | Localization |
|---|---|---|---|---|
| Anti-Mouse CD45 | 30-F11 | 89Y | Fluidigm, Cat#: 3089005B | surface |
| Anti-Mouse CD3e | 145-2C11 | 152Sm | Fluidigm, Cat#: 3152004B | surface |
| Anti-Mouse CD8a | 53-6.7 | 153-Eu | Fluidigm, Cat#: 3153012B | surface |
| Anti-Mouse CD4 | RM4-5 | 145-Nd | Fluidigm, Cat#: 3145002B | surface |
| Anti-Mouse CD11b | M1/70 | 172-Yb | Fluidigm Cat#: 3172012B | surface |
| Anti-Mouse NK1.1 | PK136 | 170Er | Fluidigm, Cat#: 3170002B | surface |
| Anti-Mouse Ly-6G | 1A8 | 141Pr | Fluidigm, Cat#: 3141008B | surface |
| Anti-Mouse Ly-6C | HK1.4 | 162Dy | Fluidigm, Cat#: 3162014B | surface |
| Anti-Mouse Foxp3 | FJK-16s | 158Gd | Fluidigm, Cat#: 3158003A | intracellular |
| Anti-Mouse Granzyme B | GB11 | 173Yb | Fluidigm, Cat#: 3173006B | intracellular |

Digested tumors were mashed through 40 μm filters into RPMI-1640 and were centrifuged at 300 g for 5 minutes at 4° C. All single cells were depleted of erythrocytes by hypotonic lysis for 1 minute at room temperature. Cells were washed once with PBS and incubated with 0.5 mM cisplatin by diluting 5 mM Cell-IDTM Cisplatin (Fluidigm, # 201064) at for 5 minutes. 5 x 10⁶ or fewer cells per tumor were blocked with FcR Blocking Reagent (Miltenyl Biotec, # 130-059-901) for 10 minutes and incubated with surface antibody mix for 45 minutes at room temperature. Cells were washed with MaxPar Cell Staining Buffer (Fluidigm, # 201068). For intracellular staining, cells were incubated with FOXP3 Fixation/Permeabilization 1× working solution by diluting 4×Fixation/Permeabilization Concentrate (eBioscience, # 00-5123-43) with Fixation/Permeabilization Diluent (eBioscience, # 00-5223-56) at 1:4 dilution for 45 minutes at room temperature (keep in dark). Cells were washed twice with 1× working solution of Permeabilization Buffer (eBioscience, # 00-8333-56). Centrifuge at 800×g for 5 minutes and supernatant was carefully aspirated and re-suspend in 500 μL CyPBS diluted from 10×PBS (Rockland Immunochemicals, # MB-008) in Maxpar water (Fluidigm, # 201069). Samples were fixed with 500 μl 2×fixation solution 4% Paraformaldehyde diluted from 16% Paraformaldehyde Aqueous Solution (Electron Microscopy Sciences, # 915710S) in CyPBS and incubated at 4° C. overnight. Cells were washed with 1 mL Maxpar Cell Staining Buffer (Fluidigm; # 201068) and spin down at 800×g for 5 minutes at room temperature and re-suspended in 1 mL 12.5 nM intercalation solution by diluting 125 μM intercalator stock (Cell-IDTM lntercalator-Ir- 125 μM, Fluidigm, Part No. 201192A) 1:10,000 in Maxpar Fix and Perm Buffer-100 mL (Fluidigm; # 201067). Samples were washed with 1 mL CyPBS and the EQ beads (Fluidigm; # 201078) were added and cells were counted on Countess II and re-suspended to approximately 5×10⁵ cells/mL. Samples were filtered through 35 μm blue cap FACS tube (Falcon, # 352235) and were analyzed with a CyTOF instrument (Fluidigm). Data were analyzed with PhenoGraph by following the instruction. The 1.0.153 version of R studio was downloaded from the official R website (r-project.org/). Data were analyzed with PhenoGraph by following the program instructions. R studio (Version 1.0.136) was downloaded from the official R website (www.r-project.org/). The cytofkit package (Release 3.6) was downloaded from Bioconductor (https://www.bioconductor.org/packages/release/bioc/html/cytofkit.html) and opened in the R studio. Manually gated singlet (19Ir+193Ir+), viable (195Pt +) events were imported into cytofkit, subjected to PhenoGraph analysis, and clustered on the basis of markers, with the following settings: merge each file, transformation: cytofAsinh, cluster method: Rphenograph, visualization method: tSNE (t-distributed stochastic neighbor embedding), and cellular progression: NULL. PhenoGraph identified unique clusters were visualized via the R package “Shiny,” where labels, dot size, and cluster color were customized. Clusters were colored according to phenotype based on the median expression of various markers. The frequency of each cluster was determined via csv files generated by the algorithm. Percentages of each cell populations were analyzed with FlowJo and GraphPad Prism 7 software.

### Immunofluorescence

Formalin-fixed paraffin-embedded TRAMP-C2 tumor samples were deparaffinized, rehydrated and subjected to heat-mediated antigen retrieval. Sections were incubated with 1% Sudan Black (dissolved in 70% ethanol) for 20 minutes at room temperature to reduce autofluorescence. Slides were washed with 0.02% Tween 20, incubated with 0.1 M Glycine for 10 minutes, and immersed slides in 10 mg/mL Sodium Borohydride in ice cold Hanks Buffer on ice for 40 minutes. After washing with two times PBS, slides were blocked by 1% BSA in PBS for 30 minutes and incubated with FOXA1 antibody (1:1000 dilution; Abcam, # ab170933) at 4° C. overnight. The sections were washed three times in 1X PBS and treated for 30 minutes with goat anti-Rabbit IgG (H+L) Highly Cross-Adsorbed Secondary Antibody-Alexa Fluor 594 (1:500 dilution; Invitrogen, # A-11037). Prior to imaging, samples were mounted with VECTASHIELD Antifade Mounting Medium with DAPI (Fisher Scientific, # NC9524612). Samples were imaged using Nikon spinning disk confocal.

Analysis of FOXA1 mRNA Expression and Immune Cell Markers in Murine TNBC Samples Treated with Immunotherapy and Breast Cancer of Patients Treated with Chemotherapy

To evaluate the mRNA expression level of Foxal and immune cell markers (CD3e, CD8a and Gzmb) in immunotherapy resistant and sensitive samples, RNA-seq data (GSE124821) from triple-negative breast cancer murine models treated with anti-PD1 and anti-CTLA-4 combination therapy was analyzed. To evaluate the mRNA expression level of FOXA1, CD3E, CD8A and GZMB in patients with pathological complete response (pCR with residual cancer burden (RCB) 0 or I) and no pathological complete response (No pCR with RCB II or III) to neoadjuvant chemotherapy (NAC) in breast cancer, RNA-seq data from Breast Cancer Genome-Guided Therapy (BEAUTY) (Goetz et al., J Natl Cancer Inst 109(7): djw306 (2017)) project was analyzed. To further validate the data from this cohort, RNA-microarray data from NAC-treated breast cancer (NCT00455533; GSE41998) from an independent cohort (Horak et al., Clin Cancer Res 19:1587-1595 (2013)) was also analyzed.

Clinical Data and Patient Information of Urothelial Carcinomas Treated with Immunotherapy

Tumor samples and medical records from a cohort of 23 patients (20 males and 3 females; Age from 44 to 77 years; Median age 65 years) with urothelial carcinoma (cancers in bladder, renal pelvis, ureter or urethra that showed predominantly transitional-cell features on histologic testing) were analyzed. Urothelial carcinoma samples were obtained from the primary or metastatic lesions of 23 patients before they underwent therapy with anti-PD1 treatment until disease progression according to the Response Evaluation Criteria in Solid Tumors (RECIST) version 1.1,15 (Eisenhauer et al., Eur J Cancer 45:228-247 (2009)). Formalin-fixed paraffin-embedded (FFPE) tumor specimens with sufficient viable tumor content were required before the start of the study. One sample (patient 7) was excluded for immunohistochemistry (IHC) staining evaluation because the specimen was too small. The total specimens for the FOXA1 IHC staining evaluation are 22 (see Table 4 (FIG. 18) and FIG. 15).

### Immunohistochemistry (IHC) of Urothelial Carcinoma Patient Specimens

Urothelial carcinoma FFPE samples were deparaffinized, rehydrated and subjected to heat-mediated antigen retrieval. Sections were incubated with 3% H₂O₂ for 15 minutes at room temperature to quench endogenous peroxidase activity. After antigen retrieval using unmasking solution (Vector Labs), slides were blocked with normal goat serum for 1 hour and incubated with primary antibody at 4° C. overnight. IHC analysis of tumor samples was performed using primary antibodies for FOXA1 (dilution 1:500; Abcam, # ab170933). The sections were washed three times in 1X PBS and treated for 30 minutes with biotinylated goat-anti-rabbit IgG secondary antibodies (#BA-9200, Vector Labs). After washing three times in 1× PBS, sections were incubated with streptavidin-conjugated HRP (# 3999S, Cell Signaling Technology). After washing three times in 1X PBS for 5 minutes each, specific detection was developed with 3,3′3diaminobenzidine (# D4168-50SET, Sigma-Aldrich). For IHC staining score (IS) or intensity, 0=<1% positive cells, 1=1-20% positive cells, 2=20-50% positive cells, 3 =>50% positive cells. FOXA1 expression levels with IS=0 or 1 are considered as “low” and IS =2 or 3 are considered as “high” (see Table 4 (FIG. 18) and FIG. 15).

Statistical Analysis

GraphPad Prism 7 was used for statistical analyses of results from RT-qPCR, luciferase reporter and cell proliferation assays. P values from unpaired two-tailed Student's t tests were used for comparisons between two groups and one-way ANOVA with Bonferroni's post hoc test was used for multiple comparisons. Statistical analysis is specifically described in figure legends. P value <0.05 was considered significant.

### Data Availability

The GEO accession number for the ChIP-seq data is GSE142221: www.ncbi.nlm.nih.gov/geo/query/acc.cgi?acc=GSE142221; Code: yderayksdtazlsr.

### Example 2

### FOXA1 ASOs Sensitize Cancer to Immunotherapy

### Results

### FOXA1 ASOs Effectively Downregulate Foxa1 Protein in Murine Prostate Cancer Cells

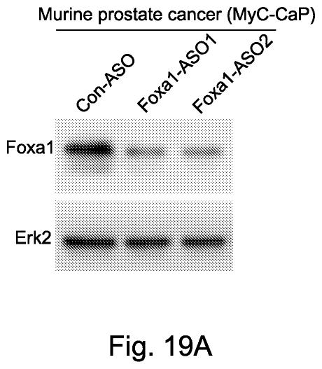

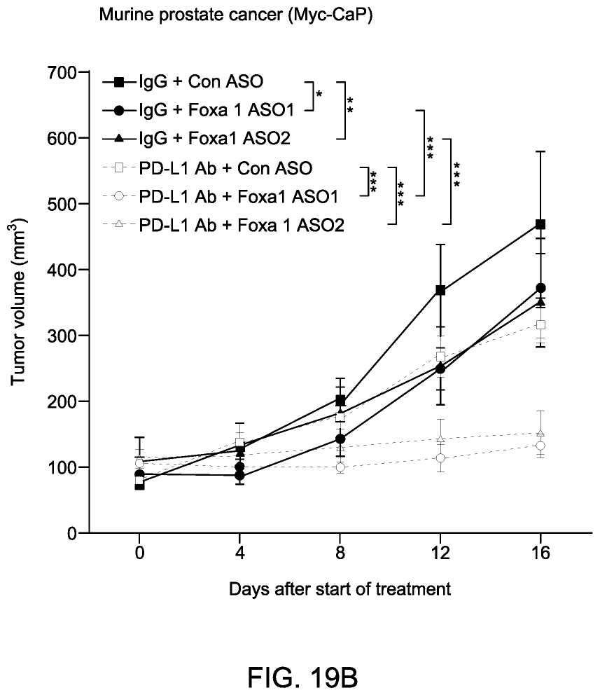

MyC-CaP murine prostate cancer cells were transfected with control ASO (Con ASO) or two Foxal-specific ASOs (Foxal ASO1 and Foxal ASO2). As shown in FIG. 19A, both Foxal ASOs substantially knocked down Foxal protein in these cells, indicating that Foxal ASOs can effectively downregulate Foxal proteins in MyC-CaP cells.

### FOXA1 ASOs Enhances Anti-Cancer Effect of Anti-PD-L1 Antibody in Mice

MyC-CaP cells (3×10⁶) were injected subcutaneously into the right flank of 6-week-old wild-type intact FVB male mice. When the average tumor volume reached approximately 100 mm³, mice were randomized into groups subsequently treated with intraperitoneal injection of anti-PD-L1 or non-specific control IgG (10 mg/kg) in combination with control antisense oligonucleotides (12.5 mg/kg), Foxal ASO1 (12.5 mg/kg), or Foxal-ASO2 (12.5 mg/kg). As shown in FIG. 19B, treatment of mice with Foxa1 ASOs significantly inhibited tumor growth in mice. Administration of anti-PD-L1 antibody also inhibited MyC-CaP tumor growth (FIG. 19B). Co-treatment of mice with Foxa1 ASO with PD-L1 antibody significantly suppressed tumor growth compared to mice treated with each agent alone (FIG. 19B). These data indicate that depletion of FOXA1 by antisense oligonucleotides largely enhances immunotherapy efficacy of anti-PD-L1 in prostate cancer

### Materials and Methods

Antisense oligonucleotides (ASOs)
Foxa1 ASO1:
(SEQ ID NO: 42)
5′-+G*+G*+T*A*G*C*G*C*C*A*T*A*A*G*G*A*G*+A*+G*+T-
3′
Foxa1 ASO2:
(SEQ ID NO: 43)
5′-+T*+G*+G*A*T*G*G*C*C*A*T*C*G*+T*+G*+A-3′
Control ASO:
(SEQ ID NO: 44)
5′-+G*+A*+C*G*C*G*C*C*T*G*A*G*A*G*+G*+T*+T-3′
+indicates that the nucleotide immediately
following the “+” symbol is a locked nucleic acid
(LNA) in which the ribose moiety is modified with
an extra methylene bridge connecting the 2′ oxygen
and 4′ carbon.
*indicates that the nucleotide immediately prior
to the “*” symbol has a phosphorothioate (PS)
backbone. The ASOs were custom synthesized.

### Cell Lines

The MyC-CaP murine prostate cancer cell line, originally derived from prostate tumors of Hi-Myc transgenic mice in FVB genetic background, was purchased from ATCC (Manassas, Va.).

### Antibodies

Antibodies used include anti-FOXA1 antibody (# ab23738, Abcam), anti-ERK2 (# sc-1647, Santa Cruz Biotechnology), anti-mouse PD-L1 mAb (clone 10B5), and InVivoMAb mouse IgG1 isotype control (clone MOPC-21) (# BE0083, Bio X Cell).

### Animal Sstudies

Thirty six 6-week-old wild-type intact FVB mice were purchased from Jackson Laboratories (Bar Harbor, Me.). Tumor volume was measured by digital caliper and calculated using a formula of length×width×width×0.5.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-10. (canceled)

11. A method for treating a mammal having cancer, wherein said method comprises:
(a) detecting an increased level of FOXA1 polypeptide expression in a sample obtained from said mammal; and
(b) administering a cancer treatment to said mammal, wherein said cancer treatment is not an immunotherapy or a chemotherapy.

12. (canceled)

13. The method of claim 11, wherein said mammal is a human.

14. The method of claim 11, wherein said sample comprises cancer cells of said cancer.

15. The method of claim 11, wherein said cancer is selected from the group consisting of a prostate cancer, a breast cancer, a bladder cancer, a lung cancer, a liver cancer, a cervical cancer, a bile duct cancer, a colon cancer, a rectal cancer, a pancreatic cancer, a uterine cancer, a head and neck cancer, a testicular cancer, a ovarian cancer, a thyroid cancer, a bone cancer, a skin cancer, an adrenal gland cancer, a kidney cancer, a lymphoma, a thymus cancer, a brain cancer, a leukemia, and a cancer of the eye.

16. The method of claim 11, wherein said cancer treatment comprises surgery or radiation treatment.

17. (canceled)

18. A method for treating a mammal having cancer, wherein said method comprises:
(a) detecting an absence of an increased level of FOXA1 polypeptide expression in a sample obtained from said mammal; and
(b) administering a cancer treatment to said mammal, wherein said cancer treatment is an immunotherapy or a chemotherapy.

19. (canceled)

20. The method of claim 18, wherein said mammal is a human.

21. The method of claim 18, wherein said sample comprises cancer cells of said cancer.

22. The method of claim 18, wherein said cancer is selected from the group consisting of a prostate cancer, a breast cancer, a bladder cancer, a lung cancer, a liver cancer, a cervical cancer, a bile duct cancer, a colon cancer, a rectal cancer, a pancreatic cancer, a uterine cancer, a head and neck cancer, a testicular cancer, a ovarian cancer, a thyroid cancer, a bone cancer, a skin cancer, an adrenal gland cancer, a kidney cancer, a lymphoma, a thymus cancer, a brain cancer, a leukemia, and a cancer of the eye.

23. The method of claim 18, wherein said cancer treatment comprises an immunotherapy selected from the group consisting of pembrolizumab, nivolumab, cemiplimab, spartalizumab, camrelizumab, sintilimab, tislelizumab, toripalimab, AMP-224, AMP-514, atezolizumab, avelumab, durvalumab, KN035, CK-301, AUNP12, CA-170, and BMS-986189.

24. The method of claim 18, wherein said cancer treatment comprises a chemotherapy selected from the group consisting of actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, and vindesine.

25. A method for treating a mammal having cancer, wherein said method comprises:
(a) detecting an increased level of FOXA1 polypeptide expression in a sample obtained from the mammal;
(b) administering an inhibitor of a FOXA1 polypeptide; and
(c) administering a cancer treatment to said mammal, wherein said cancer treatment is an immunotherapy or a chemotherapy.

26. (canceled)

27. The method of claim 25, wherein said mammal is a human.

28. The method of claim 25, wherein said sample comprises cancer cells of said cancer.

29. The method of claim 25, wherein said cancer is selected from the group consisting of a prostate cancer, a breast cancer, a bladder cancer, a lung cancer, a liver cancer, a cervical cancer, a bile duct cancer, a colon cancer, a rectal cancer, a pancreatic cancer, a uterine cancer, a head and neck cancer, a testicular cancer, a ovarian cancer, a thyroid cancer, a bone cancer, a skin cancer, an adrenal gland cancer, a kidney cancer, a lymphoma, a thymus cancer, a brain cancer, a leukemia, and a cancer of the eye.

30. The method of claim 25, wherein said inhibitor of said FOXA1 polypeptide is an inhibitor of FOXA1 polypeptide activity selected from the group consisting of SNS-032 (BMS-387032), Ro 31-8220, Aurora A Inhibitor I, WZ8040, Dasatinib, Lapatinib, Saracatinib (AZD0530), JNK-IN-8, BI 2536, Crenolanib (CP-868596), Herceptin, CYT387, BEZ235 (Dactolisib), PHA-793887, NVP-BSK805 2HCl, Cediranib (AZD2171), PF-00562271, Flavopiridol, AT7519, Apicidin, and Volasertib (BI 6727).

31. (canceled)

32. The method of claim 25, wherein said inhibitor of said FOXA1 polypeptide is an inhibitor of FOXA1 polypeptide expression selected from a small interfering RNA (siRNA) molecule and an antisense oligo.

33-34. (canceled)

35. The method of claim 32, wherein administering said inhibitor of said FOXA1 polypeptide comprises administering a viral particle comprising said siRNA to said mammal.

36. (canceled)

37. The method of claim 25, wherein said cancer treatment comprises an immunotherapy selected from the group consisting of pembrolizumab, nivolumab, cemiplimab, spartalizumab, camrelizumab, sintilimab, tislelizumab, toripalimab, AMP-224, AMP-514, atezolizumab, avelumab, durvalumab, KN035, CK-301, AUNP12, CA-170, and BMS-986189.

38. The method of claim 25, wherein said cancer treatment comprises a chemotherapy selected from the group consisting of actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, and vindesine.
